(19) United States
(12) Patent Application Publication
(10) Pub. No.: US 2023/0338421 A1
(43) Pub. Date: Oct. 26, 2023

(54) COMPOSITIONS AND METHODS FOR AUTOIMMUNITY REGULATION

(71) Applicant: TCR2 THERAPEUTICS INC., Cambridge, MA (US)

(72) Inventors: Daniel GETTS, Westminster, MA (US); Robert HOFMEISTER, Scituate, MA (US); Patrick Alexander BAEUERLE, Gauting (DE); Dario GUTIERREZ, Waltham, MA (US); Dana GILMORE, Boston, MA (US)

(21) Appl. No.: 17/791,358

(22) PCT Filed: Jan. 8, 2021
(86) PCT No.: PCT/US2021/012748

§ 371 Date: Jul. 7, 2022

Related U.S. Application Data
(60) Provisional application No. 62/959,794, filed on Jan. 10, 2020.
(60) Provisional application No. 63/094,590, filed on Oct. 21, 2020.

(51) Int. Cl. A61K 35/17; C07K 14/725; C07K 14/715; C12N 15/86; A61P 37/02

(52) U.S. Cl. CPC A61K 35/17; C07K 14/7051; C07K 14/715; C12N 15/86; A61P 37/02

(57) ABSTRACT

Provided herein are recombinant nucleic acids encoding T cell receptor (TCR) fusion proteins (TFPs), modified human immune cells expressing the encoded molecules, and methods of use thereof for the treatment of diseases, including autoinmmune diseases.

### CROSS-REFERENCE

This application claims the benefit of U.S. Provisional Patent Application No. 62/959,794, filed Jan. 10, 2020, and U.S. Provisional Patent Application No. 63/094,590, filed Oct. 21, 2020, each of which is entirely incorporated herein by reference.

### BACKGROUND

Regulatory T cells (Tregs) can be central to immune system homeostasis and play a major role in maintaining tolerance to self-antigens and in modulating the immune response to foreign antigens. Multiple autoimmune and inflammatory diseases, including Type 1 Diabetes (T1D), Systemic Lupus Erythematosus (SLE), and Graft-versus-Host Disease (GVHD) have been shown to have a deficiency of Treg cell numbers or Treg function.

The immunosuppressive properties of Tregs can make them attractive candidates for cellular therapy, particularly for application in conditions such as hematopoietic stem cell transplantation (HSCT), solid organ transplantation, and autoimmunity.

### SUMMARY

Recognized herein is a need for autoimmune disease therapies that can potentiate regulatory T cell (Treg) numbers and functions.

In an aspect, the present disclosure provides a pharmaceutical composition comprising (I) a T regulatory cell (Treg) from a human subject, wherein the T regulatory cell comprises: (a) a recombinant nucleic acid molecule encoding a T cell receptor (TCR) fusion protein (TFP) comprising (i) a TCR-integrating subunit comprising (1) an extracellular domain, (2) a TCR transmembrane domain, and (3) a TCR intracellular domain comprising a stimulatory domain from an intracellular signaling domain; (ii) a binding domain; and (II) a pharmaceutically acceptable carrier; wherein the TCR-integrating subunit and the binding domain are operatively linked; and wherein the TFP functionally interacts with an endogenous TCR when expressed in a T cell.

In some embodiments, the binding domain is selected from: an antigen binding domain; a T cell receptor ligand, e.g., a peptide-MHC complex; or a T cell receptor mimic, e.g., that binds the peptide-MHC complex.

In some embodiments, the Treg further comprises a gene that stimulates and/or stabilizes the formation of Tregs. In some embodiments, the gene that stimulates and/or stabilizes the formation of Tregs is encoded by the same recombinant nucleic acid molecule as the recombinant nucleic acid molecule encoding the TFP. In some embodiments, the gene that stimulates and/or stabilizes the formation of Tregs is encoded by a different recombinant nucleic acid molecule than the recombinant nucleic acid molecule encoding the TFP. In some embodiments, the gene that stimulates and/or stabilizes the formation of Tregs is FOXP3, HELIOS, BACH2, or pSTAT5. In some embodiments, the Treg further comprises a switch receptor.

In some embodiments, the switch receptor is encoded by the same recombinant nucleic acid molecule as the recombinant nucleic acid molecule encoding the TFP. In some embodiments, the switch receptor is encoded by a different recombinant nucleic acid molecule than the recombinant nucleic acid molecule encoding the TFP. In some embodiments, the switch receptor is an IL7-IL2 switch receptor, an IL7-IL10 switch receptor, or a TNF-alpha-IL2 switch receptor. In some embodiments, the Treg comprises more than one gene that stimulates and/or stabilizes the formation of Tregs and/or more than one switch receptor. In some embodiments, the expression of one or more of PKC theta, STUB1, and CCAR2 in the Treg cell is reduced or eliminated.

In some embodiments, the expression of one or more of CDK8 and CDK19 reduced, deleted, or pharmacologically inhibited to stabilized Treg formation.

In some embodiments, the peptide of the peptide-MHC complex is an autoantigen or a fragment thereof. In some embodiments, the peptide of the peptide-MHC complex is an exogenous antigen or a fragment thereof. In some embodiments, the binding domain comprises an antigen binding domain. In some embodiments, the antigen binding domain comprises an autoantigen binding domain or an exogenous antigen binding domain. In some embodiments, the autoantigen binding domain specifically binds an autoantigen. In some embodiments, the exogenous antigen binding domain specifically binds an exogenous antigen. In some embodiments, the autoantigen is one or more of islet glucose-6-phosphatase catalytic subunit related protein (IGRP), insulin, HLA-A2, myelin, or alpha-gliadin or a fragment thereof. In some embodiments, the exogenous antigen is FVIII or a therapeutic macromolecule, e.g., a therapeutic polypeptide, or a fragment thereof. In some embodiments, the antigen binding domain binds to a cell membrane associated antigen. In some embodiments, the antigen binding domain binds to a circulating antigen. In some embodiments, the antigen binding domain is specific to an antigen on an islet cell. In some embodiments, the antigen binding domain is an antibody. In some embodiments, the antibody is an scFv or a single domain antibody. In some embodiments, the antibody is human or humanized. In some embodiments, the binding domain is a TCR mimic, e.g., specifically binds a peptide-MHC-complex.

In some embodiments, the pharmaceutical composition reduces cytokine production of an effector T cell having the antigen, the MHC-peptide complex, or the T cell receptor that specifically binds the MHC-peptide complex, relative to a pharmaceutical composition having a Treg that does not contain the TFP.

In some embodiments, the Treg is a CD4⁺ CD25⁺ FoxP3⁺ Treg or a CD8⁺ regulatory T cell.

In some embodiments, the intracellular signaling domain is selected from CD3 gamma, CD3 delta, CD3 epsilon, and CD3 zeta. In some embodiments, the TCR-integrating subunit comprises (i) a TCR extracellular domain, (ii) a TCR transmembrane domain, and (iii) a TCR intracellular domain, wherein at least two of (i), (ii), and (iii) are from the same TCR subunit. In some embodiments, the encoded binding domain is connected to the TCR extracellular domain by a linker sequence. In some embodiments, the encoded linker sequence comprises (G4S)n, wherein n=1 to 4.

In some embodiments, the TFP includes an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some embodiments, the encoded TFP includes a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, a CD3 zeta TCR subunit, functional fragments thereof, and amino acid sequences thereof having at least one but not more than 20 modifications.

In some embodiments, the TFP includes an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the ITAM replaces an ITAM of CD3 gamma, CD3 delta, or CD3 epsilon.

In another aspect, the present disclosure provides a recombinant nucleic acid molecule encoding the TFP described herein. In some embodiments, the nucleic acid is selected from the group consisting of a DNA and an RNA. In some embodiments, the nucleic acid is an mRNA. In some embodiments, the nucleic acid is circular RNA (circRNA).

In some embodiments, the recombinant nucleic acid molecule comprises a nucleic acid analog, wherein the nucleic acid analog is not in an encoding sequence of the recombinant nucleic acid. In some embodiments, the recombinant nucleic acid molecule further comprises a leader sequence. In some embodiments, the recombinant nucleic acid molecule further comprises a promoter sequence. In some embodiments, the recombinant nucleic acid molecule further comprises a sequence encoding a poly(A) tail. In some embodiments, the recombinant nucleic acid molecule further comprises a 3′UTR sequence. In some embodiments, the nucleic acid is an isolated nucleic acid or a non-naturally occurring nucleic acid. In some embodiments, the nucleic acid is an in vitro transcribed nucleic acid.

In another aspect, the present disclosure provides a vector comprising the recombinant nucleic acid molecule described herein. In some embodiments, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some embodiments, the vector is an in vitro transcribed vector.

In another aspect, the present disclosure provides a circular RNA comprising the recombinant nucleic acid molecule described herein.

In another aspect, the present disclosure provides a method treating or preventing a disease or disorder comprising administering to a subject in need thereof a therapeutically effective amount of the pharmaceutical composition described herein. In some embodiments, the disease or disorder is an autoimmune disease. In some embodiments, the autoimmune disease is an autoantibody-mediated autoimmune disease. In some embodiments, the autoimmune disease is selected from the group comprising multiple sclerosis, autoimmune hemolytic anemia, celiac disease, and chronic inflammatory demyelinating polyradiculoneuropathy. In some embodiments, the disease or disorder is inflammation, e.g., an inflammatory disease or disorder, an allergic reaction, or transplant rejection.

In another aspect, the present disclosure provides a composition for use in treating or preventing a disease or disorder in a subject in need thereof, comprising administering to the subject an effective amount of the pharmaceutical composition described herein. In some embodiments, the disease or disorder is an autoimmune disease. In some embodiments, the disease or disorder is inflammation, e.g., an inflammatory disease or disorder, an allergic reaction, or transplant rejection. In some embodiments, the subject has or is at risk of developing an autoimmune disease, inflammation, e.g., an inflammatory disease or disorder, an allergic reaction, or transplant rejection.

In another aspect, the present disclosure provides a T regulatory cell (Treg) from a human subject, wherein the T regulatory cell comprises a recombinant nucleic acid comprising a sequence encoding a T cell receptor (TCR) fusion protein (TFP) comprising:

(i) a TCR-integrating subunit comprising: (1) at least a portion of a TCR extracellular domain, and (2) a TCR transmembrane domain, and (ii) a binding domain; wherein the TCR-integrating subunit and the binding domain are operatively linked; and wherein the TFP functionally interacts with an endogenous TCR when expressed in a T cell.

In some embodiments, the TFP further comprises a TCR intracellular signaling domain. In some embodiments, the binding domain is selected from: an antigen binding domain: a T cell receptor ligand, e.g., a peptide-MHC complex; or a T cell receptor mimic, e.g., that binds the peptide-MHC complex. In some embodiments, the Treg further comprises a gene that stimulates and/or stabilizes the formation of Tregs. In some embodiments, the gene that stimulates and/or stabilizes the formation of Tregs is encoded by the same recombinant nucleic acid molecule as the recombinant nucleic acid molecule encoding the TFP. In some embodiments, the gene that stimulates and/or stabilizes the formation of Tregs is encoded by a different recombinant nucleic acid molecule than the recombinant nucleic acid molecule encoding the TFP. In some embodiments, the gene that stimulates and/or stabilizes the formation of Tregs is FOXP3, HELIOS, BACH2, or pSTAT5. In some embodiments, the Treg further comprises a switch receptor. In some embodiments, the switch receptor is encoded by the same recombinant nucleic acid molecule as the recombinant nucleic acid molecule encoding the TFP. In some embodiments, the switch receptor is encoded by a different recombinant nucleic acid molecule than the recombinant nucleic acid molecule encoding the TFP. In some embodiments, the switch receptor is an IL7-IL2 switch receptor, an IL7-IL10 switch receptor, or a TNF-alpha-IL2 switch receptor.

In some embodiments, the Treg comprises more than one gene that stimulates and/or stabilizes the formation of Tregs and/or more than one switch receptor. In some embodiments, the expression of one or more of PKC theta, STUB1, and CCAR2 in the Treg cell is reduced or eliminated. In some embodiments, the expression of one or more of CDK8 and CDK19 reduced, deleted, or pharmacologically inhibited to stabilized Treg formation.

In some embodiments, the peptide of the peptide-MHC complex is an autoantigen or a fragment thereof. In some embodiments, the peptide of the peptide-MHC complex is an exogenous antigen or a fragment thereof. In some embodiments, the binding domain comprises an antigen binding domain. In some embodiments, the antigen binding domain comprises an autoantigen binding domain or an exogenous antigen binding domain. In some embodiments, the autoantigen binding domain specifically binds an autoantigen.

In some embodiments, the exogenous antigen binding domain specifically binds an exogenous antigen. In some embodiments, the autoantigen is one or more of islet glucose-6-phosphatase catalytic subunit related protein (IGRP), insulin, HLA-A2, myelin, or alpha-gliadin or a fragment thereof. In some embodiments, the exogenous antigen is FVIII or a therapeutic macromolecule, e.g., a therapeutic polypeptide, or a fragment thereof.

In some embodiments, the antigen binding domain binds to a cell membrane associated antigen. In some embodiments, the antigen binding domain binds to a circulating antigen. In some embodiments, the antigen binding domain is specific to an antigen on an islet cell. In some embodiments, the antigen binding domain is an antibody or functional fragment thereof. In some embodiments, the antibody or functional fragment thereof is an scFv or a single domain antibody.

In some embodiments, the antibody or functional fragment thereof is human or humanized. In some embodiments, the binding domain is a TCR mimic, e.g., specifically binds a peptide-MHC-complex.

In some embodiments, the T regulatory cell reduces cytokine production of an effector T cell having the antigen, the MHC-peptide complex, or the T cell receptor that specifically binds the MHC-peptide complex, relative to a T regulatory cell having a Treg that does not contain the TFP. In some embodiments, the Treg is a CD4+ CD25+ FoxP3+ Treg or a CD8+ regulatory T cell.

In some embodiments, the intracellular signaling domain is selected from the group consisting of CD3 gamma, CD3 delta, CD3 epsilon, and CD3 zeta. In some embodiments, the TCR-integrating subunit comprises (i) a TCR extracellular domain, (ii) a TCR transmembrane domain, and (iii) a TCR intracellular domain, wherein at least two or three of (i), (ii), and (iii) are from the same TCR subunit.

In some embodiments, the binding domain is operatively linked to the TCR extracellular domain by a linker sequence. In some embodiments, the linker sequence comprises (G4S)n, wherein n=1 to 4.

In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit and functional fragments thereof.

In some embodiments, the TFP includes a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, and functional fragments thereof.

In some embodiments, the TFP includes a TCR intracellular domain of a protein selected from the group consisting of TCR alpha, TCR beta, TCR gamma, TCR delta. a CD3 epsilon TCR subunit, a CD3 gamma TCR subunit, and a CD3 delta TCR subunit.

In some embodiments, the TFP comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d and functional fragments thereof. In some embodiments, the ITAM replaces an ITAM of CD3 gamma, CD3 delta, or CD3 epsilon. In some embodiments, the Treg is autologous. In some embodiments, the Treg is allogeneic.

In another aspect, the present disclosure provides a pharmaceutical composition comprising the T regulatory cell described herein, and a pharmaceutically acceptable carrier.

In another aspect, the present disclosure provides a recombinant nucleic acid comprising a sequence encoding the TFP of the T regulatory cell described herein. In some embodiments, the nucleic acid is selected from the group consisting of a DNA and an RNA. In some embodiments, the nucleic acid is an mRNA. In some embodiments, the nucleic acid is circRNA. In some embodiments, the recombinant nucleic acid comprises a nucleic acid analog, wherein the nucleic acid analog is not in an encoding sequence of the recombinant nucleic acid. In some embodiments, the recombinant nucleic acid further comprises a leader sequence. In some embodiments, the recombinant nucleic acid further comprises a promoter sequence. In some embodiments, the recombinant nucleic acid further comprises a sequence encoding a poly(A) tail. In some embodiments, the recombinant nucleic acid further comprises a 3′UTR sequence. In some embodiments, the nucleic acid is an isolated nucleic acid or a non-naturally occurring nucleic acid. In some embodiments, the nucleic acid is an in vitro transcribed nucleic acid.

In another aspect, the present disclosure provides a vector comprising the recombinant nucleic acid described herein. In some embodiments, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some embodiments, the vector is an in vitro transcribed vector.

In another aspect, the present disclosure provides a circular RNA comprising the recombinant nucleic acid described herein.

In another aspect, the present disclosure provides a method of treating or preventing a disease or disorder comprising administering to a subject in need thereof a therapeutically effective amount of the T regulatory cell described herein. In some embodiments, the disease or disorder is an autoimmune disease. In some embodiments, the autoimmune disease is an autoantibody-mediated autoimmune disease. In some embodiments, the autoimmune disease is selected from the group comprising multiple sclerosis, autoimmune hemolytic anemia, celiac disease, and chronic inflammatory demyelinating polyradiculoneuropathy. In some embodiments, the disease or disorder is inflammation, e.g., an inflammatory disease or disorder, an allergic reaction, or transplant rejection.

In another aspect, the present disclosure provides a composition for use in treating or preventing a disease or disorder in a subject in need thereof, comprising the recombinant nucleic acid described herein or the T regulatory cell described herein. In some embodiments, the disease or disorder is an autoimmune disease. In some embodiments, the disease or disorder is inflammation, e.g., an inflammatory disease or disorder, an allergic reaction, or transplant rejection. In some embodiments, the subject has or is at risk of developing an autoimmune disease, inflammation, e.g., an inflammatory disease or disorder, an allergic reaction, or transplant rejection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic of engineered regulatory T cell (Treg) mediated T cell response after recognizing the cell-membrane associated or circulating autoantigen(s). The Treg shown here can express a T-cell receptor (TCR) fusion protein comprising a binding domain that targets the cell-membrane associated or circulating autoantigen(s).

FIG. 2 shows a schematic of the process for producing TFP-expressing Treg.

FIG. 3 shows a schematic of the process for isolating Treg.

FIG. 4 shows a schematic for the process of producing TFP-expressing Treg and CD4+ cells and a graph showing the expansion of untraduced Tregs, HLA-A2 TFP Tregs, HLA-A2 TFP-2A-FoxP3 Tregs, untransduced CD4+ T cells, HLA-A2 TFP CD4+T cells, and HLA-A2 TFP-2A-FoxP3 CD4+ T cells when produced according to the protocol shown.

FIGS. 5A and 5B are a series of plots showing expression of FoxP3, CD25, Helios, and CD4 in untraduced Tregs, HLA-A2 TFP Tregs, HLA-A2 TFP-2A-FoxP3 Tregs, HLA-A2 TFP CD4+T cells, and HLA-A2 TFP-2A-FoxP3 CD4+T cells. FIG. 5A shows FoxP3, CD25, and Helios in cells from Donor 1. FIG. 5B shows FoxP3, CD25, Helios, CD4, and CD25 in cells from Donor 2.

FIG. 6 shows a schematic of an antigen independent suppression assay.

FIGS. 7A and 7B are a series of graphs showing cytokine expression of unstimulated polyclonal CD4+ and CD8+ T cells, stimulated polyclonal CD4+ and CD8+ T cells alone or mixed with untraduced Tregs, HLA-A2 TFP Tregs, HLA-A2 TFP-2A-FoxP3 Tregs, or HLA-A2 TFP-2A-FoxP3 CD4+T cells in the antigen independent suppression assay shown in FIG. 6. FIG. 7A shows data from Donor 1. FIG. 7B shows data from Donor 2.

FIGS. 8A and 8B are a series of graphs showing suppression of expansion of stimulated polyclonal CD4+ T cells, CD8+ T cells, and CD3+ T cells mixed with untraduced Tregs, HLA-A2 TFP Tregs, HLA-A2 TFP-2A-FoxP3 Tregs, or HLA-A2 TFP-2A-FoxP3 CD4+T cells in the antigen independent suppression assay shown in FIG. 6. FIG. 8A shows data from Donor 1.

FIG. 8B shows data from Donor 2.

FIG. 9 is a series of plots showing expression of MH1-TFP and CD4 in untraduced Tregs, MH1 TFP Tregs, MH1 TFP-2A-FoxP3 Tregs, untransduced CD4+ T cells, MH1 TFP CD4+T cells, and MH1 TFP-2A-FoxP3 CD4+T cells.

FIG. 10 is a series of plots showing expression of FoxP3, CD25, and Helios in MH1 TFP Tregs, MH1 TFP-2A-FoxP3 Tregs, MH1 TFP CD4+T cells, and MH1 TFP-2A-FoxP3 CD4+T cells.

FIG. 11 shows a schematic of an antigen dependent suppression assay.

FIG. 12 is a series of graphs showing cytokine expression of MH1 TFP effector T cells alone or contacted with MSTO-msln cells only, or contacted with MSTO-msln cells and mixed with MH1 TFP Tregs, MH1 TFP-2A-FoxP3 Tregs or and MH1 TFP-2A-FoxP3 CD4+T cells in the antigen dependent suppression assay shown in FIG. 11.

FIG. 13 is a series of graphs showing suppression of expansion of MH1 TFP effector T cells contacted with MSTO-msln cells and mixed with MH1 TFP Tregs, MH1 TFP-2A-FoxP3 Tregs or and MH1 TFP-2A-FoxP3 CD4+T cells in the antigen dependent suppression assay shown in FIG. 11.

FIG. 14 shows a schematic of an antigen dependent suppression assay (e.g., mixed lymphocyte reaction or MLR) with HLA-A2 TFP regulatory T cells.

FIG. 15 is a series of graphs showing cytokine expression of effector T cells alone or with HLA matched or mismatched dendritic cells, or cocultured with mismatched dendritic cells and untraduced Tregs, HLA-A2 TFP Tregs, HLA-A2 TFP-2A-FoxP3 Tregs, or HLA-A2 TFP-2A-FoxP3 CD4+T cells in the antigen dependent suppression assay shown in FIG. 14.

FIG. 16 is a series of graphs showing suppression of expansion of effector T cells when cocultured with mismatched dendritic cells and untraduced Tregs, HLA-A2 TFP Tregs, HLA-A2 TFP-2A-FoxP3 Tregs, or HLA-A2 TFP-2A-FoxP3 CD4+T cells in the antigen dependent suppression assay shown in FIG. 14.

FIG. 17 is a series of plots showing expression of FoxP3, CD25, and Helios in Tregs isolated according to the methods described in Example 5 prior to transduction.

FIG. 18 is a series of plots showing expression of FoxP3, CD25, Helios, CD4, and CD25 in untraduced Tregs, HLA-A2 TFP Tregs, HLA-A2 TFP-2A-FoxP3 Tregs, and HLA-A2 TFP CD4+T cells prepared according to the protocol described in Example 5.

FIG. 19 is a graph showing the expansion of untraduced Tregs, HLA-A2 TFP Tregs, HLA-A2 TFP-2A-FoxP3 Tregs, untransduced CD4+ T cells, HLA-A2 TFP CD4+T cells, and HLA-A2 TFP-2A-FoxP3 CD4+T cells when prepared according to the protocol described in Example 5.

### DETAILED DESCRIPTION OF THE INVENTION

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising,” is inclusive and does not exclude additional, unrecited integers or method/process steps.

The term “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein. In some embodiments, the subject has autoimmune diseases described herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing autoimmune diseases is treated with the methods of the present disclosure and does not later develop the autoimmune diseases, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

As is used herein, the terms “T-cell receptor” and “T-cell receptor complex” are used interchangeably to refer to a molecule found on the surface of T cells that is, in general, responsible for recognizing antigens. The TCR comprises a heterodimer consisting of an alpha and beta chain in 95% of T cells, whereas 5% of T cells have TCRs consisting of gamma and delta chains. The TCR further comprises a combination of components of the CD3 complex. In some embodiments, the TCR comprises CD3ε. In some embodiments, the TCR comprises CD3γ. In some embodiments, the TCR comprises CD3ζ. In some embodiments, the TCR comprises CD3δ. Engagement of the TCR with antigen, e.g., with antigen and MHIC, results in activation of its T cells through a series of biochemical events mediated by associated enzymes, co-receptors, and specialized accessory molecules.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHIC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHIC's) on its surface. T cells may recognize these complexes using their T cell receptors (TCRs). APCs process antigens and present them to T cells.

“Major histocompatibility complex (MHC) molecules are typically bound by TCRs as part of peptide:MHIC complex. The MHIC molecule may be an MHIC class I or II molecule. The complex may be on the surface of an antigen presenting cell, such as a dendritic cell or a B cell, or any other cell, or it may be immobilized by, for example, coating on to a bead or plate. The human leukocyte antigen system (HLA) is the name of the gene complex which encodes major histocompatibility complex (MHC) in humans and includes HLA class I antigens (A, B & C) and HLA class II antigens (DP, DQ, & DR). HLA alleles A, B and C present peptides derived mainly from intracellular proteins, e.g., proteins expressed within the cell. During T cell development in vivo, T cells undergo a positive selection step to ensure recognition of self MHCs followed by a negative step to remove T cells that bind too strongly to MHC which present self-antigens. As a consequence, certain T cells and the TCRs they express will only recognize peptides presented by certain types of MHC molecules—i.e. those encoded by particular HLA alleles. This is known as HLA restriction. One HLA allele can be HLA-A*0201 (HLA-A2), which is expressed in the vast majority (>50%) of the Caucasian population. Accordingly, TCRs which bind WT1 peptides presented by MHC encoded by HLA-A*0201 (i.e. are HLA-A*0201 restricted) are advantageous since an immunotherapy making use of such TCRs will be suitable for treating a large proportion of the Caucasian population. Other HLA-A alleles of interest can include HLA-A*0101, HLA-A*2402, and HLA-A*0301. Widely expressed HLA-B alleles of interest can include HLA-B*3501, HLA-B*0702 and HLA-B*3502.

As is used herein, the “MHC-peptide complex” comprises a MHC molecule or fraction thereof, e.g., a MHC class I or II molecule, and a peptide, e.g., an epitope. In some embodiments, the peptide is an antigen, e.g., autoantigen or exogenous antigen, or fragment thereof that results after processing by an antigen presenting cell (APC).

The term “intracellular signaling domain,” as used herein, refers to an intracellular portion of a molecule. The intracellular signaling domain generates a signal that promotes an immune effector function of the TFP containing cell, e.g., a modified T-T cell. Examples of immune effector function, e.g., in a modified T-T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that may be needed for an efficient immune response. Costimulatory molecules include, but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

The term “antibody,” as used herein, refers to a protein, or polypeptide sequences derived from an immunoglobulin molecule, which specifically binds to an antigen. Antibodies can be intact immunoglobulins of oligoclonal or monoclonal origin, or fragments thereof and can be derived from natural or from recombinant sources.

The terms “antibody fragment” refers to at least one portion of an antibody, or recombinant variants thereof, that contains the antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs. A camelid “V_{H}H” domain is a heavy chain comprising a single variable antibody domain.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “functional disruption” refers to a physical or biochemical change to a specific (e.g., target) nucleic acid (e.g., gene, RNA transcript, of protein encoded thereby) that prevents its normal expression and/or behavior in the cell. In one embodiment, a functional disruption refers to a modification of the gene via a gene editing method. In one embodiment, a functional disruption prevents expression of a target gene (e.g., an endogenous gene).

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like.

Nonclinical types of lentiviral vectors are also available and would be known to one skilled in the art.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the present disclosure can be replaced with other amino acid residues from the same side chain family and the altered TFP can be tested using the functional assays described herein.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which can be used for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the present disclosure are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner present in a sample, but which does not necessarily and substantially recognize or bind other molecules in the sample.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Overview

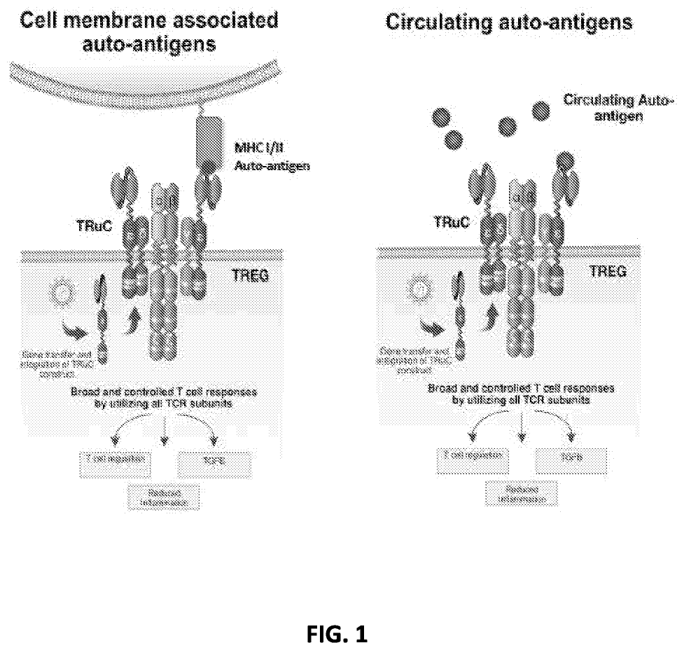

The present disclosure provides regulatory T cells (Tregs) expressing T-cell receptor (TCR) fusion proteins (TFPs). These cells can express a T cell receptor fusion construct that recognizes an antigen, e.g., an autoantigen, allergen, or transplanted tissue antigen. In some embodiments, the T cell receptor fusion construct recognizes an exogenous antigen, e.g., a therapeutic. In some instances, the T cell receptor fusion construct recognizes a T cell receptor. In some embodiments, the T cell receptor fusion construct recognizes a peptide-MHC complex. Upon activation, TFP-expressing Tregs can function to reduce autoimmune reactions, allergic responses, transplant rejection, or immune responses to therapeutics. They may be applied to the treatment of numerous inflammatory conditions not limited to autoimmunity, allergy, transplantation, and immune responses to therapeutics. FIG. 1 shows a schematic of engineered regulatory T cell (Treg) mediated T cell response after recognizing the cell-membrane associated or circulating autoantigen(s).

Tregs can be essential to maintain self-tolerance and dampen immune responses during infection. Some subsets of Tregs can be characterized by high expression of CD25 and FOXP3, the master-regulator of their phenotype and suppressive function. The role of FOXP3 in controlling Treg development and function can be illustrated by the study of Tregs from patients with immunodysregulation polyendocrinopathy enteropathy X-linked (IPEX) syndrome. Depending on the specific mutation, IPEX patients may or may not have circulating FOXP3⁺ T cells, but even if FOXP3⁺ T cells may be present, they may be functionally defective due to inadequate FOXP3 transcriptional function.

Mechanistically, Tregs can suppress the proliferation and function of many immune cells, even at very low Treg:effector cell ratios. In terms of suppressive pathways, multiple possibilities can happen, such as immunosuppressive cytokines, contact-dependent cytotoxicity, metabolic disruption, and suppression of antigen presenting cells via co-inhibitory molecule expression. Focusing on human Tregs, there may be a dominant role for CTLA-4 and TGF-β. Monogenic mutations affecting CTLA-4 or proteins in its pathway can affect Treg function and antibodies that block activation of TGF-β by human Tregs can prevent their ability to control xenogeneic graft-versus-host disease (GVHD). An additional aspect of Treg mechanisms can be their ability to take on characteristics of other T helper (T_{H}) cells, resulting in sub-specialization and enhanced suppression of the T_{H} cell subset they mirror. These sub-specialized Tregs may have unique suppressive mechanisms or may be able to traffic to the relevant sites of inflammation more efficiently.

The immunosuppressive properties of Tregs can make them attractive candidates for cellular therapy, particularly for application in conditions such as hematopoietic stem cell transplantation (HSCT), solid organ transplantation, and autoimmunity. However, harnessing Tregs for this purpose may not be trivial due to limitations related to cell isolation and expansion. Attempts to “boost” Tregs in vivo can be made using low doses of IL2 alone or in combination with other agents. However, these approaches can lead to mixed results, in part due to the pleiotropic roles of IL2. An alternate to in vivo-boosting can be adoptive therapy with ex vivo-enriched, often expanded, Tregs. This method may overcome defective or low numbers of Tregs by transfer of a large number of Tregs to re-set the Treg:Tconv cell balance. There remains numerous barriers to use engineered Treg cells in the clinic. These can include the ability to generate enough Treg cells using current technologies, maintaining Treg function after expansion and finally arming Tregs with appropriate targeting tools. The engineered Tregs described herein can overcome many of these barriers.

The engineered Tregs described herein can overcome difficulties in obtaining sufficient numbers of Tregs. The ability to generate enough Tregs may be hampered by the need to isolate endogenous Tregs (which may account for less than 2% of circulating T cells) and then expand them. The methods described herein can engineer all T cells to become Tregs during an ex-vivo process. Additionally, or alternatively, the methods described herein engineer Treg to maintain their regulatory state. These engineered cells can be expanded in vitro and re-introduced to patients. This can obviate the need to isolate endogenous Tregs.

The engineered T regs described herein can maintain Treg function. The engineered T cells can encompass and express a recombinant nucleic acid molecule encoding a T-cell receptor-integrating (TCR) fusion protein (TFP). The engineered T cells described herein can be referred to as TFP-Tregs. Unlike CAR-Treg therapies, TFP-Tregs described herein can signal through the TCR which can be for long term Treg function. This characteristic of TFP-Tregs can be expected to support long term function after transfer. The engineered Tregs described herein can also express Forkhead FOXP3, HELIOS, BACH2, or pSTAT5, which can assist in maintaining Treg function after transfer. The engineered TFP-Tregs can express an IL7-IL2 switch receptor, an IL7-IL10 switch receptor, or a TNF-alpha-IL2 switch receptor, which can assist in maintaining Treg function after transfer. This switch receptor may assist in the expansion of Tregs in vitro

The engineered Tregs described herein can have optimized Treg targeting. Both cell expressed (e.g., myelin on neurons in Multiple Sclerosis, collagen in joints in Rheumatoid Arthritis) and circulating autoantigens (e.g., gliadin in celiac disease) can be targeted by TFP-Tregs.

The engineered Treg described herein can comprise: (a) a recombinant nucleic acid molecule encoding a T cell receptor (TCR) fusion protein (TFP) comprising (i) a TCR-integrating subunit comprising (1) an extracellular domain, (2) a TCR transmembrane domain, and (3) a TCR intracellular domain; and; (ii) a binding domain. The intracellular domain may comprise a stimulatory domain from an intracellular signaling domain. The TCR-integrating subunit and the binding domain can be operatively linked. The TFP can functionally interact with an endogenous TCR when expressed in a T cell.

The engineered Treg can be made into a pharmaceutical composition comprising a pharmaceutically acceptable carrier and be used to treat a disease such as an autoimmune disease.

### T-cell Receptor (TCR) Fusion Protein

The present disclosure provides recombinant nucleic acid constructs encoding T-cell receptor (TCR) fusion proteins (TFPs) and variants thereof. The TFP can comprise a binding domain, e.g., an antibody or antibody fragment that binds specifically to an autoimmune disease related antigen (autoantigen), to a therapeutic, to a TCR that specifically binds an autoantigen or therapeutic, a ligand, e.g., a peptide-MHC complex, or a ligand binding protein, e.g., a T cell receptor mimic, wherein the sequence of the binding domain is contiguous with and in the same reading frame as a nucleic acid sequence encoding a TCR-integrating subunit or portion thereof. The TFPs provided herein are able to associate with one or more endogenous (or alternatively, one or more exogenous, or a combination of endogenous and exogenous) TCR subunits in order to form a functional TCR complex. The present disclosure also provides a TFP molecule or a TCR complex having the TFP molecule incorporated therein. The present disclosure also provides a cell (e.g., a T cell or a Treg) comprises the TFP or the recombinant nucleic acid molecule encoding the TFP. Advantageously, such TFPs, when expressed in a T-cell, can target T cells having T cell receptors that are specific for autoantigens or therapeutics. When administered to a subject, such TFP-expressing cells can treat autoimmune diseases, inflammatory diseases or disorders, allergic reactions, transplant rejections, or immune responses to therapeutics.

The TFP can comprise a TCR-integrating subunit comprising (1) an extracellular domain, (2) a TCR transmembrane domain, and (3) a TCR intracellular domain. The intracellular signaling domain can be selected from CD3 gamma, CD3 delta, CD3 epsilon, CD3 zeta, TCR alpha, TCR beta, TCR gamma, and TCR delta. The TCR-integrating subunit can comprise (i) a TCR extracellular domain, (ii) a TCR transmembrane domain, and (iii) a TCR intracellular domain. At least two of the TCR extracellular domain, the TCR transmembrane domain, and the TCR intracellular domain can be derived from the same TCR subunit (e.g., CD3 gamma, CD3 delta, CD3 epsilon, CD3 zeta, TCR alpha, TCR beta, TCR gamma, or TCR delta). The TCR extracellular domain, the TCR transmembrane domain, and the TCR intracellular domain can be derived from the same TCR subunit. The TCR-integrating subunit can comprise a full-length CD3 gamma, CD3 delta, CD3 epsilon, CD3 zeta, TCR alpha, TCR beta, TCR gamma, or TCR delta. The TCR-integrating subunit can comprise a full-length transmembrane domain derived from CD3 gamma, CD3 delta, CD3 epsilon, CD3 zeta, TCR alpha, TCR beta, TCR gamma, or TCR delta. The TCR-integrating subunit can comprise a full-length extracellular domain derived from CD3 gamma, CD3 delta, CD3 epsilon, CD3 zeta, TCR alpha, TCR beta, TCR gamma, or TCR delta. The TCR-integrating subunit can comprise a full-length intracellular domain derived from CD3 gamma, CD3 delta, CD3 epsilon, CD3 zeta, TCR alpha, TCR beta, TCR gamma, or TCR delta. The encoded binding domain can be connected to the TCR extracellular domain by a linker sequence. In some embodiments, the encoded linker sequence comprises (G4S)n, wherein n=1 to 4.

The TFP can include an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

The encoded TFP can include a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, a CD3 zeta TCR subunit, functional fragments thereof, and amino acid sequences thereof having at least one but not more than 20 modifications.

The TFP can include an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the ITAM replaces an ITAM of CD3 gamma, CD3 delta, or CD3 epsilon.

In one aspect, the TFPs of the present disclosure comprise a target-specific binding element otherwise referred to as a binding domain. The choice of moiety can depend upon the type and number of target antigen that define the surface of a target cell. For example, the binding domain may be chosen to recognize a target antigen that acts as a cell surface marker on target cells associated with a particular disease state. Thus, examples of cell surface markers that may act as target antigens for the binding domain in a TFP of the present disclosure include those associated with viral, bacterial and parasitic infections; autoimmune diseases; and cancerous diseases (e.g., malignant diseases).

### Binding Domain

Provided herein are compositions comprising a TFP comprising a binding domain, wherein the binding domain targets the TFP to a T cell having a TCR that binds an autoantigen or therapeutic. The binding domain can be an antigen binding domain, e.g., an autoantigen or exogenous antigen binding domain, a T cell receptor ligand, e.g., a peptide-MHC complex, or a T cell receptor mimic, e.g., an antibody that binds the peptide-MHIC complex. In some embodiments, the autoantigen is associated with an autoimmune disease or inflammation. In some embodiments, the autoantigen is associated with transplant rejection.

In one aspect, the TFP-mediated T cell response can be directed to an antigen of interest by way of engineering an antigen-binding domain into the TFP that specifically binds a desired antigen. In some embodiments, the antigen of interest is a peptide-MHIC complex and the antigen binding domain is a T cell receptor mimic.

The antigen binding domain can be any domain that binds to the antigen including but not limited to a monoclonal antibody, a polyclonal antibody, a recombinant antibody, a human antibody, a humanized antibody, a murine antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise, a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

A humanized antibody or antibody fragment may retain a similar antigenic specificity as the original antibody, e.g., in the present disclosure, the ability to bind a human autoantigen. In some embodiments, a humanized antibody or antibody fragment may have improved affinity and/or specificity of binding to the target antigen.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

In one aspect, the present disclosure contemplates modifications of the starting antibody or fragment (e.g., scFv) amino acid sequence that generate functionally equivalent molecules. For example, the V_{H} or V_{L} of a binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of an antigen binding domain, e.g., scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting TFP construct.

Exemplary autoantigens include, but are not limited to, Gliandin, islet glucose-6-phosphatase catalytic subunit related protein (IGRP), ChgA, IAPP, peripherin tetraspabin-7 P4Hb, GRP78, urocortin-3, insulin gene enhancer protein isl-1, insulin, desmogelin 1, desmogelin 3, GAD65, IA-2, ZnT8, collagen type II, human chondrocyte glycoprotein 39, proteoglycans, citrullinated filaggrin, tryptase, heterogeneous nuclear ribonucleoprotein A2/B1, aldolase, α-enolase, calreticulin, 60 kDa heat shock protein (HSP60), stress-induced phosphoprotein 1, phosphoglycerate kinase 1 (PGK1), far upstream element-binding proteins (FUSE-BP) 1 and 2, quaporin-4 water channel (AQP4), Hu, Ma2, collapsin response-mediator protein 5 (CRMP5), and amphiphysin, voltage-gated potassium channel (VGKC), N-methyl-d-aspartate receptor (NMDAR), α-amino-3-hydroxy-5-methyl-4-isoxazoleproprionic acid (AMPAR), Thyroid peroxidase, thyroglobulin, Anti-N-methyl-D-aspartate receptor (NR1 subunit), Rh blood group antigens, I antigen, Dsgl/3, BP180, BP230, Acetylcholine nicotinic postsynaptic receptors, thyrotropin receptors, platelet integrin, GpIIbTIIa, Collagen alpha-3(IV) chain, rheumatoid factor, calpastatin, citrullinated proteins, Myelin basic protein (MBP), Myelin oligodendrocyte glycoprotein (MOG) peptides, alpha-beta-crystallin, DNA, histone, ribosomes, R P, tissue transglutaminase (TG2), intrinsic factor, 65-kDa antigen, phosphatidylserine, ribosomal phosphoproteins, anti-neutrophil cytoplasmic antibody, Scl-70, Ul-R P, ANA, SSA, anti-SSB, antinuclear antibodies (ANA), antineutrophil cytoplasm antibodies (ANCA), Jo-1, antimitochondrial antibodies, gp210, p62, splOO, antiphospho lipid antibodies, U1-70 kd snRNP, GQlb ganglioside, GM1, asialo GM1, GDlb, anti-smooth muscle antibodies (ASMA), anti-liver-kidney microsome-1 antibodies (ALKM-1), anti-liver cytosol antibody-1 (ALC-1), IgA antiendomysial antibodies, neutrophil granule proteins, streptococcal cell wall antigen, intrinsic factor of gastric parietal cells, insulin (IAA), glutamic acid decarboxylase (GAA or GAD), protein tyrosine phosphatase (IA2 or ICA512), PLA2R1, THSD7A1, HLA-A2, CEA, FVIII, GAD65, and citrullinated Vimentin. Exemplary antigen binding domains that bind autoantigens include, e.g., anti-HLA-A2 antibodies. Exemplary HLA-A2 antibodies include, e.g., 3PF12, the BB7.2 scFv described in MacDonald et al., (J Clin Invest. 2016 Apr. 1; 126(4): 1413-1424), those described in A. Martin et al., Cytotherapy, Dec. 9, 2020, and those described in United States Patent Application Nos. 20200283530 and 20200283529, each of which is incorporated by reference herein.

In some embodiments, the antigen is an exogenous antigen, e.g., a therapeutic, e.g., a therapeutic that induces an immune response in a subject. Exemplary therapeutics that may induce an immune response include, but are not limited to, infusible therapeutic proteins, enzymes, enzyme cofactors, hormones, blood clotting factors, cytokines and interferons, growth factors, monoclonal antibodies, and polyclonal antibodies (e.g., that are administered to a subject as a replacement therapy), and proteins associated with Pompe's disease (e.g., alglucosidase alfa, rhGAA (e.g., Myozyme and Lumizyme (Genzyme)). Therapeutic proteins also include proteins involved in the blood coagulation cascade. Therapeutic proteins include, but are not limited to, Factor VIII, Factor VII, Factor IX, Factor V, von Willebrand Factor, von Heldebrant Factor, tissue plasminogen activator, insulin, growth hormone, erythropoietin alfa, VEGF, thrombopoietin, lysozyme, antithrombin and the like. Therapeutic proteins also include adipokines, such as leptin and adiponectin. Other examples of therapeutic proteins are as described below and elsewhere herein. Also included are fragments or derivatives of any of the therapeutic proteins provided as the antigen.

Examples of therapeutic proteins used in enzyme replacement therapy of subjects having a lysosomal storage disorder include, but are not limited to, imiglucerase for the treatment of Gaucher's disease (e.g., CEREZYME™), α-galactosidase A (a-gal A) for the treatment of Fabry disease (e.g., agalsidase beta, FABRYZYME™), acid a-glucosidase (GAA) for the treatment of Pompe disease (e.g., alglucosidase alfa, LUMIZYME™, MYOZYME™), arylsulfatase B for the treatment of Mucopolysaccharidoses (e.g., laronidase, ALDURAZYME™, idursulfase, ELAPRASE™, arylsulfatase B, NAGLAZYME™).

Examples of enzymes include oxidoreductases, transferases, hydrolases, lyases, isomerases, and ligases.

Examples of hormones include Melatonin (N-acetyl-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerian hormone (or mullerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

Examples of blood and blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, prekallikrein (Fletcher factor), high-molecular weight kininogen (HMWK) (Fitzgerald factor), fibronectin, fibrin, thrombin, antithrombin III, heparin cofactor II, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumour necrosis factor-alpha (TNF-α), Vascular endothelial growth factor (VEGF), Wnt Signaling Pathway, placental growth factor (PlGF), [(Foetal Bovine Somatotrophin)] (FBS), IL-1, IL-2, IL-3, IL-4, IL-5, IL-6, and IL-7.

Examples of monoclonal antibodies include Abagovomab, Abciximab, Adalimumab, Adecatumumab, Afelimomab, Afutuzumab, Alacizumab pegol, ALD, Alemtuzumab, Altumomab pentetate, Anatumomab mafenatox, Anrukinzumab, Anti-thymocyte globin, Apolizumab, Arcitumomab, Aselizumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Brentuximab vedotin, Briakinumab, Canakinumab, Cantuzumab mertansine, Capromab pendetide, Catumaxomab, Cedelizumab, Certolizumab pegol, Cetuximab, Citatuzumab bogatox, Cixutumumab, Clenoliximab, Clivatuzumab tetraxetan, Conatumumab, Dacetuzumab, Daclizumab, Daratumumab, Denosumab, Detumomab, Dorlimomab aritox, Dorlixizumab, Ecromeximab, Eculizumab, Edobacomab, Edrecolomab, Efalizumab, Efungumab, Elotuzumab, Elsilimomab, Enlimomab pegol, Epitumomab cituxetan, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumab ozogamicin, GC1008, Girentuximab, Glembatumumab vedotin, Golimumab, Gomiliximab, Ibalizumab, Ibritumomab tiuxetan, Igovomab, Imciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Olaratumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, Rafivirumab, Ramucirumab, Ranibizumab, Raxibacumab, Regavirumab Reslizumab, Rilotumumab, Rituximab, Robatumumab, Rontalizumab, Rovelizumab, Ruplizumab, Satumomab pendetide, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, Ticilimumab (tremelimumab), Tigatuzumab, Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab aritox.

Examples of infusion therapy or injectable therapeutic proteins include, for example, Tocilizumab (ROCHE/ACTEMRA®), alpha-1 antitrypsin (Kamada/AAT), HEMATIDE® (Affymax and Takeda, synthetic peptide), albinterferon alfa-2b (NOVARTIS/ZALBIN™) RHUCIN® (Pharming Group, C1 inhibitor replacement therapy), tesamorelin (Theratechnologies/Egrifta, synthetic growth hormone-releasing factor), ocrelizumab (Genentech, Roche and Biogen), belimumab (GLAXOSMITHKLINE/BENLYSTA®), pegloticase (SAVIENT PHARMACEUTICALS/KRYSTEXX™), taliglucerase alfa (Protalix/Uplyso), agalsidase alfa (SHIRE/REPLAGAL®), velaglucerase alfa (Shire).

In some embodiments, the binding domain comprises an antibody or fragment thereof that is a TCR mimic, e.g., binds a MHC-peptide complex.

In some embodiments, the binding domain comprises a MHC-peptide complex.

In some embodiments, the peptide of the MHC-peptide complex comprises any of the autoantigens or exogenous antigens described herein or fragments thereof, e.g., fragments that would be generated by APCs.

### Extracellular Domain

The extracellular domain of the TFP may be derived either from a natural or from a recombinant source. Where the source is natural, the domain may be derived from any protein, but in particular a membrane-bound or transmembrane protein. In one aspect, the extracellular domain is capable of associating with the transmembrane domain. An extracellular domain of particular use in this present disclosure may include at least the extracellular region(s) of e.g., the alpha, beta, gamma, delta, or zeta chain of the T-cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154.

In some embodiments, the extracellular domain is a TCR extracellular domain. In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a TCR alpha chain, a TCR beta chain, a TCR delta chain, or a TCR gamma chain. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a TCR alpha chain, a TCR beta chain, a TCR delta chain, or a TCR gamma chain. In some embodiments, the extracellular domain comprises a sequence encoding the extracellular domain of a TCR alpha chain, a TCR beta chain, a TCR delta chain, or a TCR gamma chain having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of an IgC domain of TCR alpha, a TCR beta, a TCR delta, or a TCR gamma. In some embodiments, the extracellular domain comprises a sequence having at least about 50%, 55%, 60%, 65%, 70%, 75%, 80%, 85%, 90%, 95%, 98%, 99% or more sequence identity to a sequence encoding an IgC domain of TCR alpha, a TCR beta, a TCR delta, or a TCR gamma. In some embodiments, the extracellular domain comprises a sequence encoding an IgC domain of TCR alpha, TCR beta, TCR delta, or TCR gamma having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a CD3 epsilon TCR subunit, a CD3 gamma TCR subunit, or a CD3 delta TCR subunit. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a CD3 epsilon TCR subunit, a CD3 gamma TCR subunit, or a CD3 delta TCR subunit. In some embodiments, the extracellular domain comprises a sequence encoding the extracellular domain of a CD3 epsilon TCR subunit, a CD3 gamma TCR subunit, or a CD3 delta TCR subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

The extracellular domain can be a TCR extracellular domain. The TCR extracellular domain can be derived from a TCR alpha chain, a TCR beta chain, a TCR gamma chain, a TCR delta chain, a CD3 epsilon TCR subunit, a CD3 gamma TCR subunit or a CD3 delta TCR subunit. The extracellular domain can be a full-length TCR extracellular domain or fragment (e.g., functional fragment) thereof. The extracellular domain can comprise a variable domain of a TCR alpha chain, a TCR beta chain, a TCR gamma chain or a TCR delta chain. The extracellular domain can comprise a variable domain and a constant domain of a TCR alpha chain, a TCR beta chain, a TCR gamma chain or a TCR delta chain. In some cases, the extracellular domain may not comprise a variable domain.

The extracellular domain can comprise a constant domain of a TCR alpha chain, a TCR beta chain, a TCR gamma chain or a TCR delta chain. The extracellular domain can comprise a full-length constant domain of a TCR alpha chain, a TCR beta chain, a TCR gamma chain or a TCR delta chain. The extracellular domain can comprise a fragment (e.g., functional fragment) of the full-length constant domain of a TCR alpha chain, a TCR beta chain, a TCR gamma chain or a TCR delta chain. For example, the extracellular domain can comprise at least about 5, 10, 15, 20, 25, 30, 35, 40, 45, 50, 55, 60, 65, 70, 75, 80, 85, 90, 95, 100, 150 or more amino acid residues of the constant domain of a TCR alpha chain, a TCR beta chain, a TCR gamma chain or a TCR delta chain.

The TCR alpha chain, a TCR beta chain, a TCR gamma chain or a TCR delta chain described herein can be derived from various species. The TCR chain can be a murine or human TCR chain. For example, the extracellular domain can comprise a constant domain of a murine TCR alpha chain, a murine TCR beta chain, a human TCR gamma chain or a human TCR delta chain.

### Transmembrane Domain

In general, a TFP sequence can contain an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. A transmembrane domain of particular use in this present disclosure may include at least the transmembrane region(s) of e.g., the alpha, beta, gamma, delta, or zeta chain of the T cell receptor, CD28, CD3 epsilon, CD45, CD4, CD5, CD8, CD9, CD16, CD22, CD33, CD37, CD64, CD80, CD86, CD134, CD137, CD154, functional fragments thereof, and amino acid sequences thereof having at least one but not more than 20 modifications.

In some embodiments, the transmembrane domain comprises, or comprises at least 5, 6, 7, 8, 9, 10, 11, 12, 13, 14, 15, 16, 17, 18, 19, 20, 21, 22, 23, 24, 25, 26, 27, 28, 29, or 30 or more consecutive amino acid residues of the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the transmembrane domain comprises a sequence having at least about 50%, 55%, 60%, 65%, 70%, 75%, 80%, 85%, 90%, 95%, 98%, 99% or more sequence identity to a sequence encoding the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the transmembrane domain comprises a sequence encoding the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit having a truncation of at least 1, 2, 3, 4, 5, 6, 7, 8, 9, or 10 or more amino acids at the N- or C-terminus or at both the N- and C-terminus.

In some instances, the extracellular region of the TFP can be attached to the binding domain of the TFP, via a hinge, e.g., a hinge from a human protein. For example, in one embodiment, the hinge can be a human immunoglobulin (Ig) hinge, e.g., an IgG4 hinge, or a CD8a hinge.

Optionally, a short oligo- or polypeptide linker, between 2 and 10 amino acids in length may form the linkage between the binding element and the TCR extracellular domain of the TFP. In some cases, the linker may be at least about 5, 6, 7, 8, 9, 10, 11, 12, 13, 14, 15, 16, 17, 18, 19, 20, or more in length. A glycine-serine doublet provides a particularly suitable linker. For example, in one aspect, the linker comprises the amino acid sequence of GGGGSGGGGS or a sequence (GGGGS)x or (G₄S)ₙ, wherein X or n is 1, 2, 3, 4, 5, 6, 7, 8, 9, or 10 or more. In some embodiments, X or n is an integer from 1 to 10. In some embodiments, X or n is an integer from 1 to 4. In some embodiments, X or n is 2. In some embodiments, X or n is 4. In some embodiments, the linker is encoded by a nucleotide sequence of

GGTGGCGGAGGTTCTGGAGGTGGAGGTTCC.

### Cytoplasmic Domain

The cytoplasmic domain of the TFP can include an intracellular domain. In some embodiments, the intracellular domain is from CD3 gamma, CD3 delta, CD3 epsilon, TCR alpha, TCR beta, TCR gamma, or TCR delta. In some embodiments, the intracellular domain comprises a signaling domain, if the TFP contains CD3 gamma, delta or epsilon polypeptides; TCR alpha, TCR beta, TCR gamma, and TCR delta subunits generally have short (e.g., 1-19 amino acids in length) intracellular domains and are generally lacking in a signaling domain. An intracellular signaling domain can be responsible for activation of at least one of the normal effector functions of the immune cell in which the TFP has been introduced. While the intracellular domains of TCR alpha, TCR beta, TCR gamma, and TCR delta do not have signaling domains, they are able to recruit proteins having a primary intracellular signaling domain described herein, e.g., CD3 zeta, which functions as an intracellular signaling domain. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the present disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

Examples of intracellular domains for use in the TFP of the present disclosure include the cytoplasmic sequences of the T cell receptor (TCR) and co-receptors that are able to act in concert to initiate signal transduction following antigen receptor engagement, as well as any derivative or variant of these sequences and any recombinant sequence that has the same functional capability. In some embodiments, the intracellular domain comprises the intracellular domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the intracellular domain comprises, or comprises at least 1, 2, 3, 4, 5, 6, 7, 8, 9, 10, 11, 12, 13, 14, 15, 16, 17, 18, or 19 or more consecutive amino acid residues of the intracellular domain of a TCR alpha chain, a TCR beta chain, a TCR gamma chain, or a TCR delta chain. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a TCR alpha chain, a TCR beta chain, a TCR gamma chain, or a TCR delta chain. In some embodiments, the transmembrane domain comprises a sequence encoding the intracellular domain of a TCR alpha chain, a TCR beta chain, a TCR gamma chain, or a TCR delta chain having a truncation of at least 1, 2, 3, 4, 5, 6, 7, 8, 9, or 10 or more amino acids at the N- or C-terminus or at both the N- and C-terminus.

In some embodiments, the intracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 or more consecutive amino acid residues of the intracellular domain of a CD3 epsilon TCR subunit, a CD3 gamma TCR subunit, or a CD3 delta TCR subunit. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a CD3 epsilon TCR subunit, a CD3 gamma TCR subunit, or a CD3 delta TCR subunit. In some embodiments, the intracellular domain comprises a sequence encoding the intracellular domain of a CD3 epsilon TCR subunit, a CD3 gamma TCR subunit, or a CD3 delta TCR subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

A secondary and/or costimulatory signal may be needed to generate signals through the TCR for full activation of naive T cells. Thus, naïve T cell activation can be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the present disclosure include those of CD3 zeta, FcR gamma, FcR beta, CD3 gamma, CD3 delta, CD3 epsilon, CD5, CD22, CD79a, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like.

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target or a different target. In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a V_{HH}.

In another aspect, the TFP-expressing cell described herein can further express another agent, e.g., an agent which enhances the activity of a modified T cell. For example, in one embodiment, the agent can be an agent which inhibits an inhibitory molecule. Inhibitory molecules, e.g., PD1, can, in some embodiments, decrease the ability of a modified T cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al., 1996, Int. Immunol 8:765-75). Two ligands for PD1, PD-L1 and PD-L2, have been shown to downregulate T cell activation upon binding to PD1 (Freeman et al., 2000 J. Exp. Med. 192:1027-34; Latchman et al., 2001 Nat. Immunol. 2:261-8; Carter et al., 2002 Eur. J. Immunol. 32:634-43). PD-L1 is abundant in human cancers (Dong et al., 2003 J. Mol. Med. 81:281-7; Blank et al., 2005 Cancer Immunol. Immunother. 54:307-314; Konishi et al., 2004 Clin. Cancer Res. 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-autoantigen TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having a binding domain described herein, and a second cell expressing a TFP having a different anti-autoantigen binding domain, e.g., a binding domain described herein that differs from the binding domain in the TFP expressed by the first cell. As another example, the population of TFP-expressing cells can include a first cell expressing a TFP that includes a first binding domain binding domain, e.g., as described herein, and a second cell expressing a TFP that includes an antigen binding domain to a target other than the binding domain of the first cell (e.g., another autoimmune antigen).

In another aspect, the present disclosure provides a population of cells wherein at least one cell in the population expresses a TFP having a domain described herein, and a second cell expressing another agent, e.g., an agent which enhances the activity of a modified T cell. For example, in one embodiment, the agent can be an agent which inhibits an inhibitory molecule. Inhibitory molecules, e.g., can, in some embodiments, decrease the ability of a modified T cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present disclosure also includes a TFP-encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect, the anti-autoantigen TFP is encoded by a messenger RNA (mRNA). In one aspect the mRNA encoding the anti-autoantigen TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

For additional information on making and using TFP T cells, see U.S. Pat. Nos. 10,442,849, 10,358,473, 10,358,474, and 10,208,285, each of which is herein incorporated by reference.

### Nucleic Acid Constructs Encoding a TFP

The present disclosure provides nucleic acid molecules encoding one or more TFP constructs described herein. In one aspect, the nucleic acid molecule is provided as a messenger RNA transcript. In one aspect, the nucleic acid molecule is provided as a DNA construct.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

In some embodiments, the recombinant nucleic acid molecule can further comprise a sequence encoding a TCR constant domain, wherein the TCR constant domain is a TCR alpha constant domain, a TCR beta constant domain, a TCR alpha constant domain and a TCR beta constant domain, a TCR gamma constant domain, a TCR delta constant domain, or a TCR gamma constant domain and a TCR delta constant domain. The TCR subunit and the antibody can be operatively linked. The TFP can functionally incorporate into a TCR complex (e.g., an endogenous TCR complex) when expressed in a T cell.

The TCR alpha chain, a TCR beta chain, a TCR gamma chain or a TCR delta chain described herein can be derived from various species. The TCR chain can be a murine or human TCR chain. For example, the constant domain can comprise a constant domain of a murine or human TCR alpha chain, TCR beta chain, TCR gamma chain or TCR delta chain.

In some instances, the recombinant nucleic acid encodes a TFP comprising (i) a binding domain and (ii) at least a portion of a TCR extracellular domain, a TCR transmembrane domain, and a TCR intracellular domain of CD3 epsilon, CD3 gamma, or CD3 delta, and the recombinant nucleic acid further encodes the constant domain of a TCR alpha chain, a TCR beta chain, a TCR gamma chain or a TCR delta chain. In some embodiments, the recombinant nucleic acid further encodes a constant domain of a TCR alpha chain and a TCR beta chain. In some embodiments, the recombinant nucleic acid further encodes a constant domain of a TCR gamma chain and a TCR delta chain.

In some instances, the recombinant nucleic acid encodes a TFP comprising (i) a binding domain and (ii) the constant domain of a TCR alpha chain (i.e., comprising at least a portion of a TCR extracellular domain, a TCR transmembrane domain, and a TCR intracellular domain), and the recombinant nucleic acid further encodes the constant domain of a TCR beta chain. The sequence encoding the TCR beta constant domain can further encode a second binding domain that is operatively linked to the sequence encoding the TCR beta constant domain. The second binding domain can be the same or different as the binding domain of the TFP.

In some instances, the recombinant nucleic acid encodes a TFP comprising (i) a binding domain and (ii) the constant domain of a TCR beta chain (i.e., comprising at least a portion of a TCR extracellular domain, a TCR transmembrane domain, and a TCR intracellular domain), and the recombinant nucleic acid further encodes the constant domain of a TCR alpha chain. The sequence encoding the TCR alpha constant domain can further encode a second binding domain that is operatively linked to the sequence encoding the TCR alpha constant domain. The second binding domain can be the same or different as the binding domain of the TFP.

In some instances, the recombinant nucleic acid encodes a TFP comprising (i) a binding domain and (ii) the constant domain of a TCR gamma chain (i.e., comprising at least a portion of a TCR extracellular domain, a TCR transmembrane domain, and a TCR intracellular domain), and the recombinant nucleic acid further encodes the constant domain of a TCR delta chain. The sequence encoding the TCR delta constant domain can further encode a second binding domain that is operatively linked to the sequence encoding the TCR delta constant domain. The second binding domain can be the same or different as the binding domain of the TFP.

In some instances, the recombinant nucleic acid encodes a TFP comprising (i) a binding domain and (ii) the constant domain of a TCR delta chain (i.e., comprising at least a portion of a TCR extracellular domain, a TCR transmembrane domain, and a TCR intracellular domain), and the recombinant nucleic acid further encodes the constant domain of a TCR gamma chain. The sequence encoding the TCR gamma constant domain can further encode a second binding domain that is operatively linked to the sequence encoding the TCR gamma constant domain. The second binding domain can be the same or different as the binding domain of the TFP.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP of the disclosure is an adenoviral vector (A5/35). In another embodiment, the expression of nucleic acids encoding TFPs can be accomplished using of transposons such as sleeping beauty, crisper, CAS9, and zinc finger nucleases (See, June et al. 2009 Nature Reviews Immunol. 9.10: 704-716, incorporated herein by reference).

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell can be calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

The present disclosure further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian T cells. In one aspect, the mammalian T cell is a human T cell.

Disclosed herein are methods for producing in vitro or in vivo transcribed RNA encoding TFPs. In preferred embodiments, the RNA is circRNA. In some embodiments, circRNA is exogenous. In other embodiments, circRNA is endogenous. In other embodiments, circRNAs with an internal ribosomal entry site (IRES) can be translated in vitro or ex vivo.

Circular RNAs (circRNAs) are a class of single-stranded RNAs with a contiguous structure that have enhanced stability and a lack of end motifs necessary for interaction with various cellular proteins. CircRNAs are 3-5′ covalently closed RNA rings, and circRNAs do not display Cap or poly(A) tails. Since circRNAs lack the free ends necessary for exonuclease-mediated degradation, rendering them resistant to several mechanisms of RNA turnover and granting them extended lifespans as compared to their linear mRNA counterparts. For this reason, circularization may allow for the stabilization of mRNAs that generally suffer from short half-lives and may therefore improve the overall efficacy of mRNA in a variety of applications. CircRNAs are produced by the process of splicing, and circularization occurs using conventional splice sites mostly at annotated exon boundaries (Starke et al., 2015; Szabo et al., 2015). For circularization, splice sites are used in reverse: downstream splice donors are “backspliced” to upstream splice acceptors (see Jeck and Sharpless, 2014; Barrett and Salzman, 2016; Szabo and Salzman, 2016; Holdt et al., 2018 for review).

To generate circRNAs that can subsequently be transferred into cells, in vitro production of circRNAs with autocatalytic-splicing introns can be programmed. A method for generating circRNA can involve in vitro transcription (IVT) of a precursor linear RNA template with specially designed primers. Three general strategies have been reported so far for RNA circularization: chemical methods using cyanogen bromide or a similar condensing agent, enzymatic methods using RNA or DNA ligases, and ribozymatic methods using self-splicing introns. In preferred embodiments, precursor RNA was synthesized by run-off transcription and then heated in the presence of magnesium ions and GTP to promote circularization. RNA so produced can efficiently transfect different kinds of cells. In one aspect, the template includes sequences for the TFP.

The group I intron of phage T4 thymidylate synthase (td) gene can be well characterized to circularize while the exons linearly splice together (Chandry and Bel-fort, 1987; Ford and Ares, 1994; Perriman and Ares, 1998). When the td intron order is permuted flanking any exon sequence, the exon is circularized via two autocatalytic transesterification reactions (Ford and Ares, 1994; Puttaraju and Been, 1995). In preferred embodiments, the group I intron of phage T4 thymidylate synthase (td) gene is used to generate exogenous circRNA.

In some exemplary embodiments, a ribozymatic method utilizing a permuted group I catalytic intron has been used since it is more applicable to long RNA circularization and requires only the addition of GTP and Mg2⁺ as cofactors. This permuted intron-exon (PIE) splicing strategy consists of fused partial exons flanked by half-intron sequences. In vitro, these constructs undergo the double transesterification reactions characteristic of group I catalytic introns, but because the exons are fused, they are excised as covalently 5′ and 3′linked circles.

In one aspect, disclosed herein is a sequence containing a full-length encephalomyocarditis virus (such as EMCV) IRES, a gene encoding a TFP, two short regions corresponding to exon fragments (E1 and E2), and of the PIE construct between the 3′ and 5′ introns of the permuted group I catalytic intron in the thymidylate synthase (Td) gene of the T4 phage or the permuted group I catalytic intron in the pre-tRNA gene of Anabaena. In more preferred embodiments, the mentioned sequence further comprises complementary ‘homology arms’ placed at the 5′ and 3′ends of the precursor RNA with the aim of bringing the 5′ and 3′ splice sites into proximity of one another. To ensure that the major splicing product was circular, the splicing reaction can be treated with RNase R.

### Engineered Regulatory T Cells

The present disclosure provides engineered (e.g., modified) cells that can be used to function as a regulatory T cell.

Inflammation can be part of the complex biological response of body tissues to harmful stimuli, such as pathogens, damaged cells, or irritants, and can be a protective response involving immune cells, blood vessels, and molecular mediators. The function of inflammation can eliminate the initial cause of cell injury, clear out necrotic cells and tissues damaged from the original insult and the inflammatory process, and to initiate tissue repair. Inflammation can occur from infection or as a symptom of a disease, e.g., autoimmune diseases, atherosclerosis, allergies, myopathies, HIV, obesity, or an autoimmune disease.

An autoimmune disease can be a chronic condition arising from an abnormal immune response to a self-antigen. Autoimmune diseases that may be treated with the TFP T cells disclosed herein and variants thereof include but are not limited to uveitis, bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus), multiple sclerosis, neuromyelitis optica (NMO), autoimmune limbic encephalitis (LE), Hashimoto's disease, N-methyl-D-aspartate receptor (NMDAR) encephalitis, autoimmune hemolytic anemia, pemphigus vulgaris, myasthenia gravis, Graves' disease, idiopathic thrombocytopenic purpura, Goodpasture's syndrome, rheumatoid arthritis, celiac disease, pernicious anemia, vitiligo, scleroderma, psoriasis, ulcerative colitis (UC), Crohn's disease, Sjogren's syndrome, Wegener granulomatosis, polymyositis, dermatomyositis, primary biliary cirrhosis, antiphospholipid syndrome, mixed connective tissue disease, Miller Fisher syndrome, Guillain-Barre syndrome, acute motor axonal neuropathy, autoimmune hepatitis, dermatitis herpetiformis, Churg-Strauss disease, microscopic polyangiitis, IgA nephropathy, vasculitis caused by ANCA and other ANCA associated diseases, acute rheumatic fever, pernicious anemia, type 1 diabetes (T1D), reactive arthritis (Reiter syndrome), membranous nephropathy, chronic inflammatory demyelinating polyneuropathy, thrombotic thrombocytopenic purpura, hyperviscosity in monoclonal gammopathies, hemolytic uremic syndrome (atypical, due to antibody to factor H), Wilson disease, fulminant, Lambert-Eaton myasthenic syndrome, RBC alloimmunization in pregnancy, mushroom poisoning, acute disseminated encephalomyelitis, hemolytic uremic syndrome (atypical, due to complement factor mutations), autoimmune hemolytic anemia (life-threatening cold agglutinin disease), myeloma cast nephropathy, post-transfusion purpura, autoimmune hemolytic anemia (warm autoimmune hemolytic anemia), hypertriglyceridemic pancreatitis, thyroid storm, stiff person syndrome, Hemolytic uremic syndrome (typical diarrhea-associated), immune thrombocytopenia, ABO-incompatible solid organ transplantation (SOT), cryoglobulinemia, heparin-induced thrombocytopenia, thyroid storm, chronic inflammatory demyelinating polyradiculoneuropathy, focal segmental glomerulosclerosis and fulminant hepatic failure.

Regulatory T cells (Tregs) can be important in the maintenance of immune cell homeostasis as evidenced by the catastrophic consequences of genetic or physical ablation of the Treg population. Specifically, Treg cells can maintain order in the immune system by enforcing a dominant negative regulation on other immune cells. Broadly classified into natural or adaptive (e.g., induced) Tregs; natural Tregs are CD4⁺CD25⁺ T cells which develop and emigrate from the thymus to perform their key role in immune homeostasis. Adaptive Tregs can be non-regulatory CD4⁺ T cells which acquire CD25 (IL-2R alpha) expression outside of the thymus, and are typically induced by inflammation and disease processes, such as autoimmunity and autoimmune diseases.

Tregs can manifest their function through a myriad of mechanisms that include the secretion of immunosuppressive soluble factors such as IL-9, IL-10, and TGF beta, cell-contact-mediated regulation via the high affinity TCR and other costimulatory molecules such as CTLA-4, GITR, and cytolytic activity. Under the influence of TGF beta, adaptive Treg cells mature in peripheral sites, including mucosa-associated lymphoid tissue (MALT), from CD4⁺ Treg precursors, where they acquire the expression of markers typical of Tregs, including CD25, CTLA4 and GITR/AITR. Upon up-regulation of the transcription factor Foxp3, Treg cells begin their suppressive effect. This can include the secretion of cytokines such as IL-10 and TGF beta which may induce cell-cycle arrest or apoptosis in effector T cells and blocking co-stimulation and maturation of dendritic cells.

The engineered cell (e.g., an engineered T cell, or an engineered regulatory T cell) described herein can originally be a T cell isolated from a human subject. The engineered cell can comprise a recombinant nucleic acid molecule encoding a T cell receptor (TCR) fusion protein (TFP). The TFP can comprise (i) a TCR-integrating subunit comprising (1) an extracellular domain, (2) a TCR transmembrane domain, and (3) a TCR intracellular domain comprising a stimulatory domain from an intracellular signaling domain; and (ii) a binding domain. The TCR-integrating subunit and the binding domain can be operatively linked. The TFP can functionally interact with an endogenous TCR when expressed in a T cell. The binding domain can be selected from: an antigen binding domain; a T cell receptor ligand, e.g., a peptide-MHC complex; or a T cell receptor mimic, e.g., that binds the peptide-MHC complex.

The engineered cell can further comprise a gene that stimulates and/or stabilizes the formation of Tregs. The gene that stimulates and/or stabilizes the formation of Tregs can be encoded by the same recombinant nucleic acid molecule as the recombinant nucleic acid molecule encoding the TFP. The gene that stimulates and/or stabilizes the formation of Tregs can be encoded by a different recombinant nucleic acid molecule than the recombinant nucleic acid molecule encoding the TFP. The gene that stimulates and/or stabilizes the formation of Tregs can be FOXP3, HELIOS, BACH2, or pSTAT5. The engineered cell can further comprise a switch receptor.

The switch receptor can be encoded by the same recombinant nucleic acid molecule as the recombinant nucleic acid molecule encoding the TFP. The switch receptor can be encoded by a different recombinant nucleic acid molecule than the recombinant nucleic acid molecule encoding the TFP. The switch receptor can be an IL7-IL2 switch receptor, an IL7-IL10 switch receptor, or a TNF-alpha-IL2 switch receptor. The Treg can comprise more than one gene that stimulates and/or stabilizes the formation of Tregs and/or more than one switch receptor. The expression of one or more of PKC theta, STUB1, and CCAR2 in the Treg cell can be reduced or eliminated. The expression of one or more of CDK8 and CDK19 reduced, deleted, or pharmacologically inhibited to stabilized Treg formation. The Treg can be a CD4+ CD25+ FoxP3+ Treg or a CD8+ regulatory T cell.

The cells used to carry or express the recombinant nucleic acid encoding a TFP can be isolated from a subject. The cells generally can be eukaryotic cells, such as mammalian cells, and typically are human cells. In some embodiments, the cells can be derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, CD4⁺ CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naive T (TN) cells, effector T cells (TEEF), memory T cells and sub-types thereof, such as stem cell memory T (TSCMX central memory T (TCM effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive T regulatory cells, helper T cells, such as T_{H}I cells, T_{H}2 cells, T_{H}3 cells, T_{H}17 cells, T_{H}9 cells, T_{H}22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the TFP, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed, and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal., or depletion of cells of a particular type, such as those expressing a marker, refers to decreasing the number or percentage of such cells, but need not result in a complete removal of all such cells.

In some examples, multiple rounds of separation steps can be carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4⁺, CD25⁺, CD127, FOXP3⁺ and/or Helios⁺ T cells, are isolated by positive or negative selection techniques. For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation can be carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker “1”) at a relatively higher level (marker “1”^{high}) on the positively or negatively selected cells, respectively.

In some embodiments, T cells can be separated from a PBMC sample by negative selection of markers expressed on non-T cells, such as B cells, monocytes, or other white blood cells, such as CD 14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival., expansion, and/or engraftment following administration, which in some aspects is particularly robust in such sub-populations. See Terakura et al., (2012) Blood 1:72-82; Wang et al., (2012) J. Immunother. 35(9):689-701. In some embodiments, combining Tc_{M}-enriched CD8⁺ T cells and CD4⁺ T cells further enhances efficacy.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B.

In some aspects, a CD4 expression-based selection step is used to generate the CD4⁺ cell population or sub-population, such that both the positive and negative fractions from the CD4-based separation are retained and used in subsequent steps of the methods, optionally following one or more further positive or negative selection steps.

In one example, a sample of PBMCs or other white blood cell sample is subjected to selection of CD4⁺ cells, where both the negative and positive fractions are retained. The negative fraction then is subjected to negative selection based on expression o, for example, CD14 and CD45RA, and positive selection based on a marker characteristic of central memory T cells, such as CD62L or CCR7, where the positive and negative selections are carried out in either order.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁺, CD45RA⁺, CD62L⁺, CD4⁺ T cells. In some embodiments, central memory CD4⁺ cells are CD62L⁺ and CD45RO⁺.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material., such as magnetically responsive particles or microparticles, such as paramagnetic beads (e.g., such as Dynabeads or MACS beads). The magnetically responsive material., e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in U.S. Pat. Nos. 4,795,698, and 5,200,084 are other examples.

The incubation generally can be carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies or biotinylated peptides.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff el al. (2012) J. Immunother. 35(9): 651-660, Terakura et al., (2012) Blood. 1:72-82, and Wang el al. (2012) J. Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., (2010) Lab Chip 10, 1567-1573; and Godin et al., (2008) J. Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial., culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex.

In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al.) Klebanoff et al., (2012) J Immunother. 35(9): 651-660, Terakura et al., (2012) Blood. 1:72-82, and/or Wang et al., (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In one embodiment, the genetically modified Treg cells of the Treg cell population can be allogeneic Treg cells. For example, the allogeneic Treg cell can be a T cell lacking expression of a functional human leukocyte antigen (HLA), e.g., HLA class I and/or HLA class II.

In one embodiment, a Treg cell described herein (or variations thereof) can be engineered such that it does not express a functional HLA on its surface. For example, a Treg cell described herein, can be engineered such that cell surface expression HLA, e.g., HLA class 1 and/or HLA class II or non-classical HLA molecules is downregulated. In some embodiments, the Treg described herein can be engineered such that it does not express or expresses reduced levels of one or more of PKC theta, STUB1, CCAR2, CDK8 and CDK19.

Modified Treg cells that lack expression or have reduced expression of HLA, PKC theta, STUB1, CCAR2, CDK8 and CDK19 can be obtained by any suitable means, including knockout or knock down. For example, the Treg cell can include a knock down of HLA, PKC theta, STUB1, CCAR2, CDK8 or CDK19 using siRNA, shRNA, clustered regularly interspaced short palindromic repeats (CRISPR) transcription-activator like effector nuclease (TALEN), zinc finger endonuclease (ZFN), meganuclease (also known as homing endonuclease), or megaTAL (combining a TAL effector with a meganuclease cleavage domain).

In one embodiment, HLA, PKC theta, STUB1, CCAR2, CDK8 or CDK19 expression can be inhibited using siRNA or shRNA that target a nucleic acid encoding a HLA, PKC theta, STUB1, CCAR2, CDK8 or CDK19 in a T cell. Expression of siRNA and shRNAs in T cells can be achieved using any conventional expression system, e.g., such as a lentiviral expression system. Exemplary siRNA and shRNA that downregulate expression of HLA class I and/or HLA class II genes are described, e.g., in U.S. Patent Publication No. 20070036773.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or —when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a pre-determined DNA sequence-18 basepairs in length. By fusing this engineered protein domain to the Fokl nuclease, it is possible to target DNA breaks with genome-level specificity. ZFNs have been used extensively to target gene addition, removal, and substitution in a wide range of eukaryotic organisms (reviewed in Durai et al. (2005) Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fokl nuclease domain (reviewed in Mak et al. (2013), Curr Opin StructBiol. 23:93-9). In this case, however, the DNA binding domain comprises a tandem array of TAL-effector domains, each of which specifically recognizes a single DNA basepair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-TevI homing endonuclease. Unlike Fokl, I-TevI does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or an RNA duplex comprising an 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy. 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, must be efficiently delivered to the cells of interest. Delivery methods such as physical, chemical, and viral methods are also know in the art (Mali (2013). Indian J. Hum. Genet. 19: 3-8.). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

Sources of T Cells

Prior to expansion and genetic modification, a source of T cells is obtained from a subject. The T cells then can be used to introduce a recombinant nucleic acid molecule encoding a TFP described herein to make an engineered (or modified T cell). The T cell can be a regulatory T cell.

The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present invention, any number of T-cell lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the COBE® 2991 cell processor, the Baxter CytoMate®, or the Haemonetics® Cell Saver® 5) according to the manufacturer's instructions. After washing, the cells may be resuspended in a variety of biocompatible buffers, such as, for example, Ca-free, Mg-free PBS, PlasmaLyte® A, or other saline solution with or without buffer. Alternatively, the undesirable components of the apheresis sample may be removed, and the cells directly resuspended in culture media.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. In some aspects, regulatory T cells are isolated by positive selection of CD4+ cells. In some embodiments, regulatory T cells are isolated by negative selection of CD8+ T cells. In some embodiments, regulatory T cells are isolated by positive selection of CD25+ cells. In some embodiments, negative selection of CD8+T cells or positive selection of CD4+ T cells is followed by selection of CD25+ T cells. In some embodiments, regulatory T cells are isolated by negative selection of CD127+ cells. In some embodiments, negative selection of CD8+ T cells or positive selection of CD4+ T cells followed by selection of CD25+ T cells is followed by negative selection of CD127+ T cells. In some embodiments, regulatory T cells are isolated by positive selection of CD45RA+ cells. In some embodiments, negative selection of CD8+ T cells or positive selection of CD4+ T cells followed by selection of CD25+ T cells followed by negative selection of CD127+ T cells is followed by positive selection of CD45RA+ T cells. In some embodiments, CD4+ CD25+ CD45RA+ CD127^{dim/-} Treg cells are selected.

In one aspect, T cells are isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4⁺, CD25⁺, CD62Lhi, GITR⁺, and FoxP3⁺. Alternatively, in certain aspects, T regulatory cells are depleted by anti-C25 conjugated beads or other similar method of selection.

In one embodiment, a T cell population can be selected that expresses one or more of IFN-7, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan® and PlasmaLyte® A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and tacrolimus (FK506), antibodies, or other immunoablative agents such as CAMPATH, anti-CD3 antibodies, cyclophosphamide, fludarabine, cyclosporin, rapamycin, mycophenolic acid, steroids, romidepsin (formerly FR901228), and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T-cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999). In some embodiments, T cells are activated by stimulation with an anti-CD3 antibody and an anti-CD28 antibody in combination with cytokines that bind the common gamma-chain (e.g., IL-2, IL-7, IL-12, IL-15, IL-21, and others). In some embodiments, T cells are activated by stimulation with an anti-CD3 antibody and an anti-CD28 antibody in combination with 10, 20, 30, 40, 50, 60, 70, 80, 90, 100, 200, 300, 400, 500, 600, 700, 800, 900, or 100 U/mL of IL-2, IL-7, and/or IL-15. In some embodiments, the cells are activated for 24 hours. In some embodiments, after transduction, the cells are expanded in the presence of anti-CD3 antibody, anti-CD28 antibody in combination with the same cytokines. In some embodiments, cells activated in the presence of activated by stimulation with an anti-CD3 antibody and an anti-CD28 antibody in combination with cytokines that bind the common gamma-chain are expanded in the presence of the same cytokines in the absence of the anti-CD3 antibody and anti-CD28 antibody after transduction. In some embodiments, cells are expanded for 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, Treg cells are activated in the presence of an anti-CD3 antibody and an anti-CD28 antibody in combination with rapamycin. In some embodiments, Treg cells are activated in the presence of an anti-CD3 antibody, L cells, and IL-2, e.g., in Immunocult-XF T cell media. In some embodiments, the cells are subcultured every 1, 2, 3, 4, 5, or 6 days. In some embodiments, cells are expanded for 4, 5, 6, 7, 8, 9, 10, 11, 12, 13, 14, 15, 16, 17, 18, 19, 20, 21, 22, 23, 24, 25, 26, 27, 28, 29, or 30 days.

T cells that have been exposed to varied stimulation times may exhibit different characteristics. For example, typical blood or apheresed peripheral blood mononuclear cell products have a helper T-cell population (T_{H}, CD4⁺) that is greater than the cytotoxic or suppressor T-cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of TC cells. Accordingly, depending on the purpose of treatment, infusing a subject with a T cell population comprising predominately of T_{H} cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T-cell product for specific purposes.

Once a TFP is constructed, various assays can be used to evaluate the activity of the molecule, such as but not limited to, the ability to expand T cells following stimulation, sustain T-cell expansion in the absence of re-stimulation, and immunosuppressive activities in appropriate in vitro and animal models.

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by Western blotting using an antibody to a TCR chain. The same T-cell subsets are used for SDS-PAGE analysis under non-reducing conditions to permit evaluation of covalent dimer formation.

Cytotoxicity can be assessed by a standard ⁵¹Cr-release assay (see, e.g., Milone et al., Molecular Therapy 17(8): 1453-1464 (2009)). Briefly, target cells are loaded with ⁵¹Cr (as NaCrO4, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Gene Editing Technologies

In some embodiments, the modified T cells disclosed herein are engineered using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. In this way, a chimeric construct may be engineered to combine desirable characteristics of each subunit, such as conformation or signaling capabilities. See also Sander & Joung, Nat. Biotech. (2014) v32, 347-55; and June et al., 2009 Nature Reviews Immunol. 9.10: 704-716, each incorporated herein by reference. In some embodiments, one or more of the extracellular domain, the transmembrane domain, or the cytoplasmic domain of a TFP subunit are engineered to have aspects of more than one natural TCR subunit domain (i.e., are chimeric).

Recent developments of technologies to permanently alter the human genome and to introduce site-specific genome modifications in disease relevant genes lay the foundation for therapeutic applications. These technologies are now commonly known as “genome editing.

The endogenous TCR gene encoding a TCR alpha chain, a TCR beta chain, or a TCR alpha chain and a TCR beta chain can be inactivated in the modified cell (e.g., modified T cell) described herein. The inactivation can include disruption of genomic gene locus, gene silencing, inhibition or reduction of transcription, or inhibition or reduction of translation. The endogenous TCR gene can be silenced, for example, by inhibitory nucleic acids such as siRNA and shRNA. The translation of the endogenous TCR gene can be inhibited by inhibitory nucleic acids such as microRNA. In some embodiments, gene editing techniques are employed to disrupt an endogenous TCR gene. In some embodiments, mentioned endogenous TCR gene encodes a TCR alpha chain, a TCR beta chain, or a TCR alpha chain and a TCR beta chain. In some embodiments, gene editing techniques pave the way for multiplex genomic editing, which allows simultaneous disruption of multiple genomic loci in endogenous TCR gene. In some embodiments, multiplex genomic editing techniques are applied to generate gene-disrupted T cells that are deficient in the expression of endogenous TCR, and/or human leukocyte antigens (HLAs), and/or programmed cell death protein 1 (PD-1), and/or other genes.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or -when donor DNA is present-homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a pre-determined DNA sequence-18 base pairs in length. By fusing this engineered protein domain to the Fokl nuclease, it is possible to target DNA breaks with genome-level specificity. ZFNs have been used extensively to target gene addition, removal, and substitution in a wide range of eukaryotic organisms (reviewed in Durai et al. (2005), Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fokl nuclease domain (reviewed in Mak et al. (2013), Curr Opin Struct Biol. 23:93-9). In this case, however, the DNA binding domain comprises a tandem array of TAL-effector domains, each of which specifically recognizes a single DNA base pair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-TevI homing endonuclease. Unlike Fokl, I-TevI does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or an RNA duplex comprising an 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally-occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, may need to be efficiently delivered to the cells of interest. Delivery methods such as physical, chemical, and viral methods are also know in the art (Mali (2013). Indian J. Hum. Genet. 19: 3-8.). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

As an example, the endogenous TCR gene (e.g., a TRAC locus or a TRBC locus) encoding a TCR alpha chain, a TCR beta chain, or a TCR alpha chain and a TCR beta chain can be inactivated by CRISPR/Cas9 system. The gRNA used to inactivate (e.g., disrupt) the TRAC locus can comprise a sequence of SEQ ID: 58. The gRNA used to disrupt the TRBC locus can comprise a sequence of SEQ ID: 59.

(SEQ ID NO: 58)
CTCGACCAGCTTGACATCAC.
(SEQ ID NO: 59)
ACACTGGTGTGCCTGGCCAC.

### Pharmaceutical Compositions

Pharmaceutical compositions of the present disclosure may comprise a TFP-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present disclosure may be administered in a manner appropriate to the disease to be treated (or prevented). The quantity and frequency of administration can be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T-cell compositions of the present invention are administered to a patient by intradermal or subcutaneous injection. In one aspect, the T-cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the invention may be introduced, thereby creating a TFP-expressing T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded TFP T cells of the present invention. In an additional aspect, expanded cells are administered before or following surgery.

The dosage of the above treatments to be administered to a patient can vary with the precise nature of the condition being treated and the recipient of the treatment. The scaling of dosages for human administration can be performed according to art-accepted practices. The dose for alemtuzumab (CAMPATH©), for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the invention, and one or more subsequent administrations of the TFP T cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the invention are administered to the subject (e.g., human) per week, e.g., 2, 3, or 4 administrations of the TFP T cells of the invention are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the invention are administered for at least two, three, four, five, six, seven, eight or more weeks.

In one aspect, the TFP T cells are generated using lentiviral viral vectors, such as lentivirus. TFP T cells generated that way can have stable TFP expression.

In one aspect, TFP T cells transiently express TFP vectors for 4, 5, 6, 7, 8, 9, 10, 11, 12, 13, 14, 15 days after transduction. Transient expression of TFPs can be affected by RNA TFP vector delivery. In one aspect, the TFP RNA is transduced into the T-cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten- to fourteen-day break in exposure to antigen.

If a patient is at high risk of generating an anti-TFP antibody response during the course of transient TFP therapy (such as those generated by RNA transductions), TFP T-cell infusion breaks should not last more than ten to fourteen days.

In one embodiment, said composition comprises, consists or consists essentially of an isolated and/or substantially purified monospecific Treg cell population of the invention. In one embodiment, said composition has been frozen and thawed.

Another object of the invention is a pharmaceutical composition comprising, consisting or consisting essentially of at least one monospecific Treg cell population as described hereinabove and at least one pharmaceutically acceptable excipient.

The present disclosure also provides a medicament comprising, consisting or consisting essentially of at least one monospecific Treg cell population as described hereinabove.

In one embodiment, the pharmaceutical composition or medicament comprises at least one isolated and/or substantially purified monospecific Treg cell population of the invention.

In one embodiment, the pharmaceutical composition or medicament comprises a combination of monospecific Treg cell populations as described hereinabove (. e., at least two distinct populations of monospecific Treg cell populations of the invention).

Such compositions and medicaments may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The administration of the compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection.

In one embodiment, the at least one monospecific Treg cell population of the present invention are administered by i.v. injection. The compositions of the present invention are thus in one embodiment formulated for intravenous administration.

In another embodiment, the at least one monospecific Treg cell population of the present invention may be injected directly into the site of the autoimmune disease.

### Therapeutic Applications

The present disclosure also provides method for treating an autoimmune disease in a subject in need thereof, wherein the method comprises administering a therapeutically effective amount of an engineered Treg described herein.

Autoimmune diseases that may be treated by the methods described herein include, but are not limited to, uveitis, bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus), multiple sclerosis, neuromyelitis optica (NMO), autoimmune limbic encephalitis (LE), Hashimoto's disease, N-methyl-D-aspartate receptor (NMDAR) encephalitis, autoimmune hemolytic anemia, pemphigus vulgaris, myasthenia gravis, Graves' disease, idiopathic thrombocytopenic purpura, Goodpasture's syndrome, rheumatoid arthritis, celiac disease, pernicious anemia, vitiligo, scleroderma, psoriasis, ulcerative colitis (UC), Crohn's disease, Sjogren's syndrome, Wegener granulomatosis, polymyositis, dermatomyositis, primary biliary cirrhosis, antiphospholipid syndrome, mixed connective tissue disease, Miller Fisher syndrome, Guillain-Barre syndrome, acute motor axonal neuropathy, autoimmune hepatitis, dermatitis herpetiformis, Churg-Strauss disease, microscopic polyangiitis, IgA nephropathy, vasculitis caused by ANCA and other ANCA associated diseases, acute rheumatic fever, pernicious anemia, type 1 diabetes (T1D), reactive arthritis (Reiter syndrome), membranous nephropathy, chronic inflammatory demyelinating polyneuropathy, thrombotic thrombocytopenic purpura, hyperviscosity in monoclonal gammopathies, hemolytic uremic syndrome (atypical, due to antibody to factor H), Wilson disease, fulminant, Lambert-Eaton myasthenic syndrome, RBC alloimmunization in pregnancy, mushroom poisoning, acute disseminated encephalomyelitis, hemolytic uremic syndrome (atypical, due to complement factor mutations), autoimmune hemolytic anemia (life-threatening cold agglutinin disease), myeloma cast nephropathy, post-transfusion purpura, autoimmune hemolytic anemia (warm autoimmune hemolytic anemia), hypertriglyceridemic pancreatitis, thyroid storm, stiff person syndrome, Hemolytic uremic syndrome (typical diarrhea-associated), immune thrombocytopenia, ABO-incompatible solid organ transplantation (SOT), cryoglobulinemia, heparin-induced thrombocytopenia, thyroid storm, chronic inflammatory demyelinating polyradiculoneuropathy, focal segmental glomerulosclerosis and fulminant hepatic failure.

The present disclosure also relates to at least one monospecific Treg cell population (preferably in a composition, pharmaceutical composition or medicament as described hereinabove), for treating or for use in the treatment of an autoimmune disease, such as an autoantibody-mediated autoimmune disease.

In one embodiment, the cells of the monospecific Treg cell population of the disclosure express a TFP comprising the autoantigen involved in the autoimmune disease, i.e., recognizing the autoantibody involved in the autoantibody-mediated autoimmune disease to be treated. In another embodiment the TFP comprises an antibody or fragment thereof that specifically binds the autoantigen involved in the autoimmune disease. In some embodiments, the TFP comprises an MHC-peptide complex, wherein the peptide comprises the autoantigen, or a fragment thereof. In another embodiment the TFP comprises an antibody or fragment thereof that specifically binds the MHC-peptide complex.

Examples of autoantibody-mediated autoimmune diseases include but are not limited to, bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus), multiple sclerosis, neuromyelitis optica (NMO), autoimmune limbic encephalitis (LE), Hashimoto's disease, N-methyl-D-aspartate receptor (NMDAR) encephalitis, autoimmune hemolytic anemia, pemphigus vulgaris, myasthenia gravis, Graves' disease, idiopathic thrombocytopenic purpura, Goodpasture's syndrome, rheumatoid arthritis, celiac disease, pernicious anemia, vitiligo, scleroderma, psoriasis, ulcerative colitis (UC), Crohn's disease, Sjogren's syndrome, Wegener granulomatosis, polymyositis, dermatomyositis, primary biliary cirrhosis, antiphospholipid syndrome, mixed connective tissue disease, Miller Fisher syndrome, Guillain-Barre syndrome, acute motor axonal neuropathy, autoimmune hepatitis, dermatitis herpetiformis, Churg-Strauss disease, microscopic polyangiitis, IgA nephropathy, vasculitis caused by ANCA and other ANCA associated diseases, acute rheumatic fever, pernicious anemia, type 1 diabetes (TID), reactive arthritis (Reiter syndrome), membranous nephropathy, chronic inflammatory demyelinating polyneuropathy, thrombotic thrombocytopenic purpura, hyperviscosity in monoclonal gammopathies, hemolytic uremic syndrome (atypical., due to antibody to factor H), Wilson disease, fulminant, Lambert-Eaton myasthenic syndrome, RBC alloimmunization in pregnancy, mushroom poisoning, acute disseminated encephalomyelitis, hemolytic uremic syndrome (atypical., due to complement factor mutations), autoimmune hemolytic anemia (life-threatening cold agglutinin disease), myeloma cast nephropathy, posttransfusion purpura, autoimmune hemolytic anemia (warm autoimmune hemolytic anemia), hypertriglyceridemic pancreatitis, thyroid storm, stiff person syndrome, Hemolytic uremic syndrome (typical diarrhea-associated), immune thrombocytopenia, ABO-incompatible solid organ transplantation (SOT), cryoglobulinemia, heparin-induced thrombocytopenia, thyroid storm, chronic inflammatory demyelinating polyradiculoneuropathy, focal segmental glomerulosclerosis and fulminant hepatic failure. Preferably, said autoantibody-mediated autoimmune disease is selected from the group comprising bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus) and multiple sclerosis. Examples of autoantibody-mediated autoimmune diseases include but are not limited to, uveitis, bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus), multiple sclerosis, neuromyelitis optica (NMO), autoimmune limbic encephalitis (LE), Hashimoto's disease, N-methyl-D-aspartate receptor (NMDAR) encephalitis, autoimmune hemolytic anemia, pemphigus vulgaris, myasthenia gravis, Graves' disease, idiopathic thrombocytopenic purpura, Goodpasture's syndrome, rheumatoid arthritis, celiac disease, pernicious anemia, vitiligo, scleroderma, psoriasis, ulcerative colitis (UC), Crohn's disease, Sjogren's syndrome, Wegener granulomatosis, polymyositis, dermatomyositis, primary biliary cirrhosis, antiphospholipid syndrome, mixed connective tissue disease, Miller Fisher syndrome, Guillain-Barre syndrome, acute motor axonal neuropathy, autoimmune hepatitis, dermatitis herpetiformis, Churg-Strauss disease, microscopic polyangiitis, IgA nephropathy, vasculitis caused by ANCA and other ANCA associated diseases, acute rheumatic fever, pernicious anemia, type 1 diabetes (TID), reactive arthritis (Reiter syndrome), membranous nephropathy, chronic inflammatory demyelinating polyneuropathy, thrombotic thrombocytopenic purpura, hyperviscosity in monoclonal gammopathies, hemolytic uremic syndrome (atypical., due to antibody to factor H), Wilson disease, fulminant, Lambert-Eaton myasthenic syndrome, RBC alloimmunization in pregnancy, mushroom poisoning, acute disseminated encephalomyelitis, hemolytic uremic syndrome (atypical., due to complement factor mutations), autoimmune hemolytic anemia (life-threatening cold agglutinin disease), myeloma cast nephropathy, post-transfusion purpura, autoimmune hemolytic anemia (warm autoimmune hemolytic anemia), hypertriglyceridemic pancreatitis, thyroid storm, stiff person syndrome, Hemolytic uremic syndrome (typical diarrhea-associated), immune thrombocytopenia, ABO-incompatible solid organ transplantation (SOT), cryoglobulinemia, heparin-induced thrombocytopenia, thyroid storm, chronic inflammatory demyelinating polyradiculoneuropathy, focal segmental glomerulosclerosis and fulminant hepatic failure. Preferably, said autoantibody-mediated autoimmune disease is selected from the group comprising bullous pemphigoid, lupus (including systemic lupus erythematosus (SLE), lupus nephritis (LN), discoid lupus, lupus erythematosus profundus, Chilbrain lupus erythematosus, tumidus lupus erythematosus, severe systemic lupus erythematosus, acute cutaneous lupus, chronic cutaneous lupus) and multiple sclerosis.

In one embodiment, the autoantibody-mediated autoimmune disease may be treated by apheresis. Examples of diseases that may be treated by apheresis include but are not limited to, systemic lupus erythematosus (severe, nephritis), multiple sclerosis, Guillain-Barre syndrome, Myasthenia gravis, chronic inflammatory demyelinating polyneuropathy, thrombotic thrombocytopenic purpura, hyperviscosity in monoclonal gammopathies, goodpasture syndrome, hemolytic uremic syndrome (atypical., due to antibody to factor H), Wilson disease, fulminant, Lambert-Eaton myasthenic syndrome, RBC alloimmunization in pregnancy, mushroom poisoning, acute disseminated encephalomyelitis, hemolytic uremic syndrome (atypical., due to complement factor mutations), autoimmune hemolytic anemia (life-threatening cold agglutinin disease), myeloma cast nephropathy, post-transfusion purpura, autoimmune hemolytic anemia (warm autoimmune hemolytic anemia), hypertriglyceridemic pancreatitis, thyroid storm, stiff person syndrome, Hemolytic uremic syndrome (typical diarrhea-associated), and immune thrombocytopenia.

In one embodiment, the autoantibody-mediated autoimmune disease may be treated by plasmapheresis. Examples of diseases that may be treated by plasmapheresis include but are not limited to, systemic lupus erythematosus, ABO-incompatible solid organ transplantation (SOT), thrombotic thrombocytopenic purpura, cryoglobulinemia, heparin-induced thrombocytopenia, thyroid storm, chronic inflammatory demyelinating polyradiculoneuropathy, ANCA associated diseases, focal segmental glomerulosclerosis, fulminant hepatic failure, myasthenia gravis, Goodpasture's syndrome, Guillain-Barre Syndrome, autoimmune hemolytic anemia, IgA nephropathy, hemolytic uremic syndrome.

In one embodiment, the autoantibody-mediated autoimmune disease may be treated by rituximab. Examples of autoantibody-mediated autoimmune diseases that may be treated by rituximab include, but are not limited to, lupus nephritis, systemic lupus erythematosus, rheumatoid arthritis, Wegener granulomatosis, microscopic polyangiitis, immune thrombocytopenic purpura (ITP), pemphigus vulgaris, sicca syndrome (Sjogren), glomerulonephritis, myasthenia gravis, liver transplant rejection, idiopathic thrombocytopenic purpura (immune thrombocytopenic purpura), kidney transplant rejection, hemophilia A, neuromyelitis optica (Devic's syndrome), pemphigus vulgaris, and thrombotic thrombocytopenic purpura. In one embodiment, the autoimmune disease is neuromyelitic optica (NMO), and the TFP comprises an autoantigen (or a variant or fragment thereof) associated with NMO. Examples of autoantigens associated with neuromyelitis optica (NMO) include, but are not limited to, aquaporin-4 water channel (AQP4).

In one embodiment, the autoimmune disease is LE (lupus erythematosus), and the TFP comprises an autoantigen associated with LE. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with LE include, but are not limited to, Hu, Ma2, collapsin response-mediator protein 5 (CRMP5), voltage-gated potassium channel (VGKC), N-methyl-d-aspartate receptor (NMDAR), and a-amino-3-hydroxy-5-methyl-4-isoxazoleproprionic acid (AMPAR). In one embodiment, the autoimmune disease is SLE (systemic lupus erythematosus)/LN (lupus nephritis), and the TFP comprises an autoantigen (or a variant or fragment thereof) associated with SLE/LN. Examples of autoantigens associated with SLE/LN include, but are not limited to, DNA, histone, ribosomes, and RNP.

In one embodiment, the autoimmune disease is acute cutaneous lupus erythematosus (ACLE), and the TFP comprises an autoantigen (or a variant or fragment thereof) associated with acute cutaneous lupus erythematosus. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with acute cutaneous lupus erythematosus include, but are not limited to, DNA, and RNP.

In one embodiment, the autoimmune disease is chronic cutaneous lupus erythematosus, and the TFP comprises an autoantigen (or a variant or fragment thereof) associated with chronic cutaneous lupus erythematosus. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with chronic cutaneous lupus erythematosus include, but are not limited to, RNP.

In one embodiment, the autoimmune disease is discoid lupus erythematosus/lupus erythematosus profundus/chilblain lupus erythematosus/tumidus lupus erythematosus nephropathy, and the TFP comprises an autoantigen (or a variant or fragment thereof) associated with discoid lupus erythematosus/lupus erythematosus profundus/chilblain lupus erythematosus/tumidus lupus erythematosus nephropathy. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with discoid lupus erythematosus/lupus erythematosus profundus/chilblain lupus erythematosus/tumidus lupus erythematosus nephropathy include, but are not limited to, ANA.

In one embodiment, the autoimmune disease is Hashimoto's disease, and the TFP comprises an autoantigen associated with Hashimoto's disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Hashimoto's disease include, but are not limited to, thyroid peroxidase, and thyroglobulin.

In one embodiment, the autoimmune disease is NMDAR encephalitis, and the TFP comprises an autoantigen (or a variant or fragment thereof) associated with NMDAR encephalitis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with NMDAR encephalitis include, but are not limited to, anti-N-methyl-D-aspartate receptor (NR1 subunit).

In one embodiment, the autoimmune disease is autoimmune hemolytic anemia, and the TFP comprises an autoantigen (or a variant or fragment thereof) associated with autoimmune hemolytic anemia. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with autoimmune hemolytic anemia include, but are not limited to, Rh blood group antigens, and I antigen.

In one embodiment, the autoimmune disease is pemphigus vulgaris, and the TFP comprises an autoantigen (or a variant or fragment thereof) associated with pemphigus vulgaris. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with pemphigus vulgaris include, but are not limited to, Dsgl/3.

In one embodiment, the autoimmune disease is bullous pemphigoid, and the TFP comprises an autoantigen (or a variant or fragment thereof) associated with bullous pemphigoid. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with bullous pemphigoid include, but are not limited to, BP 180, and BP230.

In one embodiment, the autoimmune disease is Myasthenia Gravis, and the TFP comprises an autoantigen (or a variant or fragment thereof) associated with Myasthenia Gravis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with myasthenia gravis include, but are not limited to, acetylcholine nicotinic postsynaptic receptors.

In one embodiment, the autoimmune disease is Graves' disease, and the TFP comprises an autoantigen (or a variant or fragment thereof) associated with Graves' disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Graves' disease include but are not limited to, thyrotropin receptors.

In one embodiment, the autoimmune disease is idiopathic thrombocytopenic purpura (ITP), and the TFP comprises an autoantigen (or a variant or fragment thereof) associated with idiopathic thrombocytopenic purpura (ITP). In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with idiopathic thrombocytopenic purpura include, but are not limited to, Platelet integrin, and GpIIb:IIIa.

In one embodiment, the autoimmune disease is Goodpasture's syndrome, and the TFP comprises an autoantigen (or a variant or fragment thereof) associated with Goodpasture's syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Goodpasture's syndrome include, but are not limited to, Collagen alpha-3(IV) chain.

In one embodiment, the autoimmune disease is rheumatoid arthritis, and the TFP comprises an autoantigen (or a variant or fragment thereof) associated with rheumatoid arthritis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with rheumatoid arthritis include, but are not limited to, Rheumatoid factor, and calpastatin.

In one embodiment, the autoimmune disease is juvenile idiopathic arthritis, and the TFP comprises an autoantigen (or a variant or fragment thereof) associated with juvenile idiopathic arthritis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with juvenile idiopathic arthritis include, but are not limited to, RF, citrullinated proteins.

In one embodiment, the autoimmune disease is multiple sclerosis, and the TFP comprises an autoantigen (or a variant or fragment thereof) associated with multiple sclerosis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with multiple sclerosis include, but are not limited to, Myelin basic protein (MBP), Myelin oligodendrocyte glycoprotein (MOG) peptides, and alpha-beta-crystallin.

In one embodiment, the autoimmune disease is celiac disease, and the TFP comprises an autoantigen (or a variant or fragment thereof) associated with celiac disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with celiac disease include, but are not limited to, tissue transglutaminase (TG2).

In one embodiment, the autoimmune disease is pernicious anemia, and the TFP comprises an autoantigen (or a variant or fragment thereof) associated with pernicious anemia. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with pernicious anemia include, but are not limited to, intrinsic factor of gastric parietal cells.

In one embodiment, the autoimmune disease is vitiligo, and the TFP comprises an autoantigen (or a variant or fragment thereof) associated with vitiligo. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with vitiligo include, but are not limited to, 65-kDa antigen.

In one embodiment, the autoimmune disease is Behcet's disease, and the TFP comprises an autoantigen (or a variant or fragment thereof) associated with Behcet's disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Behcet's disease include, but are not limited to, phosphatidylserine, ribosomal phosphoproteins, and anti-neutrophil cytoplasmic antibody.

In one embodiment, the autoimmune disease is scleroderma, and the TFP comprises an autoantigen (or a variant or fragment thereof) associated with scleroderma. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with scleroderma include, but are not limited to, Scl-70, U1-RNP.

In one embodiment, the autoimmune disease is psoriasis, and the TFP comprises an autoantigen (or a variant or fragment thereof) associated with psoriasis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with psoriasis include, but are not limited to, calpastatin.

In one embodiment, the autoimmune disease is ulcerative colitis (UC) and Crohn's disease, and the TFP comprises an autoantigen (or a variant or fragment thereof) associated with UC and Crohn's disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with UC and Crohn's disease include, but are not limited to, ANA.

In one embodiment, the autoimmune disease is Sjogren's syndrome, and the TFP comprises an autoantigen (or a variant or fragment thereof) associated with Sjogren's syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Sjogren's syndrome include, but are not limited to, SSA and anti-SSB.

In one embodiment, the autoimmune disease is Wegener's granulomatosis, and the TFP comprises an autoantigen (or a variant or fragment thereof) associated with Wegener's granulomatosis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Wegener's granulomatosis include, but are not limited to, ANA, and ANCA.

In one embodiment, the autoimmune disease is polymyositis or dermatomyositis, and the TFP comprises an autoantigen (or a variant or fragment thereof) associated with polymyositis or dermatomyositis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with polymyositis or dermatomyositis include, but are not limited to, Jo-1.

In one embodiment, the autoimmune disease is primary biliary cirrhosis, and the TFP comprises an autoantigen (or a variant or fragment thereof) associated with primary biliary cirrhosis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with primary biliary cirrhosis include, but are not limited to, anti-mitochondrial antibodies, gp210, p62, sp 100.

In one embodiment, the autoimmune disease is antiphospholipid syndrome (APS), and the TFP comprises an autoantigen (or a variant or fragment thereof) associated with antiphospholipid syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with antiphospholipid syndrome include, but are not limited to, anti-phospholipid antibodies.

In one embodiment, the autoimmune disease is mixed connective tissue disease (MCTD), and the TFP comprises an autoantigen (or a variant or fragment thereof) associated with mixed connective tissue disease. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with mixed connective tissue disease include, but are not limited to, Ul-RNP, U1-70 kd snRNP.

In one embodiment, the autoimmune disease is Miller Fisher syndrome, and the TFP comprises an autoantigen (or a variant or fragment thereof) associated with Miller Fisher syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Miller Fisher syndrome include, but are not limited to, GQlb ganglioside.

In one embodiment, the autoimmune disease is Guillain-Barre syndrome, and the TFP comprises an autoantigen (or a variant or fragment thereof) associated with Guillain-Barre syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Guillain-Barre syndrome include, but are not limited to, GM1, asialo GM1, and GDlb.

In one embodiment, the autoimmune disease is acute motor axonal neuropathy, and the TFP comprises an autoantigen (or a variant or fragment thereof) associated with acute motor axonal neuropathy. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with acute motor axonal neuropathy include, but are not limited to, GM1.

In one embodiment, the autoimmune disease is autoimmune hepatitis, and the TFP comprises an autoantigen (or a variant or fragment thereof) associated with autoimmune hepatitis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with autoimmune hepatitis include, but are not limited to, antinuclear antibodies (ANA) and anti-smooth muscle antibodies (ASMA), anti-liver-kidney microsome-1 antibodies (ALKM-1) and anti-liver cytosol antibody-1 (ALC-1).

In one embodiment, the autoimmune disease is dermatitis herpetiformis, and the TFP comprises an autoantigen (or a variant or fragment thereof) associated with dermatitis herpetiformis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with dermatitis herpetiformis include, but are not limited to, IgA anti-endomysial antibodies.

In one embodiment, the autoimmune disease is Churg-Strauss syndrome, and the TFP comprises an autoantigen (or a variant or fragment thereof) associated with Churg-Strauss syndrome. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with Churg-Strauss syndrome include, but are not limited to, anti-neutrophil cytoplasm antibodies (ANCAs).

In one embodiment, the autoimmune disease is microscopic polyangiitis, and the TFP comprises an autoantigen (or a variant or fragment thereof) associated with microscopic polyangiitis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with microscopic polyangiitis include, but are not limited to, ANCAs.

In one embodiment, the autoimmune disease is ANCA vasculitis, and the TFP comprises an autoantigen (or a variant or fragment thereof) associated with ANCA vasculitis. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with ANCA vasculitis include, but are not limited to, neutrophil granule proteins.

In one embodiment, the autoimmune disease is acute rheumatic fever, and the TFP comprises an autoantigen (or a variant or fragment thereof) associated with acute rheumatic fever. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with acute rheumatic fever include, but are not limited to, streptococcal cell wall antigen.

In one embodiment, the autoimmune disease is type 1 Diabetes (TID), and the TFP comprises an autoantigen (or a variant or fragment thereof) associated with TID. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with TID include, but are not limited to, insulin (IAA), glutamic acid decarboxylase (GAA or GAD) and protein tyrosine phosphatase (IA2 or ICA512).

In one embodiment, the autoimmune disease is membranous nephropathy, and the TFP comprises an autoantigen (or a variant or fragment thereof) associated with membranous nephropathy. In some embodiments, the TFP comprises a MHC-peptide complex in which the peptide comprises the autoantigen or fragment thereof, or an antibody or fragment thereof that specifically binds the autoantigen or MHC-peptide complex. Examples of autoantigens associated with membranous nephropathy include, but are not limited to, PLA2R1 and THSD7A1.

In one embodiment, the subject (e.g., human) receives an initial administration of the monospecific Treg cell population of the invention, and one or more subsequent administrations, wherein the one or more subsequent administrations are administered less than 15 days, e.g., 14, 13, 12, 11, 10, 9, 8, 7, 6, 5, 4, 3, or 2 days after the previous administration.

In one embodiment, the amount of Treg cells of the at least one monospecific Treg cell population of the invention administered to the subject ranges from about 10² to about 10⁹, from about 10³ to about 10⁸, from about 10⁴ to about 10⁷, or from about 10⁵ to about 10⁶ cells.

In another embodiment, the amount of Treg cells of the at least one monospecific Treg cell population of the invention administrated to the subject ranges from about 10⁶ to about 10⁹, from about 10⁶ to 10⁷, from about 10⁶ to 10⁸, from about 10⁷ to 10⁹, from about 10⁷ to 10⁸, from about 10⁸ to 10⁹. In another embodiment the amount of Treg cells of the at least one monospecific Treg cell population of the invention administrated to the subject is about 10⁶, about 10⁷, about 10⁸, or is about 10⁹.

In one embodiment, the amount of Treg cells of the at least one monospecific Treg cell population of the invention administered to the subject ranges from about 10⁴ to 10⁹ cells/kg body weight or 10⁵ to 10⁸ cells/kg body weight, including all integer values within those ranges.

In one embodiment, more than one administration of the at least one monospecific Treg cell population of the invention are administered to the subject (e.g., human) per week, e.g., 2, 3, or 4 administrations of the genetically modified CR Treg cells of the invention are administered per week.

Another object of the present disclosure is an article of manufacture containing materials useful for the treatment of an autoimmune disease.

The article of manufacture may comprise a container and a label or package insert on or associated with the container. Suitable containers include, for example, bottles, vials, syringes, pouch, etc. The containers may be formed from a variety of materials such as glass or plastic. The container can hold a composition which can be effective for treating the autoimmune disease, such as an autoantibody-mediated autoimmune disease, and may have a sterile access port (for example the container may be an intravenous solution bag or a vial having a stopper pierceable by a hypodermic injection needle). At least one active agent in the composition can be a monospecific Treg cell population of the disclosure.

The label or package insert may indicate that the composition is used for treating an autoimmune disease. The article of manufacture, label or package insert may further comprise instructional material for administering the monospecific Treg cell population of the invention to the patient. Additionally, the article of manufacture may further comprise a second container comprising a pharmaceutically acceptable buffer, such as, for example, bacteriostatic water for injection (BWFI), phosphate-buffered saline, Ringer's solution and dextrose solution. It may further include other materials desirable from a commercial and user standpoint, including other buffers, diluents, filters, needles, and syringes.

The present disclosure also provides a kit comprising at least one monospecific Treg cell population of the invention. The kit can be any manufacture (e.g., a package or a container) comprising at least one monospecific Treg cell population of the present disclosure. The kit may be promoted, distributed, or sold as a unit for performing the methods of the present invention. Furthermore, any or all of the kit reagents may be provided within containers that protect them from the external environment, such as in sealed containers.

The kits may also contain a package insert describing the kit and methods for its use. Kits can also be provided that are useful for various purposes (e.g., for treating an autoimmune disease). Kits can be provided which contain the monospecific Treg cell population of the invention. As with the article of manufacture, the kit may comprise a container and a label or package insert on or associated with the container. The container holds a composition comprising at least one monospecific Treg cell population of the invention. Additional containers may be included that contain, e.g., diluents and buffers. The label or package insert may provide a description of the composition as well as instructions for the intended use.

### Methods of Treatment

Disclosed herein are methods of treating autoimmune diseases in a subject in need thereof, the method comprising administering to the subject a therapeutically effective amount of the pharmaceutical compositions and formulations disclosed herein. Further disclosed herein, in some embodiments, are methods of treating autoimmune diseases in a subject in need thereof, the method comprising administering to the subject a pharmaceutical composition comprising (a) a modified human immune cell produced according to the methods disclosed herein; and (b) a pharmaceutically acceptable carrier. Further disclosed herein, in some embodiments, are methods of treating autoimmune diseases in a subject in need thereof, the method comprising administering to the subject a pharmaceutical composition comprising (a) a delivery device (e.g., a liposome) containing a payload comprising one of the circular RNA molecules or vectors disclosed herein; and (b) a pharmaceutically acceptable carrier.

In some instances, the modified human immune cell is an allogeneic T cell. In some embodiments, the modified human immune cell is an autologous T cell. In some embodiments, the modified human immune cell is a lymphoblast. In some instances, less cytokines are released in the subject compared a subject administered an effective amount of an unmodified control T cell. In some instances, less cytokines are released in the subject compared a subject administered an effective amount of a modified human immune cell comprising the recombinant nucleic acid disclosed herein, or the vector disclosed herein.

In some instances, the method comprises administering the pharmaceutical formulation in combination with an agent that increases the efficacy of the pharmaceutical formulation. In some instances, the method comprises administering the pharmaceutical formulation in combination with an agent that ameliorates one or more side effects associated with the pharmaceutical composition.

In one aspect, the modified human immune cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP or TCR or CAR and a TCR alpha and/or beta constant domain to the cells or iii) cryopreservation of the cells.

Cells can be isolated from a mammal (e.g., a human) and genetically modified (i.e., transduced or transfected in vitro) with a vector disclosed herein. The modified human immune cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art; therefore, the present disclosure is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised.

The modified human immune cells of the present disclosure may be administered either alone, or as a pharmaceutical composition in combination with diluents and/or with other components such as IL-2 or other cytokines or cell populations.

### Combination Therapies

A modified human immune cell (e.g., a T regulatory cell or a Treg cell) described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a modified human immune cell. Also provided are T cells that express multiple TFPs, which bind to the same or different target antigens, or same or different epitopes on the same target antigen. Also provided are populations of T cells in which a first subset of T cells expresses a first TFP and a TCR alpha and/or beta constant domain and a second subset of T cells express a second TFP and a TCR alpha and/or beta constant domain.

A modified human immune cell described herein and the at least one additional therapeutic agent can be administered simultaneously, in the same or in separate compositions, or sequentially. For sequential administration, the modified human immune cell described herein can be administered first, and the additional agent can be administered second, or the order of administration can be reversed.

In further aspects, a modified human immune cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of a modified human immune cell. Side effects associated with the administration of a modified human immune cell include but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods disclosed herein can comprise administering a modified human immune cell described herein to a subject and further administering an agent to manage elevated levels of a soluble factor resulting from treatment with a modified human immune cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is etanercept. An example of an IL-6 inhibitor is tocilizumab.

In one embodiment, the subject can be administered an agent which enhances the activity of a modified human immune cell. For example, in one embodiment, the agent can be an agent which inhibits an inhibitory molecule. Inhibitory molecules, e.g., Programmed Death 1 (PD1), can, in some embodiments, decrease the ability of a modified human immune cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a modified human immune cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a modified human immune cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy*; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of a modified human immune cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-autoantigen TFP.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention and are not to be construed as limiting in any way the remainder of the disclosure.

### Example 1: TFP Constructs

Anti-HLA-A2 TFP and MH1-TFP constructs were engineered by cloning an anti-HLA-A2 scFv (3PF12) or anti-MLSN (MHle VHH) DNA fragment linked to CD3 epsilon DNA fragment using a (G₄S)₃ linker sequence (GGGGGTGGAGGCTCTGGAGGGGGCGGTAGTGGTGGCGGAGGAAGC (SEQ ID NO: 1)) (SL): AAAGGGGSGGGGSGGGGSLE (SEQ ID NO: 56), or (LL): AAAIEVMYPPPYLGGGGSGGGGSGGGGSLE (SEQ ID NO: 57) into a lentiviral vector (pLRPO, pLRPC, pLKaUS or pLCUS). In some instances, the vector further comprises a sequence encoding a FoxP3 gene downstream of the TFP sequence separated from the TFP sequence by a cleavable 2A (P2A or T2A) peptide. In some instances, the vector further encodes a truncated EGFR safety switch downstream of the FoxP3 gene separated from the FoxP3 sequence by a cleavable 2A (P2A or T2A) peptide Various other vectors may be used to generate fusion protein constructs. Any TFP, e.g., any TFP described herein, can be used.

The V_{H} domain of the scFv is as follows:

(SEQ ID NO: 2)
ACAGGTGCAGCTGGTGCAGTCTGGGGGAGGCGTGGTCCAGCCTGGGGGGT
CCCTGAGAGTCTCCTGTGCAGCGTCTGGGGTCACCCTCAGTGATTATGGC
ATGCATTGGGTCCGCCAGGCTCCAGGCAAGGGGCTGGAGTGGATGGCTTT
TATACGGAATGATGGAAGTGATAAATATTATGCAGACTCCGTGAAGGGCC
GATTCACCATCTCCAGAGACAACTCCAAGAAAACAGTGTCTCTGCAAATG
AGCAGTCTCAGAGCTGAAGACACGGCTGTGTATTACTGTGCGAAAAATGG
CGAATCTGGGCCTTTGGACTACTGGTACTTCGATCTCTGGGGCCGTGGCA
CCCTGGTCACCGTGTCGAGT

The VL domain of the scFv is as follows:

(SEQ ID NO: 3)
GATGTTGTGATGACTCAGTCTCCATCCTCCCTGTCTGCATCTGTAGGAGA
CAGAGTCACCATCACTTGCCAGGCGAGTCAGGACATTAGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAGGATTTTG
CAACTTATTACTGCCAACAATATAGTAGTTTTCCGCTCACTTTCGGCGGA
GGGACCAAAGTGGATATCAAACGT

Various linker configurations can be utilized. TCR alpha and TCR beta chains can be used for generation of TFPs either as full-length polypeptides or only their constant domains. Any variable sequence of TCR alpha and TCR beta chains is allowed for making TFPs.

### Source of TCR Subunits

Subunits of the human T Cell Receptor (TCR) complex all contain an extracellular domain, a transmembrane domain, and an intracellular domain. A human TCR complex contains the CD3-epsilon polypeptide, the CD3-gamma polypeptide, the CD3-delta polypeptide, the CD3-zeta polypeptide, the TCR alpha chain polypeptide and the TCR beta chain polypeptide. The human CD3-epsilon polypeptide canonical sequence is Uniprot Accession No. P07766. The human CD3-gamma polypeptide canonical sequence is Uniprot Accession No. P09693. The human CD3-delta polypeptide canonical sequence is Uniprot Accession No. P043234. The human CD3-zeta polypeptide canonical sequence is Uniprot Accession No. P20963. The human TCR alpha chain canonical sequence is Uniprot Accession No. Q6ISU1. The murine TCR alpha chain canonical sequence is Uniprot Accession No. A0A075B662. The human TCR beta chain C region canonical sequence is Uniprot Accession No. P01850, a human TCR beta chain V region sequence is P04435. The murine TCR beta chain constant region canonical sequence is Uniprot Accession No. P01852.

| SEQ | | |
|---|---|---|
| ID | | |
| NO. | Name | Sequence |
| 4 | Short Linker 1 | GGGGSGGGGSGGGGSLE |
| 5 | Short Linker 2 | AAAGGGGSGGGGSGGGGSLE |
| 6 | Long Linker | AAAIEVMYPPPYLGGGGSGGGGSGGGGSLE |
| 7 | human CD3-ε | MQSGTHWRVLGLCLLSVGVWGQDGNEEMGGITQTPYKVSISG |
| | | TTVILTCPQYPGSEILWQHNDKNIGGDEDDKNIGSDEDHLSLKE |
| | | FSELEQSGYYVCYPRGSKPEDANFYLYLRARVCENCMEMDVM |
| | | SVATIVIVDICITGGLLLLVYYWSKNRKAKAKPVTRGAGAGGR |
| | | QRGQNKERPPPVPNPDYEPIRKGQRDLYSGLNQRRI |
| 8 | mature human | DGNEEMGGITQTPYKVSISGTTVILTCPQYPGSEILWQHNDKNIG |
| | CD3-epsilon | GDEDDKNIGSDEDHLSLKEFSELEQSGYYVCYPRGSKPEDANFY |
| | | LYLRARVCENCMEMDVMSVATIVIVDICITGGLLLLVYYWSKN |
| | | RKAKAKPVTRGAGAGGRQRGQNKERPPPVPNPDYEPIRKGQRD |
| | | LYSGLNQRRI |
| 9 | signal peptide | MQSGTHWRVLGLCLLSVGVWGQ |
| | of human | |
| | CD3ε | |
| 10 | extracellular | DGNEEMGGITQTPYKVSISGTTVILTCPQYPGSEILWQHNDKNIG |
| | domain of | GDEDDKNIGSDEDHLSLKEFSELEQSGYYVCYPRGSKPEDANFY |
| | human CD3ε | LYLRARVCENCMEMD |
| 11 | transmembrane | VMSVATIVIVDICITGGLLLLVYYWS |
| | domain of | |
| | human CD3ε | |
| 12 | intracellular | KNRKAKAKPVTRGAGAGGRQRGQNKERPPPVPNPDYEPIRKG |
| | domain of | QRDLYSGLNQRRI |
| | human CD3ε | |
| 13 | human CD3-γ | MEQGKGLAVLILAIILLQGTLAQSIKGNHLVKVYDYQEDGSVLL |
| | | TCDAEAKNITWFKDGKMIGFLTEDKKKWNLGSNAKDPRGMYQ |
| | | CKGSQNKSKPLQVYYRMCQNCIELNAATISGFLFAEIVSIFVLAV |
| | | GVYFIAGQDGVRQSRASDKQTLLPNDQLYQPLKDREDDQYSHL |
| | | QGNQLRRN |
| 14 | mature human | QSIKGNHLVKVYDYQEDGSVLLTCDAEAKNITWFKDGKMIGFL |
| | CD3-gamma | TEDKKKWNLGSNAKDPRGMYQCKGSQNKSKPLQVYYRMCQN |
| | | CIELNAATISGFLFAEIVSIFVLAVGVYFIAGQDGVRQSRASDKQ |
| | | TLLPNDQLYQPLKDREDDQYSHLQGNQLRRN |
| 15 | signal peptide | MEQGKGLAVLILAIILLQGTLA |
| | of human | |
| | CD3γ | |
| 16 | extracellular | QSIKGNHLVKVYDYQEDGSVLLTCDAEAKNITWFKDGKMIGFL |
| | domain of | TEDKKKWNLGSNAKDPRGMYQCKGSQNKSKPLQVYYRMCQN |
| | human CD3γ | CIELNAATIS |
| 17 | transmembrane | GFLFAEIVSIFVLAVGVYFIA |
| | domain of | |
| | human CD3 γ | |
| 18 | intracellular | GQDGVRQSRASDKQTLLPNDQLYQPLKDREDDQYSHLQGNQL |
| | domain of | RRN |
| | human CD3γ | |
| 19 | human CD3-δ | MEHSTFLSGLVLATLLSQVSPFKIPIEELEDRVFVNCNTSITWVE |
| | | GTVGTLLSDITRLDLGKRILDPRGIYRCNGTDIYKDKESTVQVH |
| | | YRMCQSCVELDPATVAGIIVTDVIATLLLALGVFCFAGHETGRL |
| | | SGAADTQALLRNDQVYQPLRDRDDAQYSHLGGNWARNKS |
| 20 | mature human | FKIPIEELEDRVFVNCNTSITWVEGTVGTLLSDITRLDLGKRILDP |
| | CD3-delta | RGIYRCNGTDIYKDKESTVQVHYRMCQSCVELDPATVAGIIVTD |
| | | VIATLLLALGVFCFAGHETGRLSGAADTQALLRNDQVYQPLRD |
| | | RDDAQYSHLGGNWARNKS |
| 21 | signal peptide | MEHSTFLSGLVLATLLSQVSP |
| | of human | |
| | CD3δ | |
| 22 | extracellular | FKIPIEELEDRVFVNCNTSITWVEGTVGTLLSDITRLDLGKRILDP |
| | domain of | RGIYRCNGTDIYKDKESTVQVHYRMCQSCVELDPATVA |
| | human CD3δ | |
| 23 | transmembrane | GIIVTDVIATLLLALGVFCFA |
| | domain of | |
| | human CD3δ | |
| 24 | intracellular | GHETGRLSGAADTQALLRNDQVYQPLRDRDDAQYSHLGGNW |
| | domain of | ARNK |
| | human CD3δ | |
| 25 | human CD3-ζ | MKWKALFTAAILQAQLPITEAQSFGLLDPKLCYLLDGILFIYGVI |
| | | LTALFLRVKFSRSADAPAYQQGQNQLYNELNLGRREEYDVLDK |
| | | RRGRDPEMGGKPQRRKNPQEGLYNELQKDKMAEAYSEIGMKG |
| | | ERRRGKGHDGLYQGLSTATKDTYDALHMQALPPR |
| 26 | human TCR | MAGTWLLLLLALGCPALPTGVGGTPFPSLAPPIMLLVDGKQQM |
| | α-chain | VVVCLVLDVAPPGLDSPIWFSAGNGSALDAFTYGPSPATDGTW |
| | | TNLAHLSLPSEELASWEPLVCHTGPGAEGHSRSTQPMHLSGEAS |
| | | TARTCPQEPLRGTPGGALWLGVLRLLLFKLLLFDLLLTCSCLCD |
| | | PAGPLPSPATTTRLRALGSHRLHPATETGGREATSSPRPQPRDRR |
| | | WGDTPPGRKPGSPVWGEGSYLSSYPTCPAQAWCSRSALRAPSS |
| | | SLGAFFAGDLPPPLQAGA |
| 27 | human TCR | PNIQNPDPAVYQLRDSKSSDKSVCLFTDFDSQTNVSQSKDSDVY |
| | α-chain C | ITDKTVLDMRSMDFKSNSAVAWSNKSDFACANAFNNSIIPEDTF |
| | region | FPSPESSCDVKLVEKSFETDTNLNFQNLSVIGFRILLLKVAGFNL |
| | | LMTLRLWSS |
| 28 | human TCR | IQNPDPAVYQLRDSKSSDKSVCLFTDFDSQTNVSQSKDSDVYIT |
| | alpha chain | DKTVLDMRSMDFKSNSAVAWSNKSDFACANAFNNSIIPEDTFF |
| | human IgC | PSPESSCDVKLVEKSFETDTNLNFQNLS |
| 29 | human TCR | VIGFRILLLKVAGFNLLMTLRLW |
| | alpha chain | |
| | transmembrane | |
| | domain | |
| 30 | human TCR | SS |
| | alpha chain | |
| | intracellular | |
| | domain | |
| 31 | human TCR | MAMLLGASVLILWLQPDWVNSQQKNDDQQVKQNSPSLSVQEG |
| | α-chain V | RISILNCDYTNSMFDYFLWYKKYPAEGPTFLISISSIKDKNEDGR |
| | region CTL- | FTVFLNKSAKHLSLHIVPSQPGDSAVYFCAAKGAGTASKLTFGT |
| | L17 | GTRLQVTL |
| 32 | murine TCR | XIQNPEPAVYQLKDPRSQDSTLCLFTDFDSQINVPKTMESGTFIT |
| | alpha chain | DKTVLDMKAMDSKSNGAIAWSNQTSFTCQDIFKETNATYPSSD |
| | constant | VPCDATLTEKSFETDMNLNFQNLSVMGLRILLLKVAGFNLLMT |
| | (mTRAC) | LRLWSS |
| | region | |
| 33 | murine TCR | MGLRILLLKVAGFNLLMTLRLW |
| | alpha chain | |
| | transmembrane | |
| | domain | |
| 34 | murine TCR | SS |
| | alpha chain | |
| | intracellular | |
| | domain | |
| 35 | human TCR | EDLNKVFPPEVAVFEPSEAEISHTQKATLVCLATGFFPDHVELS |
| | β-chain C | WWVNGKEVHSGVSTDPQPLKEQPALNDSRYCLSSRLRVSATF |
| | region | WQNPRNHFRCQVQFYGLSENDEWTQDRAKPVTQIVSAEAWGR |
| | | ADCGFTSVSYQQGVLSATILYEILLGKATLYAVLVSALVLMAM |
| | | VKRKDF |
| 36 | human TCR | MGTSLLCWMALCLLGADHADTGVSQNPRHNITKRGQNVTFRC |
| | β-chain V | DPISEHNRLYWYRQTLGQGPEFLTYFQNEAQLEKSRLLSDRFSA |
| | region CTL- | ERPKGSFSTLEIQRTEQGDSAMYLCASSLAGLNQPQHFGDGTRL |
| | L17 | SIL |
| 37 | human TCR | MDSWTFCCVSLCILVAKHTDAGVIQSPRHEVTEMGQEVTLRCK |
| | β-chain V | PISGHNSLFWYRQTMMRGLELLIYFNNNVPIDDSGMPEDRFSAK |
| | region YT35 | MPNASFSTLKIQPSEPRDSAVYFCASSFSTCSANYGYTFGSGTRL |
| | | TVV |
| 38 | TCR beta | EDLNKVFPPEVAVFEPSEAEISHTQKATLVCLATGFFPDHVELS |
| | chain human | WWVNGKEVHSGVSTDPQPLKEQPALNDSRYCLSSRLRVSATF |
| | IgC | WQNPRNHFRCQVQFYGLSENDEWTQDRAKPVTQIVSAEAWGR |
| | | ADCGFTSVSYQQGVLSATILYE |
| 39 | human TCR | ILLGKATLYAVLVSALVLMAM |
| | beta chain | |
| | transmembrane | |
| | domain | |
| 40 | human TCR | VKRKDF |
| | beta chain | |
| | intracellular | |
| | domain | |
| 41 | murine TCR | EDLRNVTPPKVSLFEPSKAEIANKQKATLVCLARGFFPDHVELS |
| | beta chain | WWVNGKEVHSGVSTDPQAYKESNYSYCLSSRLRVSATFWHNP |
| | constant | RNHFRCQVQFHGLSEEDKWPEGSPKPVTQNISAEAWGRADCGI |
| | region | TSASYQQGVLSATILYEILLGKATLYAVLVSTLVVMAMVKRKN |
| | | S |
| 42 | murine TCR | ILYEILLGKATLYAVLVSTLVVMAMVK |
| | beta chain | |
| | transmembrane | |
| | domain | |
| 43 | murine TCR | KRKNS |
| | beta chain | |
| | intracellular | |
| | domain | |
| 44 | human TCR | DKQLDADVSPKPTIFLPSIAETKLQKAGTYLCLLEKFFPDVIKIH |
| | gamma chain | WQEKKSNTILGSQEGNTMKTNDTYMKFSWLTVPEKSLDKEHR |
| | constant | CIVRHENNKNGVDQEIIFPPIKTDVITMDPKDNCSKDANDTLLL |
| | region | QLTNTSAYYMYLLLLLKSVVYFAIITCCLLRRTAFCCNGEKS |
| 45 | human TCR | DKQLDADVSPKPTIFLPSIAETKLQKAGTYLCLLEKFFPDVIKIH |
| | gamma IgC | WQEKKSNTILGSQEGNTMKTNDTYMKFSWLTVPEKSLDKEHR |
| | | CIVRHENNKNGVDQEIIFPPIKTDVITMDPKDNCSKDANDTLLL |
| | | QLTNTSA |
| 46 | human TCR | YYMYLLLLLKSVVYFAIITCCLL |
| | gamma chain | |
| | transmembrane | |
| | domain | |
| 47 | human TCR | RRTAFCCNGEKS |
| | gamma chain | |
| | intracellular | |
| | domain | |
| 48 | human TCR | SQPHTKPSVFVMKNGTNVACLVKEFYPKDIRINLVSSKKITEFDP |
| | delta chain | AIVISPSGKYNAVKLGKYEDSNSVTCSVQHDNKTVHSTDFEVK |
| | constant | TDSTDHVKPKETENTKQPSKSCHKPKAIVHTEKVNMMSLTVLG |
| | region | LRMLFAKTVAVNFLLTAKLFFL |
| 49 | human TCR | SQPHTKPSVFVMKNGTNVACLVKEFYPKDIRINLVSSKKITEFDP |
| | delta IgC | AIVISPSGKYNAVKLGKYEDSNSVTCSVQHDNKTVHSTDFEVK |
| | | TDSTDHVKPKETENTKQPSKSCHKPKAIVHTEKVNMMSLTV |
| 50 | human TCR | LGLRMLFAKTVAVNFLLTAKLFF |
| | delta chain | |
| | transmembrane | |
| | domain | |
| 51 | human TCR | L |
| | delta chain | |
| | intracellular | |
| | domain | |
| 52 | MH1 TFP | MLLLVTSLLLCELPHPAFLLIPEVQLVESGGGLVQPGGSLRLSCA |
| | | ASGGDWSANFMYWYRQAPGKQRELVARISGRGVVDYVESVK |
| | | GRFTISRDNSKNTLYLQMNSLRAEDTAVYYCAVASYWGQGTL |
| | | VTVSSAAAGGGGSGGGGSGGGGSLEDGNEEMGGITQTPYKVSI |
| | | SGTTVILTCPQYPGSEILWQHNDKNIGGDEDDKNIGSDEDHLSL |
| | | KEFSELEQSGYYVCYPRGSKPEDANFYLYLRARVCENCMEMD |
| | | VMSVATIVIVDICITGGLLLLVYYWSKNRKAKAKPVTRGAGAG |
| | | GRQRGQNKERPPPVPNPDYEPIRKGQRDLYSGLNQRRI |
| 53 | MH1ε TFP | MLLLVTSLLLCELPHPAFLLIPEVQLVESGGGLVQPGGSLRLSCA |
| | T2A Foxp3 | ASGGD |
| | | WSANFMYWYRQAPGKQRELVARISGRGVVDYVESVKGRFTIS |
| | | RDNSKNTL |
| | | YLQMNSLRAEDTAVYYCAVASYWGQGTLVTVSSAAAGGGGS |
| | | GGGGSGGGG |
| | | SLEDGNEEMGGITQTPYKVSISGTTVILTCPQYPGSEILWQHNDK |
| | | NIGGD |
| | | EDDKNIGSDEDHLSLKEFSELEQSGYYVCYPRGSKPEDANFYLY |
| | | LRARVC |
| | | ENCMEMDVMSVATIVIVDICITGGLLLLVYYWSKNRKAKAKPV |
| | | TRGAGAG |
| | | GRQRGQNKERPPPVPNPDYEPIRKGQRDLYSGLNQRRIGSGEGR |
| | | GSLLTC |
| | | GDVEENPGPMPNPRPGKPSAPSLALGPSPGASPSWRAAPKASDL |
| | | LGARGP |
| | | GGTFQGRDLRGGAHASSSSLNPMPPSQLQLPTLPLVMVAPSGA |
| | | RLGPLPH |
| | | LQALLQDRPHFMHQLSTVDAHARTPVLQVHPLESPAMISLTPPT |
| | | TATGVF |
| | | SLKARPGLPPGINVASLEWVSREPALLCTFPNPSAPRKDSTLSAV |
| | | PQSSY |
| | | PLLANGVCKWPGCEKVFEEPEDFLKHCQADHLLDEKGRAQCL |
| | | LQREMVQS |
| | | LEQQLVLEKEKLSAMQAHLAGKMALTKASSVASSDKGSCCIVA |
| | | AGSQGPV |
| | | VPAWSGPREAPDSLFAVRRHLWGSHGNSTFPEFLHNMDYFKFH |
| | | NMRPPFT |
| | | YATLIRWAILEAPEKQRTLNEIYHWFTRMFAFFRNHPATWKNAI |
| | | RHNLSL |
| | | HKCFVRVESEKGAVWTVDELEFRKKRSQRPSRCSNPTPGP |
| 54 | 3PF12ε T2A | MLLLVTSLLLCELPHPAFLLIPQVQLVQSGGGVVQPGGSLRVSC |
| | Foxp3 | AASGVT |
| | | LSDYGMHWVRQAPGKGLEWMAFIRNDGSDKYYADSVKGRFTI |
| | | SRDNSKKT |
| | | VSLQMSSLRAEDTAVYYCAKNGESGPLDYWYFDLWGRGTLVT |
| | | VSSGGGGS |
| | | GGGGSGGGGSDVVMTQSPSSLSASVGDRVTITCQASQDISNYL |
| | | NWYQQKP |
| | | GKAPKLLIYDASNLETGVPSRFSGSGSGTDFTFTISSLQPEDFAT |
| | | YYCQQ |
| | | YSSFPLTFGGGTKVDIKRAAAGGGGSGGGGSGGGGSLEDGNEE |
| | | MGGITQT |
| | | PYKVSISGTTVILTCPQYPGSEILWQHNDKNIGGDEDDKNIGSDE |
| | | DHLSL |
| | | KEFSELEQSGYYVCYPRGSKPEDANFYLYLRARVCENCMEMD |
| | | VMSVATIV |
| | | IVDICITGGLLLLVYYWSKNRKAKAKPVTRGAGAGGRQRGQN |
| | | KERPPPVP |
| | | NPDYEPIRKGQRDLYSGLNQRRIGSGEGRGSLLTCGDVEENPGP |
| | | GMPNPR |
| | | PGKPSAPSLALGPSPGASPSWRAAPKASDLLGARGPGGTFQGRD |
| | | LRGGAH |
| | | ASSSSLNPMPPSQLQLPTLPLVMVAPSGARLGPLPHLQALLQDR |
| | | PHFMHQ |
| | | LSTVDAHARTPVLQVHPLESPAMISLTPPTTATGVFSLKARPGLP |
| | | PGINV |
| | | ASLEWVSREPALLCTFPNPSAPRKDSTLSAVPQSSYPLLANGVC |
| | | KWPGCE |
| | | KVFEEPEDFLKHCQADHLLDEKGRAQCLLQREMVQSLEQQLVL |
| | | EKEKLSA |
| | | MQAHLAGKMALTKASSVASSDKGSCCIVAAGSQGPVVPAWSG |
| | | PREAPDSL |
| | | FAVRRHLWGSHGNSTFPEFLHNMDYFKFHNMRPPFTYATLIRW |
| | | AILEAPE |
| | | KQRTLNEIYHWFTRMFAFFRNHPATWKNAIRHNLSLHKCFVRV |
| | | ESEKGAV |
| | | WTVDELEFRKKRSQRPSRCSNPTPGP |
| 55 | 3PF12ε T2A | MLLLVTSLLLCELPHPAFLLIPQVQLVQSGGGVVQPGGSLRVSC |
| | Foxp3 P2A | AASGVT |
| | trEGFR | LSDYGMHWVRQAPGKGLEWMAFIRNDGSDKYYADSVKGRFTI |
| | | SRDNSKKT |
| | | VSLQMSSLRAEDTAVYYCAKNGESGPLDYWYFDLWGRGTLVT |
| | | VSSGGGGS |
| | | GGGGSGGGGSDVVMTQSPSSLSASVGDRVTITCQASQDISNYL |
| | | NWYQQKP |
| | | GKAPKLLIYDASNLETGVPSRFSGSGSGTDFTFTISSLQPEDFAT |
| | | YYCQQ |
| | | YSSFPLTFGGGTKVDIKRAAAGGGGSGGGGSGGGGSLEDGNEE |
| | | MGGITQT |
| | | PYKVSISGTTVILTCPQYPGSEILWQHNDKNIGGDEDDKNIGSDE |
| | | DHLSL |
| | | KEFSELEQSGYYVCYPRGSKPEDANFYLYLRARVCENCMEMD |
| | | VMSVATIV |
| | | IVDICITGGLLLLVYYWSKNRKAKAKPVTRGAGAGGRQRGQN |
| | | KERPPPVP |
| | | NPDYEPIRKGQRDLYSGLNQRRIGSGEGRGSLLTCGDVEENPGP |
| | | GMPNPR |
| | | PGKPSAPSLALGPSPGASPSWRAAPKASDLLGARGPGGTFQGRD |
| | | LRGGAH |
| | | ASSSSLNPMPPSQLQLPTLPLVMVAPSGARLGPLPHLQALLQDR |
| | | PHFMHQ |
| | | LSTVDAHARTPVLQVHPLESPAMISLTPPTTATGVFSLKARPGLP |
| | | PGINV |
| | | ASLEWVSREPALLCTFPNPSAPRKDSTLSAVPQSSYPLLANGVC |
| | | KWPGCE |
| | | KVFEEPEDFLKHCQADHLLDEKGRAQCLLQREMVQSLEQQLVL |
| | | EKEKLSA |
| | | MQAHLAGKMALTKASSVASSDKGSCCIVAAGSQGPVVPAWSG |
| | | PREAPDSL |
| | | FAVRRHLWGSHGNSTFPEFLHNMDYFKFHNMRPPFTYATLIRW |
| | | AILEAPE |
| | | KQRTLNEIYHWFTRMFAFFRNHPATWKNAIRHNLSLHKCFVRV |
| | | ESEKGAV |
| | | WTVDELEFRKKRSQRPSRCSNPTPGPGSGATNFSLLKQAGDVEE |
| | | NPGPML |
| | | LLVTSLLLCELPHPAFLLIPRKVCNGIGIGEFKDSLSINATNIKHF |
| | | KNCT |
| | | SISGDLHILPVAFRGDSFTHTPPLDPQELDILKTVKEITGFLLIQA |
| | | WPEN |
| | | RTDLHAFENLEIIRGRTKQHGQFSLAVVSLNITSLGLRSLKEISD |
| | | GDVII |
| | | SGNKNLCYANTINWKKLFGTSGQKTKIISNRGENSCKATGQVC |
| | | HALCSPE |
| | | GCWGPEPRDCVSCRNVSRGRECVDKCNLLEGEPREFVENSECI |
| | | QCHPECL |
| | | PQAMNITCTGRGPDNCIQCAHYIDGPHCVKTCPAGVMGENNTL |
| | | VWKYADA |
| | | GHVCHLCHPNCTYGCTGPGLEGCPTNGPKIPSIATGMVGALLLL |
| | | LVVALG |
| | | IGLFM |
| 60 | anti-MSLN | GGDWSANFMY |
| | (SD1) CDR1 | |
| 61 | anti-MSLN | RISGRGVVDYVESVKGRFT |
| | (SD1) CDR2 | |
| 62 | anti-MSLN | ASY |
| | (SD1) CDR3 | |
| 63 | anti-MSNL | GSTSSINTMY |
| | (SD4) CDR1 | |
| 64 | anti-MSNL | FISSGGSTNVRDSVKGRFT |
| | (SD4) CDR2 | |
| 65 | anti-MSNL | YIPYGGTLHDF |
| | (SD4) CDR3 | |
| 66 | anti-MSNL | GSTFSIRAMR |
| | (SD6) CDR1 | |
| 67 | anti-MSNL | VIYGSSTYYADAVKGRFT |
| | (SD6) CDR2 | |
| 68 | anti-MSNL | DTIGTARDY |
| | (SD6) CDR3 | |
| 69 | Single domain | EVQLVESGGGLVQPGGSLRLSCAASGGDWSANFMYWYRQAPG |
| | anti-MSLN | KQRELVARISGRGVVDYVESVKGRFTISRDNSKNTLYLQMNSL |
| | binder 1 | RAEDTAVYYCAVASYWGQGTLVTVSS |
| | (SD1) | |
| 70 | Single domain | EVQLVESGGGLVQPGGSLRLSCAASGSTSSINTMYWYRQAPGK |
| | anti-MSLN | ERELVAFISSGGSTNVRDSVKGRFTISRDNSKNTLYLQMNSLRA |
| | binder 4 | EDTAVYYCNTYIPYGGTLHDFWGQGTLVTVSS |
| | (SD4) | |
| 71 | Single domain | QVQLVESGGGVVQAGGSLRLSCAASGSTFSIRAMRWYRQAPGT |
| | anti-MSLN | ERDLVAVIYGSSTYYADAVKGRFTISRDNSKNTLYLQMNSLRA |
| | binder 6 | EDTAVYYCNADTIGTARDYWGQGTLVTVSS |
| | (SD6) | |

### TFP Expression Vectors

Expression vectors are provided that include: a promoter (an EF1alpha promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

TFP-encoding nucleic acid construct was cloned into a lentiviral expression vector.

### Example 2: Preparation of T-Cells Transduced with TFPs

### Lentiviral Production

Lentivirus encoding the appropriate constructs were prepared as follows. Expi293F-cells are suspended in FS media and allowed to incubate at 37 degrees C., 8% CO₂, 150 rpm for 1-3 hours. The transfer DNA plasmid, Gag/Pol plasmid, Rev plasmid, and VSV-G plasmid were diluted in FS media. PEIpro was then diluted in FS media and added to the mixture of DNA and media. The incubated cells were added to this mixture and were incubated at 37 degrees C., 8% CO₂, 150 rpm for 18-24 hours. The following day, the supernatant was replaced with fresh media and supplemented with sodium butyrate and incubated at 37° C. for an additional 24 hours. The lentivirus containing supernatant was then collected into a 50 mL sterile, capped conical centrifuge tube and put on ice. After centrifugation at 3000 rpm for 30 minutes at 4° C., the cleared supernatant was filtered with a low-protein binding 0.45 m sterile filter. The virus was subsequently concentrated by Lenti-X. The virus stock preparation was either used for infection immediately or aliquoted and stored at −80° C. for future use.

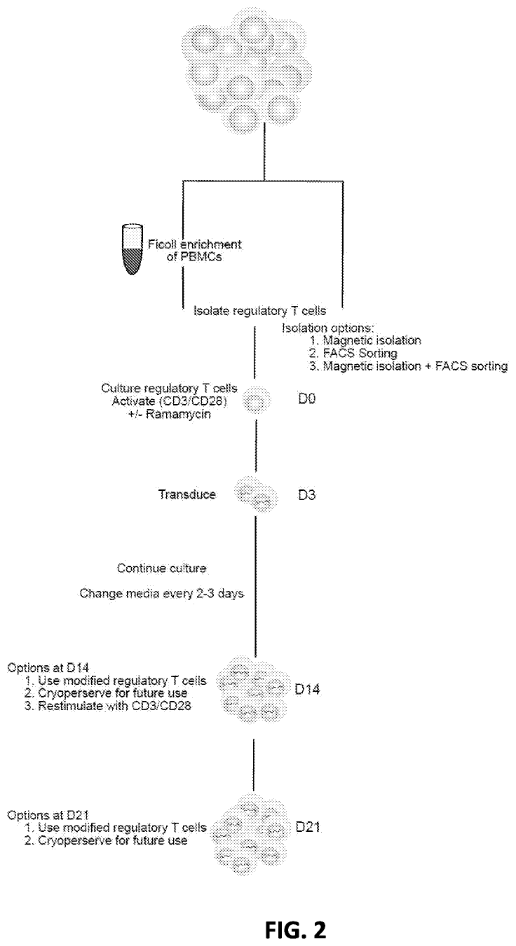

FIG. 2 shows a schematic of the process for producing TFP-expressing Treg.

### T Regulatory Cell Activation, Transfection, and Expansion

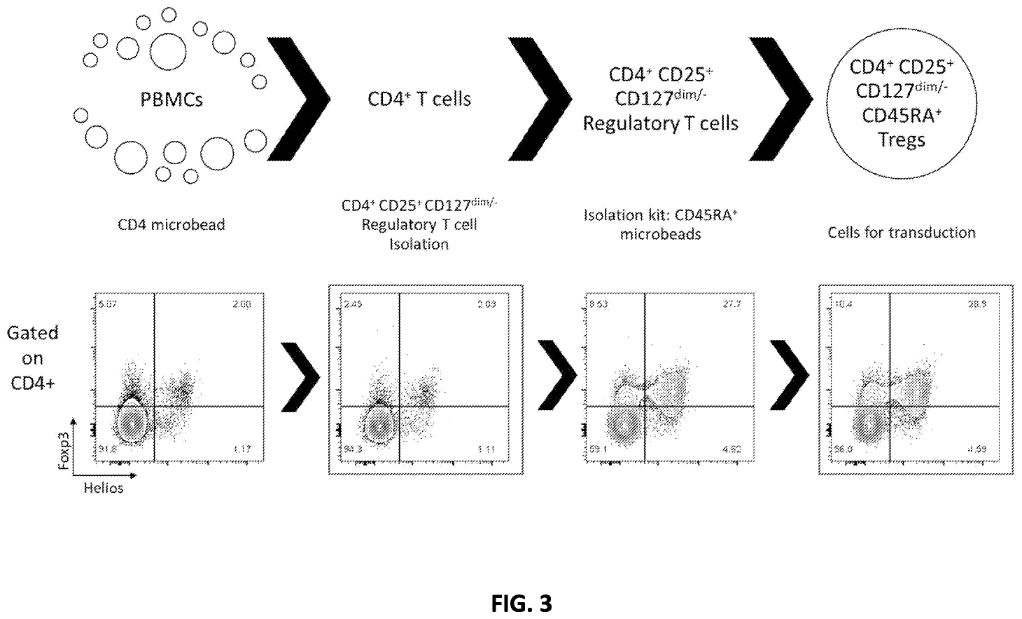

T regulatory cells and CD4+ T cells were isolated from peripheral blood either directly or from peripheral blood mononuclear cells (PBMCs) enriched on a Ficoll gradient. Total CD4+ T cells were isolated with the REAlease CD4 microbead. A portion of these cells were taken and used as the CD4+ cells in the experiments below while the remaining cells were used to further isolate Treg. For Treg isolation, CD4+ CD25+ CD127dim/−Treg were then isolated from the CD4+ cells using a CD4+ CD25+ CD127dim/−isolating kit (Miltenyi). CD4+ CD25+ CD127dim/− CD45RA+ were then isolated from the CD4+ CD25+ CD127dim/− Treg using a CD45 microbead kit to generate the Treg used in the experiments described below. The Treg isolation scheme and the enrichment of FoxP3+ Helios+ cells at each step of the process is shown in FIG. 3.

For CD4+ cells, On day 0, isolated CD4+ T cells, were activated by MACS GMP T cell TransAct (Miltenyi Biotech), in X-Vivo media+10% FBS+12.5 ng/ml IL7/IL15. On day 1, activated T cells were transduced with lentivirus encoding the HLA-A2 TFP or MH1-TFP (with or without FoxP3). On days 4, 7 and 10, the cells were washed, subcultured in fresh medium with cytokines and then expanded up to day 14.

For Treg cells, On day 0, Treg were isolated from PBMC (that had been thawed and rested the previous day) and were activated by MACS GMP T cell TransAct (Miltenyi Biotech), in X-Vivo media+10% FBS+1000 IU/ml IL-2+100 nM Rapamycin. On day 1, activated T cells were transduced with lentivirus encoding the HLA-A2 TFP or MH1-TFP (with or without FoxP3). On days 4, 7 and 10, the cells were washed, subcultured in fresh medium with cytokines and then expanded up to day 14. At each day of subculture, cells were harvested, washed, and resuspended with fresh cytokine-containing medium.

### Example 3: Antigen Independent Suppression Assays

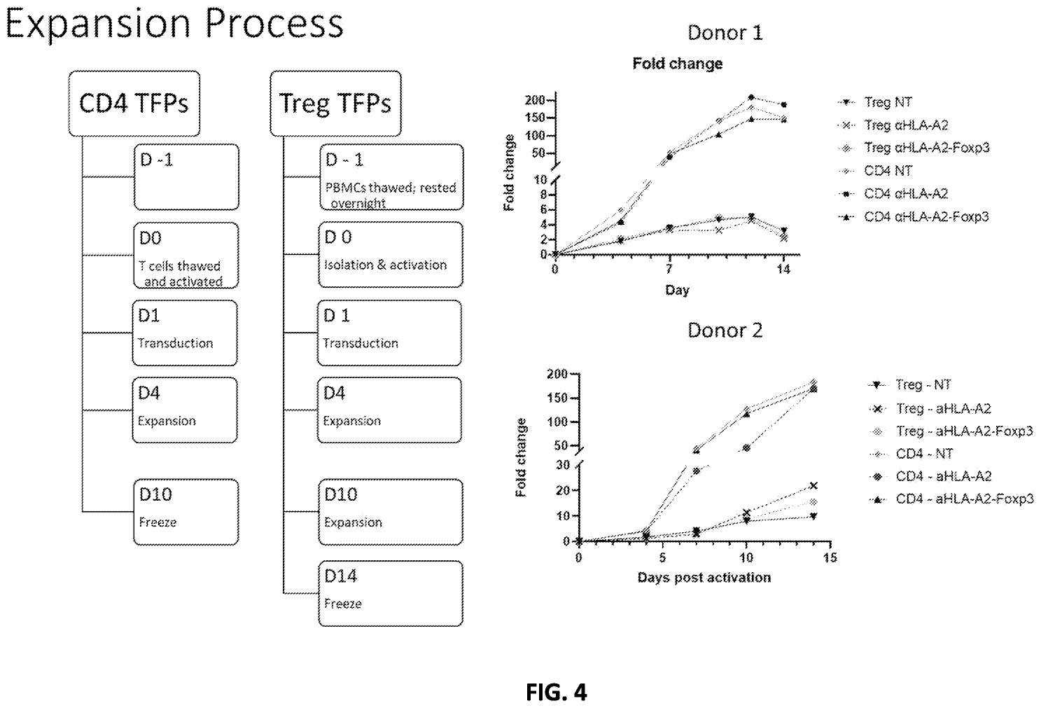

CD4+ and regulatory T cells having the HLA-A2 TFP with or without FoxP3 prepared as described in Example 2 were used in an antigen independent suppression assay. All constructs contained a truncated EGFR. The expansion process for Treg and CD4+ T cells having the HLA-A2 TFP with or without FoxP3 and the expansion rates of transduced and non-transduced CD4 and Treg cells are shown in FIG. 4. Tregs and CD4+ T cells were isolated, transduced, and expanded from two separate donors (Donor 1 and Donor 2).

Verification of TFP expression and Phenotyping of TFP T Cells

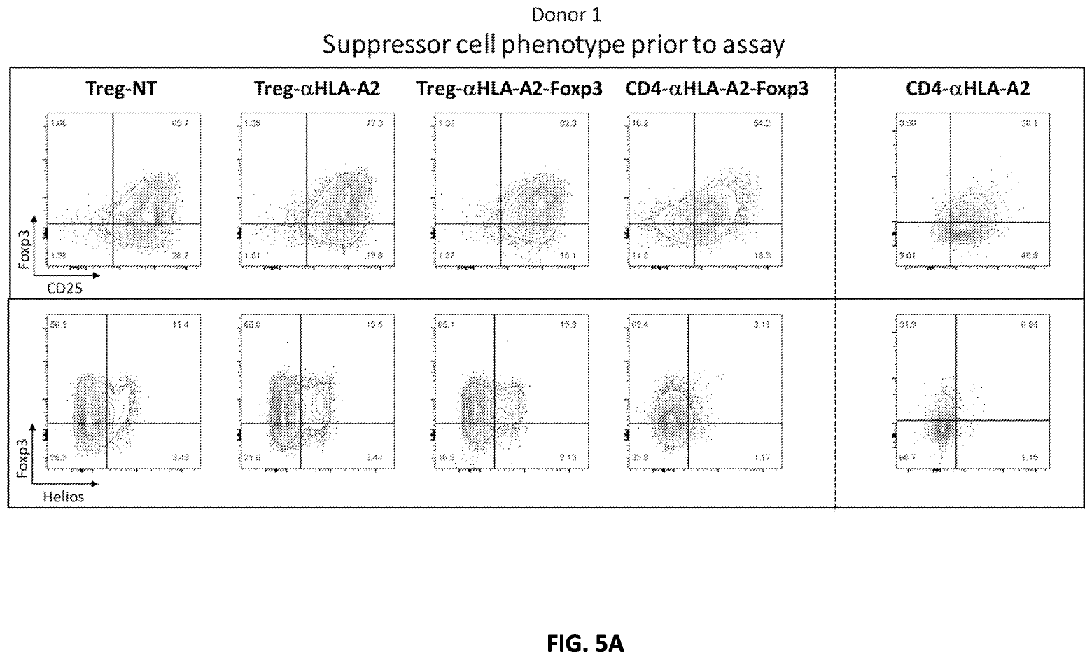

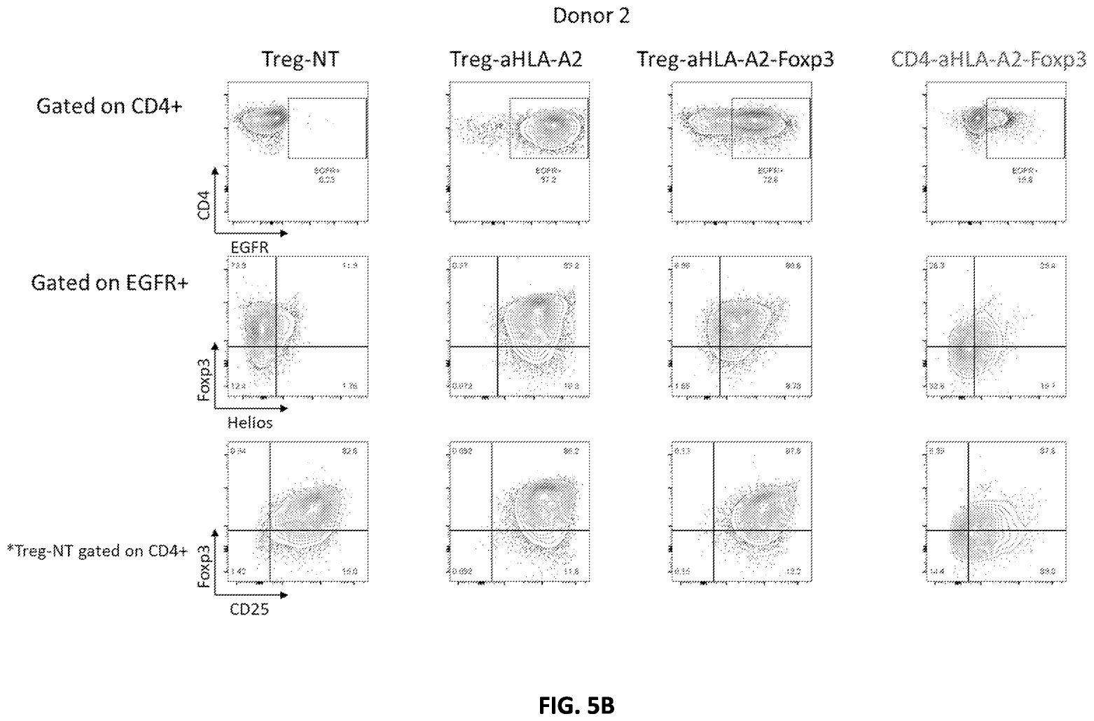

Phenotyping of the HLA-A2 TFP transduced T cells was performed. TFP T cells or non-transduced T cells were generated as described above. At day 14 of expansion T cells were harvested and the cells were characterized by flow cytometry for expression of FoxP3, CD25, and Helios. Verification of TFP expression was also confirmed in cells by anti-EGFR staining. As is shown in FIGS. 5A and 5B, nontransduced Treg, HLA-A2 TFP Treg, with and without the added FoxP3 gene, and HLA-A2 TFP CD4+ cells with the added FoxP3 gene have high levels of FoxP3 expression (FIGS. 5A and 5B). While all populations of Treg (nontransduced and transduced) have significant Helios (FIGS. 5A and 5B) and CD25 (FIG. 5B) expression, little Helios or CD25 expression is seen in HLA-A2 TFP CD4+ cells, even with the added FoxP3 gene. As is shown in FIG. 5B, while a high proportion of HLA-A2 TFP CD4+ cells (with or without FoxP3) show EGFR staining, a lower proportion of HLA-A2 TFP CD4+ cells with the added FoxP3 gene show EGFR staining, suggesting lower transduction efficiency in these cells.

### Antigen Independent Suppression Assay

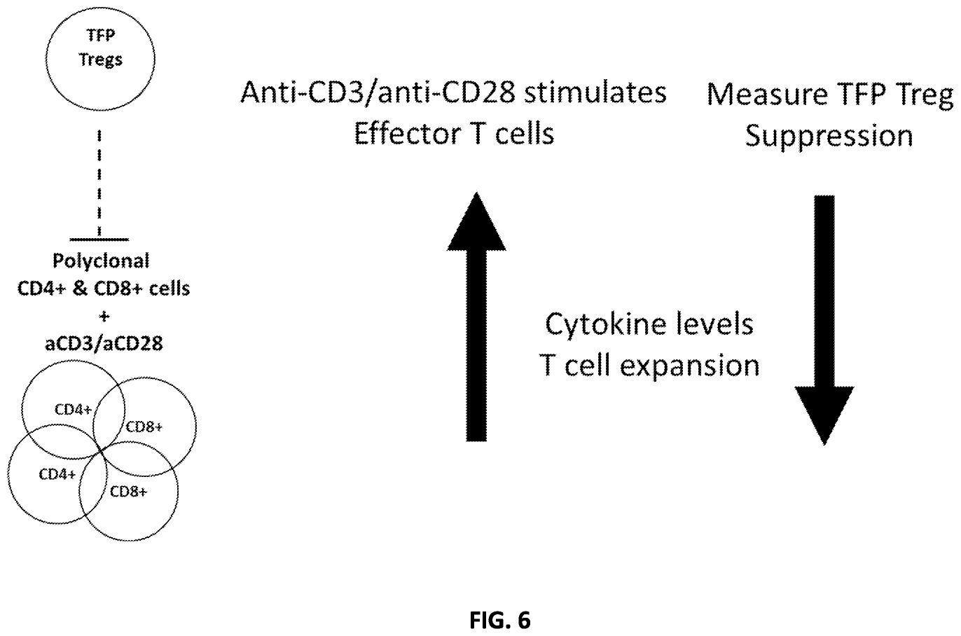

A suppression assay was performed to test the efficacy of HLA-A2 TFP regulatory T cells in suppressing effector T cells using a Treg Suppression Inspector Kit (Miltenyi). Polyclonal CD4+ and CD8+ T cells labeled with cell trace violet (CTV) were activated via the T cell receptor with anti-CD3 and anti-CD28 antibody beads (responder cells) and mixed with TFP.anti-HLA-A2 Treg with or without FoxP3, TFP.anti-HLA-A2 CD4+ T cells with FoxP3, or control Treg that do not express a TFP at various concentrations. Supernatants were then taken for cytokine analysis and flow analysis was done to assess expansion of responder cells after 72 hours of incubation. A schematic of the assay is shown in FIG. 6.

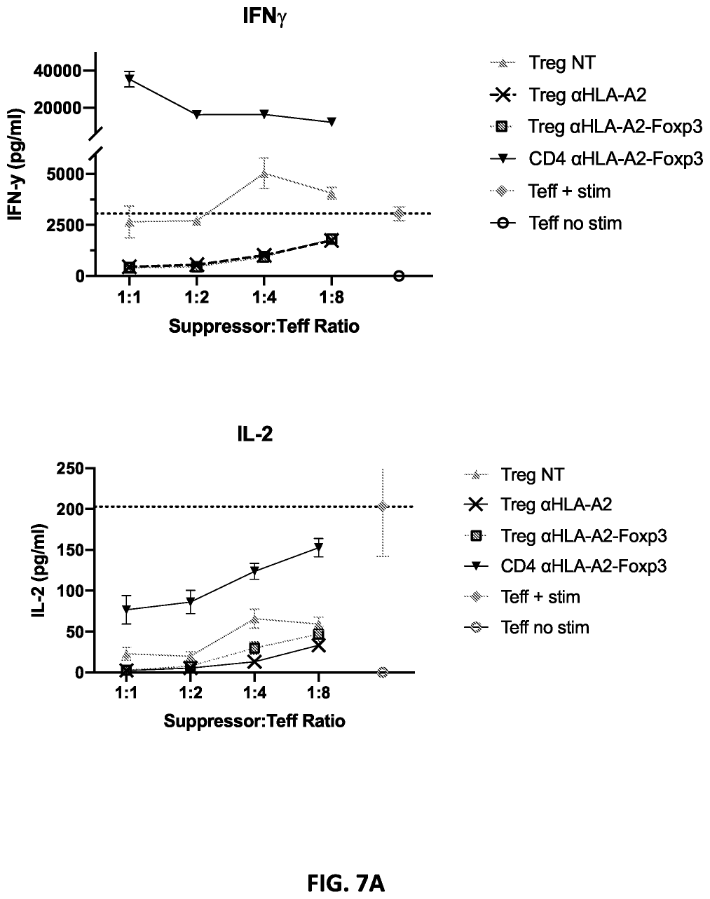

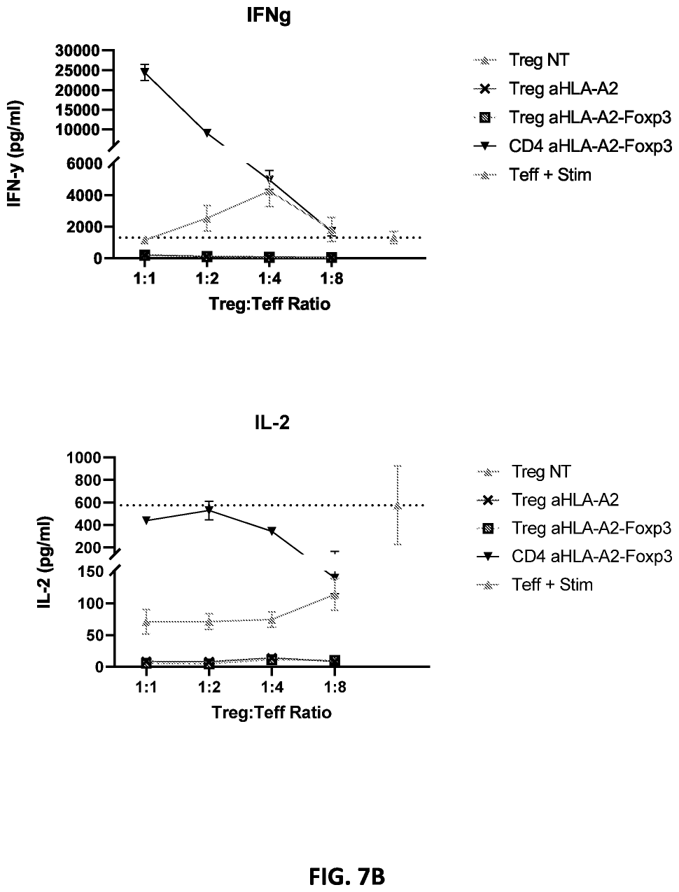

For cytokine analysis, levels of IFN-gamma and IL-2 were measured at each of the suppressor TFP T cell:responder cell ratios. As is shown in FIG. 7A (Donor 1) and 7B (Donor 2), for both IFN-gamma and IL-2, HLA-A2 TFP Treg with or without the added FoxP3 gene were able to suppress IFN-gamma and IL-2 production of the responder cells (relative to the level of cytokines produced by activated responder cells not treated with Treg or CD4+ T cells). For both donors, the suppression achieved was beyond that of untransduced Treg. Treating the responder cells with HLA-A2 TFP CD4+ cells with the added FoxP3 gene increased IFN-gamma expression beyond that of no treatment. This suggests that HLA-A2 TFP Treg can suppress cytokine production by effector T cells in an antigen independent manner.

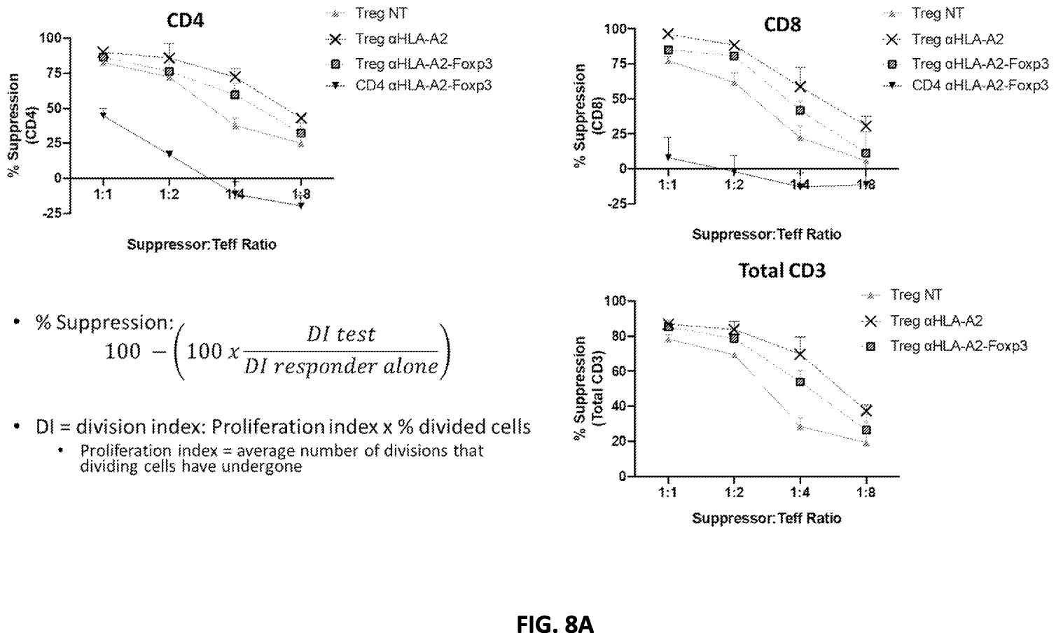

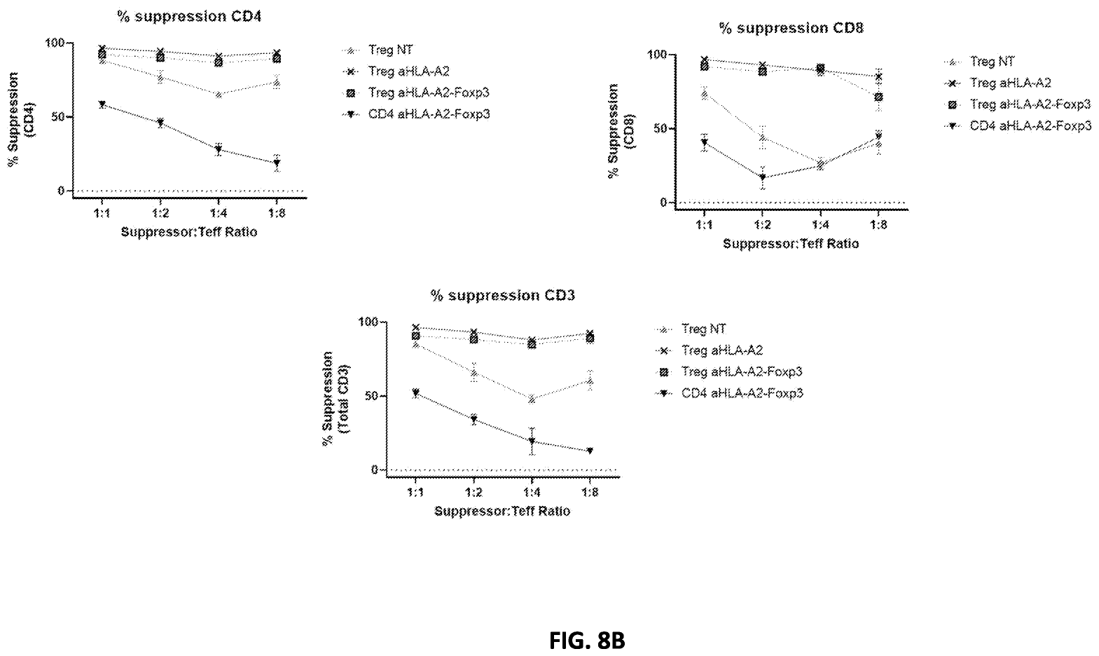

Expansion rate of the responder cells was also evaluated by FACS at each of the TFP T cell:responder cell ratios by quantifying the % suppression, as is calculated by the formula shown in FIG. 8A (Donor1) and 8B (Donor 2). As is shown in FIG. 8, HLA-A2 TFP Treg with or without the added FoxP3 suppressed expansion of CD4+ responder T cells, CD8+ responder T cells, and total CD3+ responder T cells above non-transduced Treg for both donors. HLA-A2 TFP CD4+ T cells with the added FoxP3 gene were not able to suppress cell expansion as well as any of these cell populations, including non-transduced T cells, and only showed suppression activity at high suppressor cell:responder ratios. This suggests that HLA-A2 TFP Treg can suppress effector T cell expansion in an antigen independent manner.

### Example 4: Antigen Dependent Suppression Assays

CD4+ and regulatory T cells having the MH1 TFP with or without FoxP3 were used in an antigen dependent suppression assay.

### Verification of TFP Expression by Cell Staining

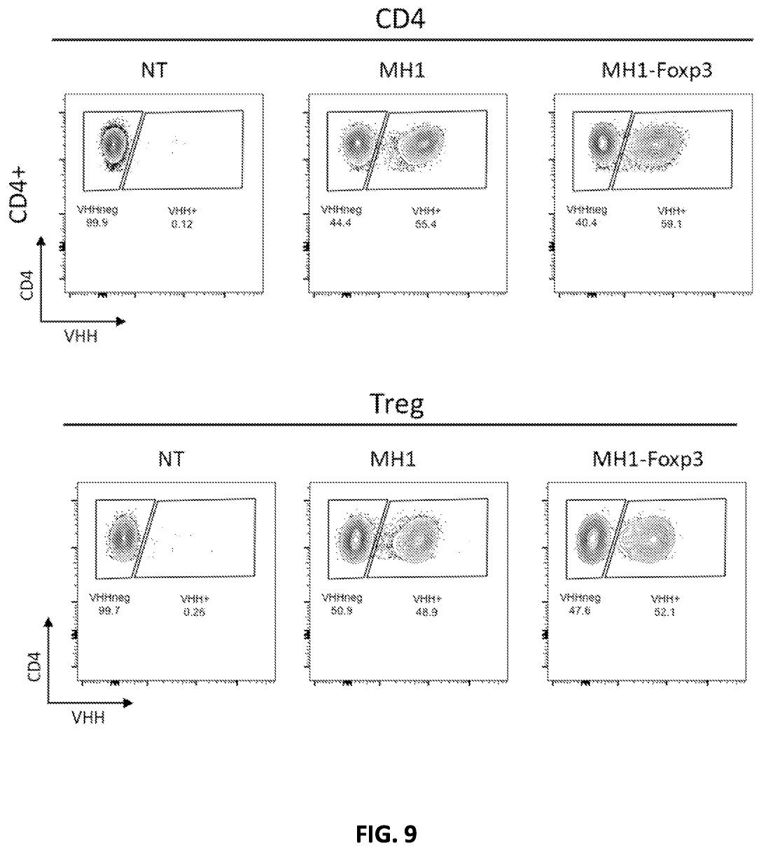

Following lentiviral transduction, expression of MH1TFP was confirmed by flow cytometry. Cells were stained for surface markers using anti-VHH and anti-CD4 antibodies. For dead cell exclusion, cells were incubated with LIVE/DEAD® Fixable Aqua Dead Cell Stain (Invitrogen). Flow cytometry was performed using LSRFortessa™ X20 (BD Biosciences) and data was acquired using FACS Diva software and was analyzed with FlowJo® (Treestar, Inc. Ashland, OR). As is shown in FIG. 9, CD4+ T cells and Treg transduced with the MH1 TFP or the MH1 TFP with the FoxP3 gene both expressed MH1 TFP.

### Phenotyping of TFP T Cells

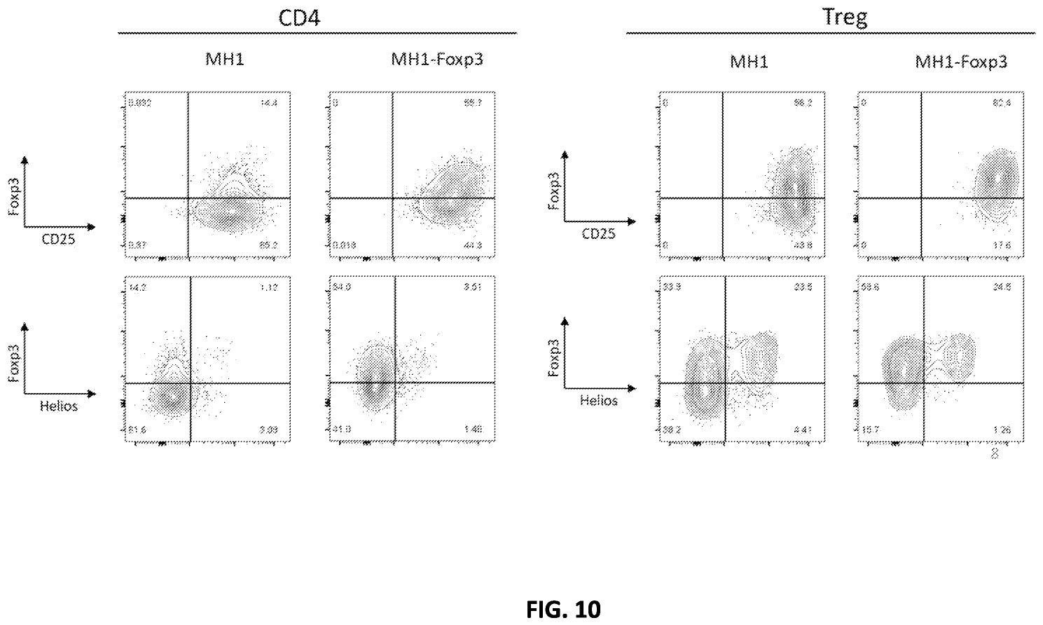

Phenotyping of the MH1 TFP transduced T cells was performed. TFP T cells or non-transduced T cells were generated as described above. At day 14 of expansion CD4+ and Treg cells were harvested and the cells were characterized by flow cytometry for expression of FoxP3, CD25, and Helios. As is shown in FIG. 10, MH1 TFP Treg, with and without the added FoxP3 gene, and MH1 CD4+ cells with the added FoxP3 gene have high levels of FoxP3 expression. While MH1 TFP Treg (with and without added FoxP3) have significant Helios expression, little Helios expression is seen in MH1 TFP CD4+ cells, even with the added FoxP3 gene.

### MH1 Antigen Dependent Suppression Assay

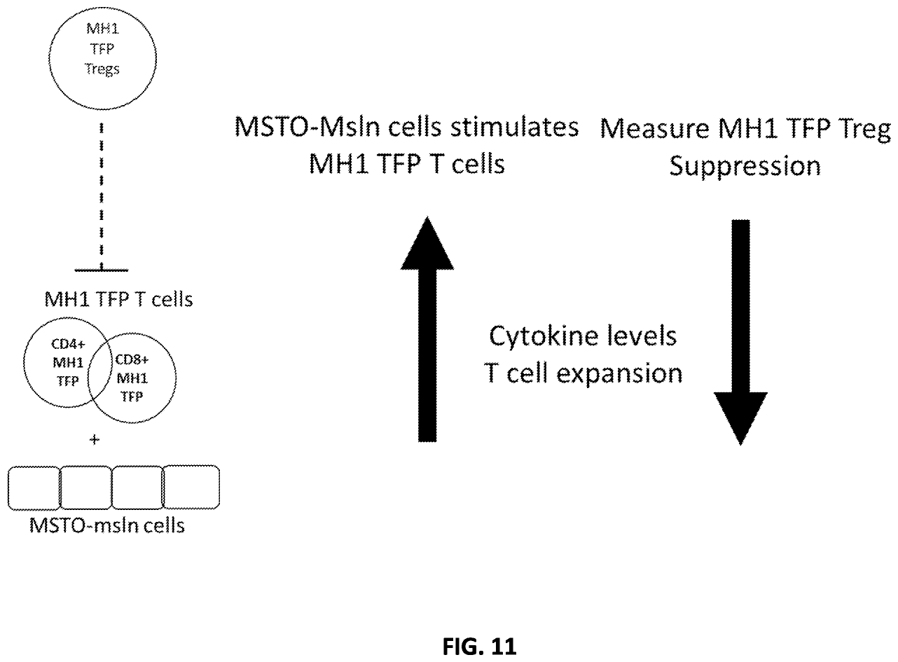

A suppression assay was performed to test the efficacy of MH1 TFP regulatory T cells in suppressing MH1 TFP effector T cells (TC-210). MH1 TFP effector cells were generated according to previously described methods for generating effector TFP+ T cells. (MH1 TFP effector T cells labeled with cell trace violet (CTV) (responder cells) were plated at a concentration of 50,000 cells/well on a layer of MSTO-msln cells. The MSTO-msln cells were plated at a concentration of 6,250 cells/well resulting in a MH1 TFP effector T cell:MSTO-msln cell ratio of 8:1. The MSTO-msln cells activate the MH1 TFP effector T cells in an antigen specific manner. MH1 TFP regulatory T cells with or without FoxP3, or CD4+ MH1 TFP T cells with Fox P3 were then added to the MH1 TFP effector T at various concentrations to measure the suppressive effect of the MH1 TFP Treg or CD4+ cells on the MH1 TFP effector T cells. Supernatants were taken for cytokine analysis and flow analysis was done on the responder cells to assess expansion after 72 hours of incubation. A schematic of the assay is shown in FIG. 11.

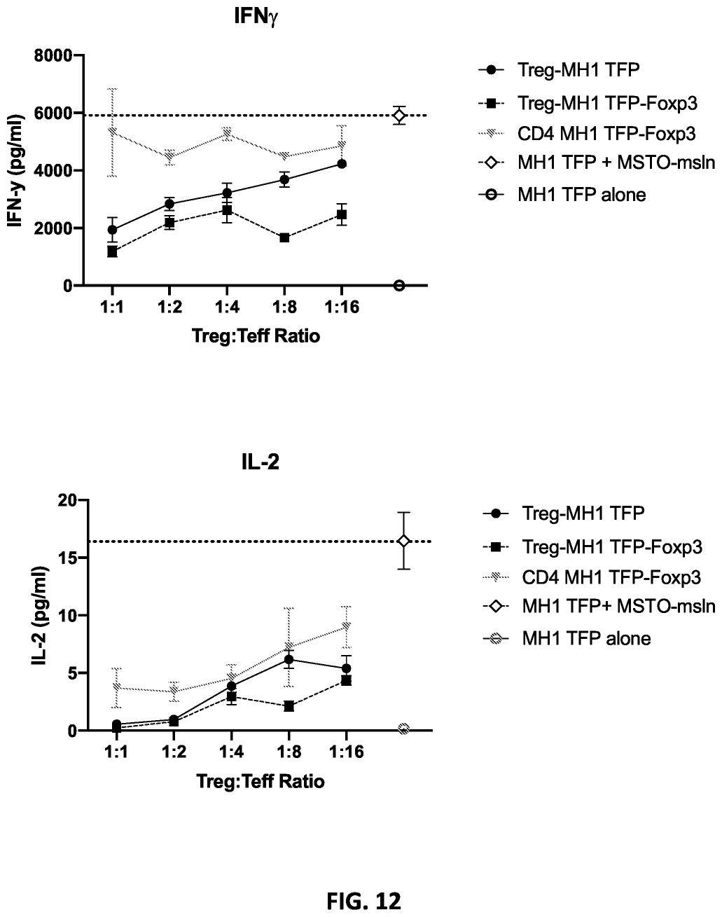

For cytokine analysis, levels of IFN-gamma and IL-2 were measured at each of the suppressor TFP T cell:responder cell ratios. As is shown in FIG. 12, for both IFN-gamma and IL-2, MH1 TFP Treg with or without the added FoxP3 gene were able to suppress IFN-gamma and IL-2 production of the responder cells (relative to the level of cytokines produced by activated responder cells not treated with Treg or CD4+ T cells). In contrast, treating the responder cells with MH1 TFP CD4+ cells with the added FoxP3 gene had less of a suppressive effect on cytokine production. Indeed, MH1 TFP CD4+ FoxP3+ cells were not able to suppress production of IFN-gamma at any ratio. This suggests that MH1 TFP Treg can suppress cytokine production by effector T cells in an antigen-specific manner.

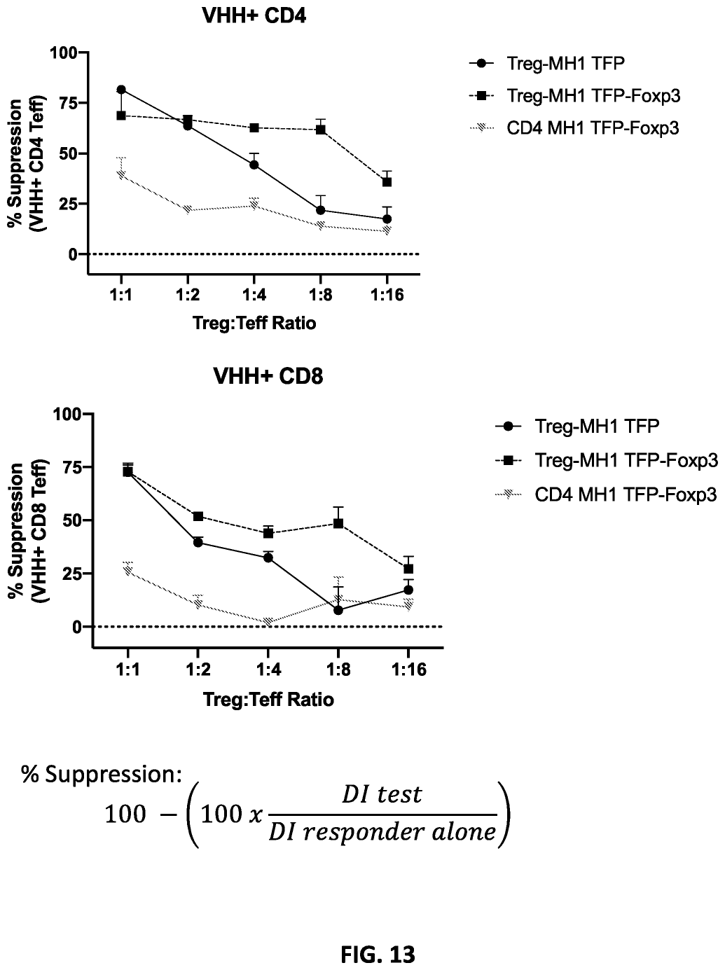

Expansion rate of the responder cells was also evaluated by FACS at each of the TFP T cell:responder cell ratios by quantifying the % suppression, as is calculated by the formula shown in FIG. 13. As is shown in FIG. 13, MH1 TFP Treg with or without the added FoxP3 gene were able to suppress expansion of MI TFP T CD4+ effector T cells and MH1 TFP T CD8+ effector T cells and the level of suppression was above that achieved by MH1 TFP CD4+ T cells with the added FoxP3 gene. Indeed, MH1 TFP CD4+ FoxP3+ cells were only able to suppress expansion at the highest suppressor:responder cell ratios. This suggests that MH1 TFP Treg can suppress effector T cell expansion in an antigen dependent manner.

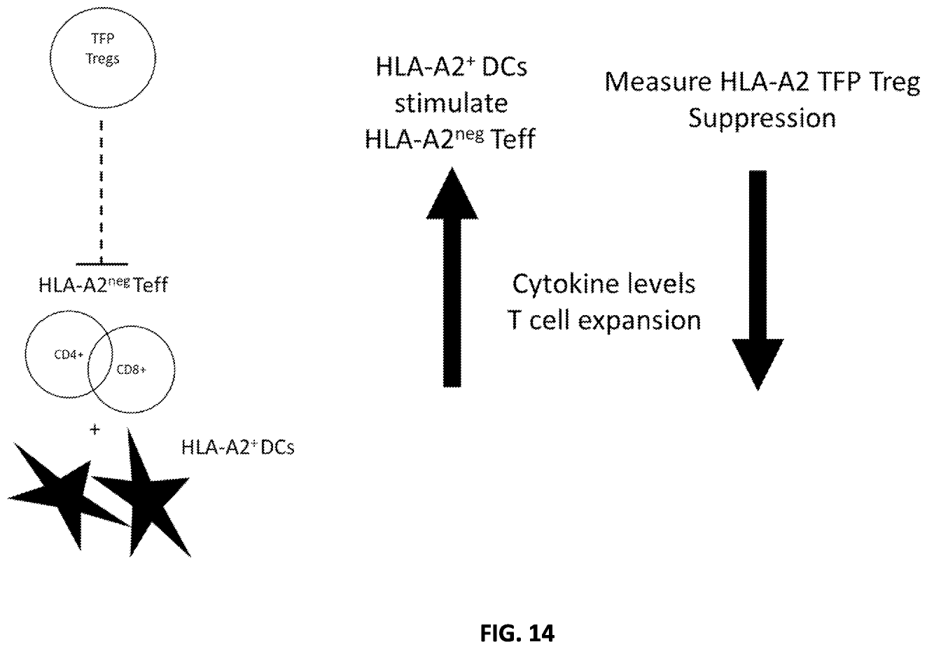

HLA-A2 Antigen Dependent Suppression AssayCD4+ and regulatory T cells having the HLA-A2 TFP with or without FoxP3 prepared as described in Example 2 were used in an antigen dependent suppression assay to determine whether HLA-A2 TFP Treg can suppress activation of effector T cells co-cultured with mismatched dendritic cells. CD3+ HLA-A2-CD4+ and CD8+ effector T cells generated from the same donor as the TFP Treg. HLA-A2+ dendritic cells were generated by isolating CD14+ monocytes and culturing in MO-DC media for 7 days. Effector T cells were cocultured with HLA-A2+ dendritic cells at a Teff:DC ratio of 2:1 (50,000 effector T cells and 25,000 dendritic cells per well). HLA-A2 TFP regulatory T cells with or without FoxP3, or CD4+ HLA-A2 TFP T cells with Fox P3 were then added to the coculture at various concentrations to measure the suppressive effect of the HLA-A2 TFP Treg or CD4+ cells on the effector T cells. T effector cells were also cultured alone or with HLA matched or mismatched dendritic cells without T regulatory cells as controls. Supernatants were taken for cytokine analysis and flow analysis was done on the responder cells to assess expansion after 72 hours of incubation. A schematic of the assay is shown in FIG. 14.

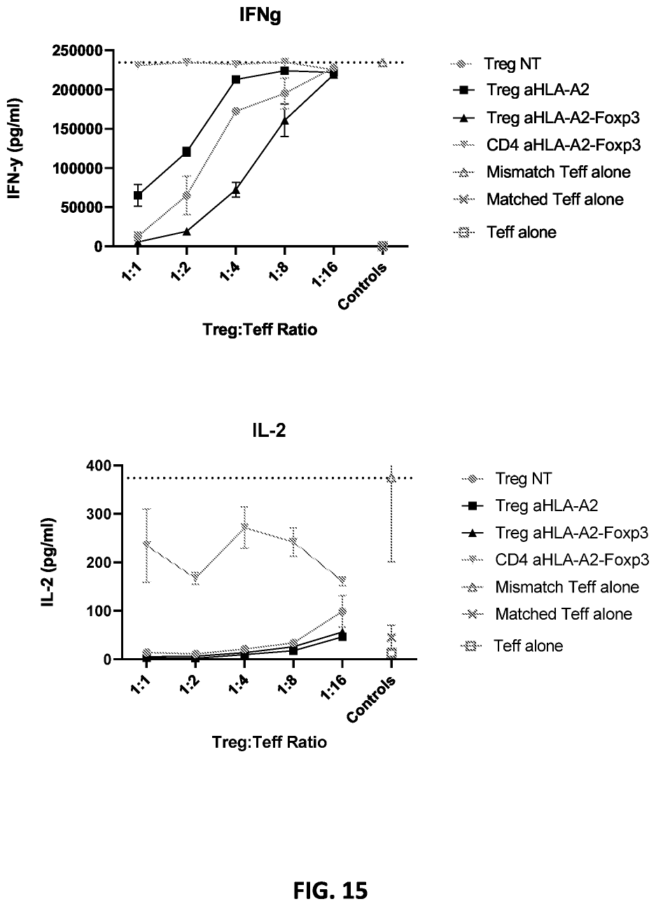

For cytokine analysis, levels of IFN-gamma and IL-2 were measured at each of the suppressor TFP T cell:effector T cell ratios. As is shown in FIG. 15, for both IFN-gamma and IL-2, HLA-A2 TFP Treg with or without the added FoxP3 gene were able to suppress IFN-gamma and IL-2 production of the responder cells (relative to the level of cytokines produced by effector T cells not treated with Treg or CD4+ T cells). Moreover, HLA-A2 TFP FoxP3 Treg had a greater suppressive effect than unmodified Treg on IFN-gamma production, and HLA-A2 TFP Treg with or without FoxP3 both had a greater suppressive effect on IL-2 production than unmodified T cells, indicating that TFP Treg can suppress cytokine production by effector T cells in an antigen-specific manner.

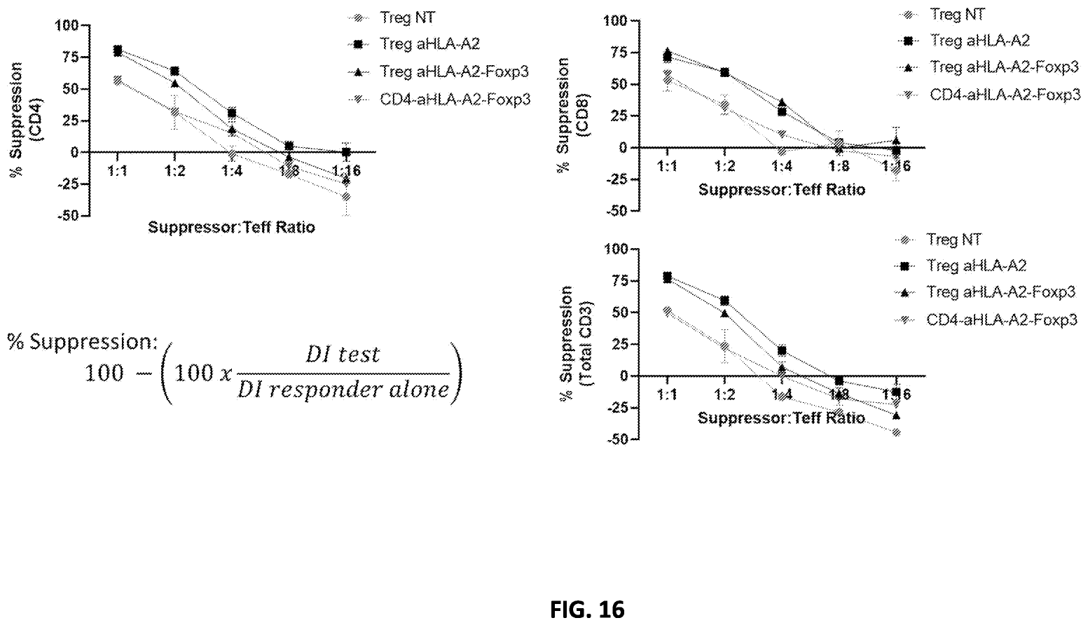

Expansion rate of the responder cells was also evaluated by FACS at each of the TFP T cell:effector cell ratios by quantifying the % suppression, as is calculated by the formula shown in FIG. 16. As is shown in FIG. 16, HLA-A2 TFP Treg with or without the added FoxP3 gene were able to suppress expansion of effector T cells and the level of suppression was above that achieved by unmodified regulatory T cells. This suggests that TFP Treg can suppress effector T cell expansion in an antigen dependent manner.

### Example 5: Modified Expansion Protocol for TFP Treg

A second expansion protocol for TFP Treg was developed to enhance expansion of TFP Treg.

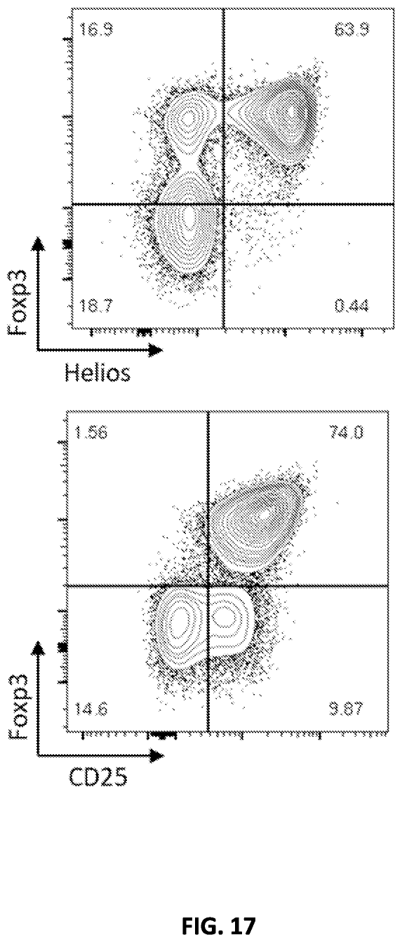

PBMCs were isolated using the MultiMACs and frozen. Cells were thawed overnight and CD4+ T cells were isolated from peripheral blood mononuclear cells (PBMCs) on an AutoMACs. Total CD4+ T cells were isolated with CD4 Release Beads (Miltenyi). A portion of these cells were taken and used as the CD4+ cells in the experiments below. CD4+ CD25+CD127dim/− Treg were then isolated using the by CD4+ CD25+ CD127dim/− Treg isolation kit II (Miltenyi). At days 0, prior to transduction, isolated Tregs were characterized by flow cytometry for expression of Helios, CD25, and FoxP3. As is shown in FIG. 17, isolated Tregs showed high levels of Helios and FoxP3 expression.

On day 0, isolated Treg were activated with Treg expansion beads (Miltenyi) in X-Vivo media+ 10% FBS+ 1000 IU/ml IL-2+ 100 nM Rapamycin. On day 1, activated T cells were transduced with lentivirus encoding the HLA-A2 TFP (as described in Example 2) with or without FoxP3. On days 4, 6, 8, and 11, the cells were washed, subcultured in fresh medium with cytokines and then expanded up to day 14. At each day of subculture, cells were harvested, washed, and resuspended with fresh cytokine-containing medium. CD4+ cells were activated, transduced and expanded as described in Example 2.

### Verification of TFP Expression and Phenotyping of TFP T Cells

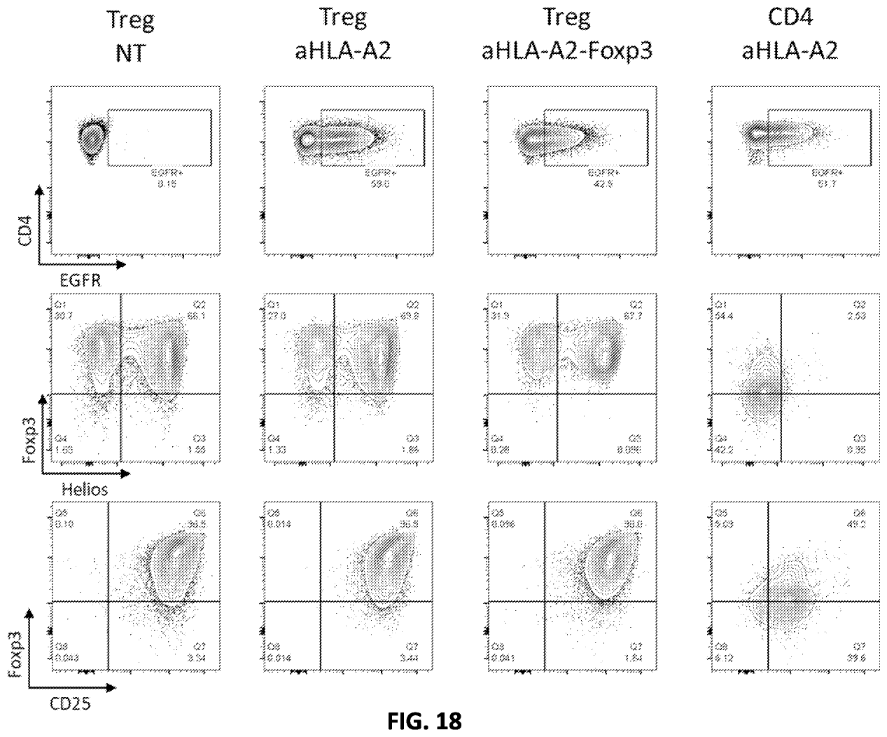

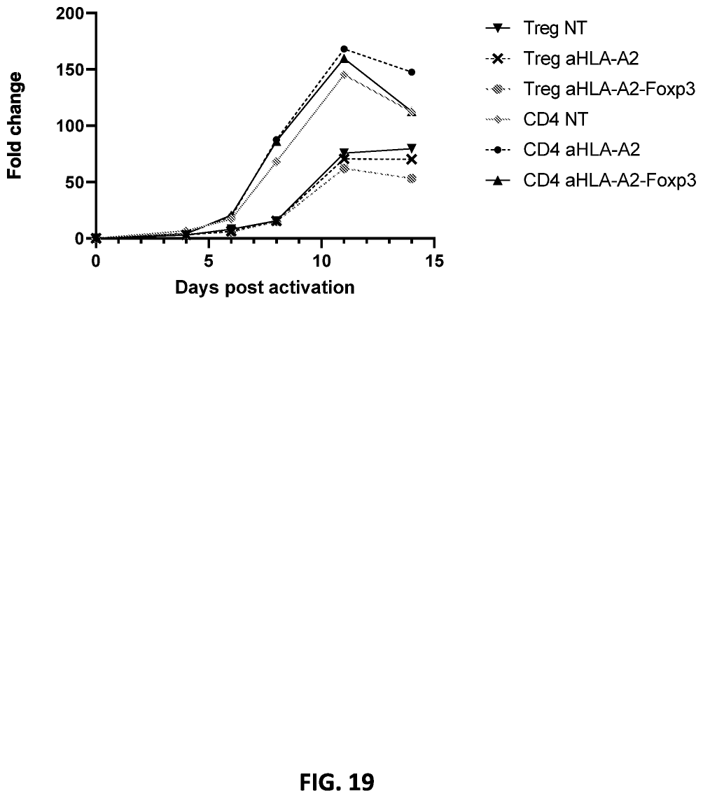

Phenotyping of the HLA-A2 TFP transduced T cells was performed. At day 14 of expansion T cells were harvested and the cells were characterized by flow cytometry for expression of FoxP3, CD25, and Helios. Verification of TFP expression was also confirmed in cells by anti-EGFR staining. As is shown in FIG. 18, nontransduced Treg and HLA-A2 TFP Treg, with and without the added FoxP3 gene have high levels of FoxP3, Helios, and CD25 expression. A high proportion of all TFP transduced cells show EGFR staining, suggesting high transduction efficiency. The expansion rates of transduced and non-transduced CD4 and Treg cells are shown in FIG. 19. For all Tregs, the expansion at day 10 is roughly 5× that seen with the protocol used in Example 2.

### Example 6: In Vivo Test for TFP.Anti-HLA-A2 Tree Function

The ability of TFP.anti-HLA-A2 Tregs to alter the onset or duration of rejection of graft cells by a host is determined. A humanized mouse model system is used to test the function of the TFP.anti-HLA-A2 Tregs. In a human xenograft transplant model, whether adoptive transfer of TFP Tregs alleviates Graft-versus-host-disease (GVHD that is caused by transferring allogeneic peripheral blood mononuclear cells (PBMCs) is determined. Mice that lack mature T, B, and NK cells, but may contain dysfunctional monocytic cells are irradiated 1 day prior to implantation of PBMCs. On Day 0, HLA-A2+ PBMCs and TFP anti-HLA-A2 Tregs with or without added Foxp3 and CD4+ TFP anti-HLA-A2 cells will be transplanted into irradiated mice at a 1:1 ratio of PBMC:TFP cells. Following the transplant, the mice are monitored daily for the development of GVHD. Blood is taken weekly to monitor PBMC and suppressor cell engraftment throughout the study. Mice are followed for up to 49 days or until they reach a humane endpoint.

### Other Embodiments

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. A pharmaceutical composition comprising
(I) a T regulatory cell (Treg) from a human subject, wherein the T regulatory cell comprises:
(a) a recombinant nucleic acid comprising a sequence encoding a T cell receptor (TCR) fusion protein (TFP) comprising:
(i) a TCR-integrating subunit comprising:
(1) an extracellular domain,
(2) a TCR transmembrane domain, and
(3) a TCR intracellular domain comprising a stimulatory domain from an intracellular signaling domain;
(ii) a binding domain; and
(II) a pharmaceutically acceptable carrier;
wherein the TCR-integrating subunit and the binding domain are operatively linked; and
wherein the TFP functionally interacts with an endogenous TCR when expressed in a T cell.

2. The pharmaceutical composition of claim 1, wherein the binding domain is selected from:
(a) an antigen binding domain:
(b) a T cell receptor ligand, e.g., a peptide-MHC complex; or
(c) a T cell receptor mimic, e.g., that binds the peptide-MHC complex.

3. The pharmaceutical composition of claim 1 or 2, wherein the Treg further comprises a gene that stimulates and/or stabilizes the formation of Tregs.

4. The pharmaceutical composition of claim 3, wherein the gene that stimulates and/or stabilizes the formation of Tregs is encoded by the same recombinant nucleic acid molecule as the recombinant nucleic acid molecule encoding the TFP.

5. The pharmaceutical composition of claim 3, wherein the gene that stimulates and/or stabilizes the formation of Tregs is encoded by a different recombinant nucleic acid molecule than the recombinant nucleic acid molecule encoding the TFP.

6. The pharmaceutical composition of any one of claims 3-5, wherein the gene that stimulates and/or stabilizes the formation of Tregs is FOXP3, HELIOS, BACH2, or pSTAT5.

7. The pharmaceutical composition of any one of claims 1-6, wherein the Treg further comprises a switch receptor.

8. The pharmaceutical composition of claim 7, wherein the switch receptor is encoded by the same recombinant nucleic acid molecule as the recombinant nucleic acid molecule encoding the TFP.

9. The pharmaceutical composition of claim 7, wherein the switch receptor is encoded by a different recombinant nucleic acid molecule than the recombinant nucleic acid molecule encoding the TFP.

10. The pharmaceutical composition of any one of claims 7-9, wherein the switch receptor is an IL7-IL2 switch receptor, an IL7-IL10 switch receptor, or a TNF-alpha-IL2 switch receptor.

11. The pharmaceutical composition of any one of claims 1-10, wherein the Treg comprises more than one gene that stimulates and/or stabilizes the formation of Tregs and/or more than one switch receptor.

12. The pharmaceutical composition of any one of claims 1-11, wherein the expression of one or more of PKC theta, STUB1, and CCAR2 in the Treg cell is reduced or eliminated.

13. The pharmaceutical composition of any one of claims 1-12, wherein the expression of one or more of CDK8 and CDK19 reduced, deleted, or pharmacologically inhibited to stabilized Treg formation.

14. The pharmaceutical composition of any one of claims 2-13, wherein the peptide of the peptide-MHC complex is an autoantigen or a fragment thereof.

15. The pharmaceutical composition of any one of claims 2-13, wherein the peptide of the peptide-MHC complex is an exogenous antigen or a fragment thereof.

16. The pharmaceutical composition of any one of claims 1-13, wherein the binding domain comprises an antigen binding domain.

17. The pharmaceutical composition of claim 16, wherein the antigen binding domain comprises an autoantigen binding domain or an exogenous antigen binding domain.

18. The pharmaceutical composition of claim 17, wherein the autoantigen binding domain specifically binds an autoantigen.

19. The pharmaceutical composition of claim 17, wherein the exogenous antigen binding domain specifically binds an exogenous antigen.

20. The pharmaceutical composition of any one of claims 14-19, wherein the autoantigen is one or more of islet glucose-6-phosphatase catalytic subunit related protein (IGRP), insulin, HLA-A2, myelin, or alpha-gliadin or a fragment thereof.

21. The pharmaceutical composition of any one of claims 14-19, wherein the exogenous antigen is FVIII or a therapeutic macromolecule, e.g., a therapeutic polypeptide, or a fragment thereof.

22. The pharmaceutical composition of claim 16, wherein the antigen binding domain binds to a cell membrane associated antigen.

23. The pharmaceutical composition of claim 16, wherein the antigen binding domain binds to a circulating antigen.

24. The pharmaceutical composition of any of claim 16, wherein the antigen binding domain is specific to an antigen on an islet cell.

25. The pharmaceutical composition of any one of claims 2-24, wherein the antigen binding domain is an antibody or functional fragment thereof.

26. The pharmaceutical composition of claim 25, wherein the antibody or functional fragment thereof is an scFv or a single domain antibody.

27. The pharmaceutical composition of claim 25 or 26, wherein the antibody or functional fragment thereof is human or humanized.

28. The pharmaceutical composition of any one of claims 1-15, 20, or 21, wherein the binding domain is a TCR mimic, e.g., specifically binds a peptide-MHC-complex.

29. The pharmaceutical composition of any one of claims 1-28, wherein the pharmaceutical composition reduces cytokine production of an effector T cell having the antigen, the MHC-peptide complex, or the T cell receptor that specifically binds the MHC-peptide complex, relative to a pharmaceutical composition having a Treg that does not contain the TFP.

30. The pharmaceutical composition of any one of claims 1-29, wherein the Treg is a CD4⁺ CD25⁺ FoxP3⁺ Treg or a CD8⁺ regulatory T cell.

31. The pharmaceutical composition of any one of claims 1-30, wherein the intracellular signaling domain is selected from CD3 gamma, CD3 delta, CD3 epsilon, and CD3 zeta.

32. The pharmaceutical composition of any one of claims 1-31, wherein the TCR-integrating subunit comprises (i) a TCR extracellular domain, (ii) a TCR transmembrane domain, and (iii) a TCR intracellular domain, wherein at least two or three of (i), (ii), and (iii) are from the same TCR subunit.

33. The pharmaceutical composition of any one of claims 1-32, wherein the binding domain is operatively linked to the TCR extracellular domain by a linker sequence.

34. The pharmaceutical composition of claim 32, wherein the linker sequence comprises (G₄S)ₙ, wherein n=1 to 4.

35. The pharmaceutical composition of any one of claims 1-34, wherein the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit and functional fragments thereof.

36. The pharmaceutical composition of any one of claims 1-35, wherein the TFP includes a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, a CD3 zeta TCR subunit and functional fragments thereof.

37. The pharmaceutical composition of any one of claims 1-36, wherein the TFP comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d and functional fragments thereof.

38. The pharmaceutical composition of claim 37, wherein the ITAM replaces an ITAM of CD3 gamma, CD3 delta, or CD3 epsilon.

39. A recombinant nucleic acid comprising a sequence encoding the TFP of the pharmaceutical composition of any one of claims 1-38.

40. The recombinant nucleic acid of claim 39, wherein the nucleic acid is selected from the group consisting of a DNA and an RNA.

41. The recombinant nucleic acid of claim 39 or 40, wherein the nucleic acid is an mRNA.

42. The recombinant nucleic acid of claim 39 or 40, wherein the nucleic acid is circRNA.

43. The recombinant nucleic acid of any one of claims 39-42, wherein the recombinant nucleic acid comprises a nucleic acid analog, wherein the nucleic acid analog is not in an encoding sequence of the recombinant nucleic acid.

44. The recombinant nucleic acid of any one of claims 39-43, further comprising a leader sequence.

45. The recombinant nucleic acid of any one of claims 39-44, further comprising a promoter sequence.

46. The recombinant nucleic acid of any one of claims 39-45, further comprising a sequence encoding a poly(A) tail.

47. The recombinant nucleic acid of any one of claims 39-46, further comprising a 3′UTR sequence.

48. The recombinant nucleic acid of any one of claims 39-47, wherein the nucleic acid is an isolated nucleic acid or a non-naturally occurring nucleic acid.

49. The recombinant nucleic acid molecule of any one of claims 39-48, wherein the nucleic acid is an in vitro transcribed nucleic acid.

50. A vector comprising the recombinant nucleic acid of any one of claims 1-49.

51. The vector of claim 50, wherein the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector.

52. The vector of claim 50 or 51, wherein the vector is an in vitro transcribed vector.

53. A circular RNA comprising the recombinant nucleic acid of any one of claims 39-49.

54. A method of treating or preventing a disease or disorder comprising administering to a subject in need thereof a therapeutically effective amount of the pharmaceutical composition of any one of claims 1-38.

55. The method of claim 54, wherein the disease or disorder is an autoimmune disease.

56. The method according to claim 55, wherein the autoimmune disease is an autoantibody-mediated autoimmune disease.

57. The method of claim 55, wherein the autoimmune disease is selected from the group comprising multiple sclerosis, autoimmune hemolytic anemia, celiac disease, and chronic inflammatory demyelinating polyradiculoneuropathy.

58. The method of claim 55, wherein the disease or disorder is inflammation, e.g., an inflammatory disease or disorder, an allergic reaction, or transplant rejection.

59. A composition for use in treating or preventing a disease or disorder in a subject in need thereof, comprising the recombinant nucleic acid of any one of claims 39-49 or the T cell of the pharmaceutical composition of any one of claims 1-38.

60. The composition of claim 59, wherein the disease or disorder is an autoimmune disease.

61. The composition of claim 59, wherein the disease or disorder is inflammation, e.g., an inflammatory disease or disorder, an allergic reaction, or transplant rejection.

62. The method or composition of any one of claims 54-61, wherein the subject has or is at risk of developing an autoimmune disease, inflammation, e.g., an inflammatory disease or disorder, an allergic reaction, or transplant rejection.

63. A T regulatory cell (Treg) from a human subject, wherein the T regulatory cell comprises a recombinant nucleic acid comprising a sequence encoding a T cell receptor (TCR) fusion protein (TFP) comprising:
(i) a TCR-integrating subunit comprising:
(1) at least a portion of a TCR extracellular domain, and
(2) a TCR transmembrane domain, and
(ii) a binding domain;
wherein the TCR-integrating subunit and the binding domain are operatively linked; and
wherein the TFP functionally interacts with an endogenous TCR when expressed in a T cell.

64. The T regulatory cell of claim 63, wherein the TFP further comprises a TCR intracellular signaling domain.

65. The T regulatory cell of claim 63 or 64, wherein the binding domain is selected from:
(a) an antigen binding domain:
(b) a T cell receptor ligand, e.g., a peptide-MHC complex; or
(c) a T cell receptor mimic, e.g., that binds the peptide-MHC complex.

66. The T regulatory cell of any one of claims 63-65, wherein the Treg further comprises a gene that stimulates and/or stabilizes the formation of Tregs.

67. The T regulatory cell of claim 66, wherein the gene that stimulates and/or stabilizes the formation of Tregs is encoded by the same recombinant nucleic acid molecule as the recombinant nucleic acid molecule encoding the TFP.

68. The T regulatory cell of claim 66, wherein the gene that stimulates and/or stabilizes the formation of Tregs is encoded by a different recombinant nucleic acid molecule than the recombinant nucleic acid molecule encoding the TFP.

69. The T regulatory cell of any one of claims 66-68, wherein the gene that stimulates and/or stabilizes the formation of Tregs is FOXP3, HELIOS, BACH2, or pSTAT5.

70. The T regulatory cell of any one of claims 63-69, wherein the Treg further comprises a switch receptor.

71. The T regulatory cell of claim 70, wherein the switch receptor is encoded by the same recombinant nucleic acid molecule as the recombinant nucleic acid molecule encoding the TFP.

72. The T regulatory cell of claim 70, wherein the switch receptor is encoded by a different recombinant nucleic acid molecule than the recombinant nucleic acid molecule encoding the TFP.

73. The T regulatory cell of any one of claims 70-72, wherein the switch receptor is an IL7-IL2 switch receptor, an IL7-IL10 switch receptor, or a TNF-alpha-IL2 switch receptor.

74. The T regulatory cell of any one of claims 63-73, wherein the Treg comprises more than one gene that stimulates and/or stabilizes the formation of Tregs and/or more than one switch receptor.

75. The T regulatory cell of any one of claims 63-74, wherein the expression of one or more of PKC theta, STUB1, and CCAR2 in the Treg cell is reduced or eliminated.

76. The T regulatory cell of any one of claims 63-75, wherein the expression of one or more of CDK8 and CDK19 reduced, deleted, or pharmacologically inhibited to stabilized Treg formation.

77. The T regulatory cell of any one of claims 65-76, wherein the peptide of the peptide-MHC complex is an autoantigen or a fragment thereof.

78. The T regulatory cell of any one of claims 65-76, wherein the peptide of the peptide-MHC complex is an exogenous antigen or a fragment thereof.

79. The T regulatory cell of any one of claims 63-78, wherein the binding domain comprises an antigen binding domain.

80. The T regulatory cell of claim 79, wherein the antigen binding domain comprises an autoantigen binding domain or an exogenous antigen binding domain.

81. The T regulatory cell of claim 80, wherein the autoantigen binding domain specifically binds an autoantigen.

82. The T regulatory cell of claim 80, wherein the exogenous antigen binding domain specifically binds an exogenous antigen.

83. The T regulatory cell of any one of claims 77-82, wherein the autoantigen is one or more of islet glucose-6-phosphatase catalytic subunit related protein (IGRP), insulin, HLA-A2, myelin, or alpha-gliadin or a fragment thereof.

84. The T regulatory cell of any one of claims 78-82, wherein the exogenous antigen is FVIII or a therapeutic macromolecule, e.g., a therapeutic polypeptide, or a fragment thereof.

85. The T regulatory cell of claim 79, wherein the antigen binding domain binds to a cell membrane associated antigen.

86. The T regulatory cell of claim 79, wherein the antigen binding domain binds to a circulating antigen.

87. The T regulatory cell of claim 79, wherein the antigen binding domain is specific to an antigen on an islet cell.

88. The T regulatory cell of any one of claims 79-87, wherein the antigen binding domain is an antibody or functional fragment thereof.

89. The T regulatory cell of claim 88, wherein the antibody or functional fragment thereof is an scFv or a single domain antibody.

90. The T regulatory cell of claim 88 or 89, wherein the antibody or functional fragment thereof is human or humanized.

91. The T regulatory cell of any one of claims 63-78, 83, or 84, wherein the binding domain is a TCR mimic, e.g., specifically binds a peptide-MHC-complex.

92. The T regulatory cell of any one of claims 63-91, wherein the T regulatory cell reduces cytokine production of an effector T cell having the antigen, the MHC-peptide complex, or the T cell receptor that specifically binds the MHC-peptide complex, relative to a T regulatory cell having a Treg that does not contain the TFP.

93. The T regulatory cell of any one of claims 63-92, wherein the Treg is a CD4⁺ CD25⁺ FoxP3⁺ Treg or a CD8⁺ regulatory T cell.

94. The T regulatory cell of any one of claims 63-93, wherein the intracellular signaling domain is selected from the group consisting of CD3 gamma, CD3 delta, CD3 epsilon, and CD3 zeta.

95. The T regulatory cell of any one of claims 63-94, wherein the TCR-integrating subunit comprises (i) a TCR extracellular domain, (ii) a TCR transmembrane domain, and (iii) a TCR intracellular domain, wherein at least two or three of (i), (ii), and (iii) are from the same TCR subunit.

96. The T regulatory cell of any one of claims 63-95, wherein the binding domain is operatively linked to the TCR extracellular domain by a linker sequence.

97. The T regulatory cell of claim 96, wherein the linker sequence comprises (G₄S)ₙ, wherein n=1 to 4.

98. The T regulatory cell of any one of claims 63-97, wherein the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit and functional fragments thereof.

99. The T regulatory cell of any one of claims 63-98, wherein the TFP includes a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, and functional fragments thereof.

100. The T regulatory cell of any one of claims 63-99, wherein the TFP includes a TCR intracellular domain of a protein selected from the group consisting of TCR alpha, TCR beta, TCR gamma, TCR delta. a CD3 epsilon TCR subunit, a CD3 gamma TCR subunit, and a CD3 delta TCR subunit.

101. The T regulatory cell of any one of claims 63-100, wherein the TFP comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d and functional fragments thereof.

102. The T regulatory cell of claim 101, wherein the ITAM replaces an ITAM of CD3 gamma, CD3 delta, or CD3 epsilon.

103. The T regulatory cell of any one of claims 63-102, wherein the Treg is autologous.

104. The T regulatory cell of any one of claims 63-102, wherein the Treg is allogeneic.

105. A pharmaceutical composition comprising the T regulatory cell of any one of claims 63-104, and a pharmaceutically acceptable carrier.

106. A recombinant nucleic acid comprising a sequence encoding the TFP of the T regulatory cell of any one of claims 63-104.

107. The recombinant nucleic acid of claim 106, wherein the nucleic acid is selected from the group consisting of a DNA and an RNA.

108. The recombinant nucleic acid of claim 106 or 107, wherein the nucleic acid is an mRNA.

109. The recombinant nucleic acid of claim 106 or 107, wherein the nucleic acid is circRNA.

110. The recombinant nucleic acid of any one of claims 106-109, wherein the recombinant nucleic acid comprises a nucleic acid analog, wherein the nucleic acid analog is not in an encoding sequence of the recombinant nucleic acid.

111. The recombinant nucleic acid of any one of claims 106-110, further comprising a leader sequence.

112. The recombinant nucleic acid of any one of claims 106-111, further comprising a promoter sequence.

113. The recombinant nucleic acid of any one of claims 106-112, further comprising a sequence encoding a poly(A) tail.

114. The recombinant nucleic acid of any one of claims 106-113, further comprising a 3′UTR sequence.

115. The recombinant nucleic acid of any one of claims 106-114, wherein the nucleic acid is an isolated nucleic acid or a non-naturally occurring nucleic acid.

116. The recombinant nucleic acid of any one of claims 106-115, wherein the nucleic acid is an in vitro transcribed nucleic acid.

117. A vector comprising the recombinant nucleic acid of any one of claims 106-116.

118. The vector of claim 117, wherein the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector.

119. The vector of claim 117 or 118, wherein the vector is an in vitro transcribed vector.

120. A circular RNA comprising the recombinant nucleic acid of any one of claims 106-116.

121. A method of treating or preventing a disease or disorder comprising administering to a subject in need thereof a therapeutically effective amount of the T regulatory cell of any one of claims 63-104.

122. The method of claim 121, wherein the disease or disorder is an autoimmune disease.

123. The method of claim 122, wherein the autoimmune disease is an autoantibody-mediated autoimmune disease.

124. The method of claim 122, wherein the autoimmune disease is selected from the group comprising multiple sclerosis, autoimmune hemolytic anemia, celiac disease, and chronic inflammatory demyelinating polyradiculoneuropathy.

125. The method of claim 122, wherein the disease or disorder is inflammation, e.g., an inflammatory disease or disorder, an allergic reaction, or transplant rejection.

126. A composition for use in treating or preventing a disease or disorder in a subject in need thereof, comprising the recombinant nucleic acid of any one of claims 106-116 or the T regulatory cell of any one of claims 63-104.

127. The composition of claim 126, wherein the disease or disorder is an autoimmune disease.

128. The composition of claim 126, wherein the disease or disorder is inflammation, e.g., an inflammatory disease or disorder, an allergic reaction, or transplant rejection.

129. The method or composition of any one of claims 121-128, wherein the subject has or is at risk of developing an autoimmune disease, inflammation, e.g., an inflammatory disease or disorder, an allergic reaction, or transplant rejection.


SEQUENCE LISTING (83 sequences)

SEQ ID NO: 1 - DNA, 45 bp, Artificial Sequence
  Description of Artificial Sequence Synthetic oligonucleotide
gggggtggaggctctggagggggcggtagtggtggcggaggaagc

SEQ ID NO: 2 - DNA, 370 bp, Artificial Sequence
  Description of Artificial Sequence Synthetic polynucleotide
acaggtgcagctggtgcagtctgggggaggcgtggtccagcctggggggtccctgagagtctcctgtgcagcgtctggggtcaccctcagtgattatggcatgcattgggtccgccaggctccaggcaaggggctggagtggatggcttttatacggaatgatggaagtgataaatattatgcagactccgtgaagggccgattcaccatctccagagacaactccaagaaaacagtgtctctgcaaatgagcagtctcagagctgaagacacggctgtgtattactgtgcgaaaaatggcgaatctgggcctttggactactggtacttcgatctctggggccgtggcaccctggtcaccgtgtcgagt

SEQ ID NO: 3 - DNA, 324 bp, Artificial Sequence
  Description of Artificial Sequence Synthetic polynucleotide
gatgttgtgatgactcagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaggattttgcaacttattactgccaacaatatagtagttttccgctcactttcggcggagggaccaaagtggatatcaaacgt

SEQ ID NO: 4 - protein, 17 aa, Artificial Sequence
  Description of Artificial Sequence Synthetic peptide
GGGGSGGGGSGGGGSLE

SEQ ID NO: 5 - protein, 20 aa, Artificial Sequence
  Description of Artificial Sequence Synthetic peptide
AAAGGGGSGGGGSGGGGSLE

SEQ ID NO: 6 - protein, 30 aa, Artificial Sequence
  Description of Artificial Sequence Synthetic polypeptide
AAAIEVMYPPPYLGGGGSGGGGSGGGGSLE

SEQ ID NO: 7 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 9 - protein, 22 aa, Homo sapiens
MQSGTHWRVLGLCLLSVGVWGQ

SEQ ID NO: 10 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 11 - protein, 26 aa, Homo sapiens
VMSVATIVIVDICITGGLLLLVYYWS

SEQ ID NO: 12 - protein, 55 aa, Homo sapiens
KNRKAKAKPVTRGAGAGGRQRGQNKERPPPVPNPDYEPIRKGQRDLYSGLNQRRI

SEQ ID NO: 13 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 14 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 15 - protein, 22 aa, Homo sapiens
MEQGKGLAVLILAIILLQGTLA

SEQ ID NO: 16 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 17 - protein, 21 aa, Homo sapiens
GFLFAEIVSIFVLAVGVYFIA

SEQ ID NO: 18 - protein, 45 aa, Homo sapiens
GQDGVRQSRASDKQTLLPNDQLYQPLKDREDDQYSHLQGNQLRRN

SEQ ID NO: 19 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 20 - protein, 151 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 21 - protein, 21 aa, Homo sapiens
MEHSTFLSGLVLATLLSQVSP

SEQ ID NO: 22 - protein, 84 aa, Homo sapiens [sequence omitted]

SEQ ID NO: 23 - protein, 21 aa, Homo sapiens
GIIVTDVIATLLLALGVFCFA

SEQ ID NO: 24 - protein, 45 aa, Homo sapiens
GHETGRLSGAADTQALLRNDQVYQPLRDRDDAQYSHLGGNWARNK

SEQ ID NO: 25 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 27 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 115 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 29 - protein, 23 aa, Homo sapiens
VIGFRILLLKVAGFNLLMTLRLW

SEQ ID NO: 30 - protein, 2 aa, Homo sapiens
SS

SEQ ID NO: 31 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 32 - protein, 137 aa, Mus musculus
  MOD_RES (1)..(1): Any amino acid
XIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 33 - protein, 22 aa, Mus musculus
MGLRILLLKVAGFNLLMTLRLW

SEQ ID NO: 34 - protein, 2 aa, Mus musculus
SS

SEQ ID NO: 35 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 36 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 37 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 38 - protein, 150 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYE

SEQ ID NO: 39 - protein, 21 aa, Homo sapiens
ILLGKATLYAVLVSALVLMAM

SEQ ID NO: 40 - protein, 6 aa, Homo sapiens
VKRKDF

SEQ ID NO: 41 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 42 - protein, 27 aa, Mus musculus
ILYEILLGKATLYAVLVSTLVVMAMVK

SEQ ID NO: 43 - protein, 5 aa, Mus musculus
KRKNS

SEQ ID NO: 44 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 45 - protein, 138 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSA

SEQ ID NO: 46 - protein, 23 aa, Homo sapiens
YYMYLLLLLKSVVYFAIITCCLL

SEQ ID NO: 47 - protein, 12 aa, Homo sapiens
RRTAFCCNGEKS

SEQ ID NO: 48 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 49 - protein, 129 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 50 - protein, 23 aa, Homo sapiens
LGLRMLFAKTVAVNFLLTAKLFF

SEQ ID NO: 51 - protein, 1 aa, Homo sapiens
L

SEQ ID NO: 52 - protein, 338 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 53 - protein, 790 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 54 - protein, 926 aa, Artificial Sequence
  Description of Artificial Sequence Synthetic polypeptide
MLLLVTSLLLCELPHPAFLLIPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIKRAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 55 - protein, 1305 aa, Artificial Sequence
  Description of Artificial Sequence Synthetic polypeptide
MLLLVTSLLLCELPHPAFLLIPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIKRAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 56 - protein, 20 aa, Artificial Sequence
  Description of Artificial Sequence Synthetic peptide
AAAGGGGSGGGGSGGGGSLE

SEQ ID NO: 57 - protein, 30 aa, Artificial Sequence; identical to SEQ ID NO: 6
  Description of Artificial Sequence Synthetic polypeptide

SEQ ID NO: 58 - DNA, 20 bp, Artificial Sequence
  Description of Artificial Sequence Synthetic oligonucleotide
ctcgaccagcttgacatcac

SEQ ID NO: 59 - DNA, 20 bp, Artificial Sequence
  Description of Artificial Sequence Synthetic oligonucleotide
acactggtgtgcctggccac

SEQ ID NO: 60 - protein, 10 aa, Artificial Sequence
  Description of Artificial Sequence Synthetic peptide
GGDWSANFMY

SEQ ID NO: 61 - protein, 19 aa, Artificial Sequence
  Description of Artificial Sequence Synthetic peptide
RISGRGVVDYVESVKGRFT

SEQ ID NO: 62 - protein, 3 aa, Artificial Sequence
  Description of Artificial Sequence Synthetic peptide
ASY

SEQ ID NO: 63 - protein, 10 aa, Artificial Sequence
  Description of Artificial Sequence Synthetic peptide
GSTSSINTMY

SEQ ID NO: 64 - protein, 19 aa, Artificial Sequence
  Description of Artificial Sequence Synthetic peptide
FISSGGSTNVRDSVKGRFT

SEQ ID NO: 65 - protein, 11 aa, Artificial Sequence
  Description of Artificial Sequence Synthetic peptide
YIPYGGTLHDF

SEQ ID NO: 66 - protein, 10 aa, Artificial Sequence
  Description of Artificial Sequence Synthetic peptide
GSTFSIRAMR

SEQ ID NO: 67 - protein, 18 aa, Artificial Sequence
  Description of Artificial Sequence Synthetic peptide
VIYGSSTYYADAVKGRFT

SEQ ID NO: 68 - protein, 9 aa, Artificial Sequence
  Description of Artificial Sequence Synthetic peptide
DTIGTARDY

SEQ ID NO: 69 - protein, 111 aa, Artificial Sequence
  Description of Artificial Sequence Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 70 - protein, 119 aa, Artificial Sequence
  Description of Artificial Sequence Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 71 - protein, 116 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 72 - protein, 20 aa, Artificial Sequence
  Description of Artificial Sequence Synthetic peptide
  SITE (1)..(20): This sequence may encompass 1-4 “Gly Gly Gly Gly Ser” repeating units
GGGGSGGGGSGGGGSGGGGS

SEQ ID NO: 73 - protein, 40 aa, Artificial Sequence
  Description of Artificial Sequence Synthetic polypeptide
  SITE (1)..(40): This sequence may encompass 1-10 “Gly Gly Gly Ser” repeating units
  See specification as filed for detailed description of substitutions and preferred embodiments
GGGSGGGSGGGSGGGSGGGSGGGSGGGSGGGSGGGSGGGS

SEQ ID NO: 74 - protein, 20 aa, Artificial Sequence
  Description of Artificial Sequence Synthetic peptide
GGGGSGGGGSGGGGSGGGGS

SEQ ID NO: 75 - protein, 15 aa, Artificial Sequence
  Description of Artificial Sequence Synthetic peptide
GGGGSGGGGSGGGGS

SEQ ID NO: 76 - protein, 4 aa, Artificial Sequence
  Description of Artificial Sequence Synthetic peptide
GGGS

SEQ ID NO: 77 - protein, 20 aa, Artificial Sequence
  Description of Artificial Sequence Synthetic peptide
  SITE (1)..(20): This sequence may encompass 2-4 “Gly Gly Gly Gly Ser” repeating units
GGGGSGGGGSGGGGSGGGGS

SEQ ID NO: 78 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 79 - RNA, 5000 nt, Artificial Sequence
  Description of Artificial Sequence Synthetic polynucleotide
  misc_feature (1)..(5000): This sequence may encompass 50-5000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 80 - protein, 10 aa, Artificial Sequence
  Description of Artificial Sequence Synthetic peptide
GGGGSGGGGS

SEQ ID NO: 81 - protein, 50 aa, Artificial Sequence
  Description of Artificial Sequence Synthetic polypeptide
  SITE (1)..(50): This sequence may encompass 1-10 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGS

SEQ ID NO: 82 - DNA, 30 bp, Artificial Sequence
  Description of Artificial Sequence Synthetic oligonucleotide
ggtggcggaggttctggaggtggaggttcc

SEQ ID NO: 83 - RNA, 2000 nt, Artificial Sequence
  Description of Artificial Sequence Synthetic polynucleotide
  misc_feature (1)..(2000): This sequence may encompass 50-2000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa